SEQUENCE LISTING (85 sequences)

SEQ ID NO: 1 - DNA, 22 bp, Artificial Sequence
  Synthetic oligonucleotide
gatgaaatcggctcccgcagac

SEQ ID NO: 2 - DNA, 22 bp, Artificial Sequence Sequence
  Synthetic oligonucleotide
atgaaatcgactcccgcagaca

SEQ ID NO: 3 - DNA, 24 bp, Artificial Sequence Sequence
  Synthetic oligonucleotide
tggacaggcgaggaatacaggtat

SEQ ID NO: 4 - DNA, 26 bp, Artificial Sequence
  Synthetic oligonucleotide
ctggacaggcaaggaatacaggtatt

SEQ ID NO: 5 - DNA, 22 bp, Artificial Sequence
  Synthetic oligonucleotide
ctcagcgagcctcaatgctccc

SEQ ID NO: 6 - DNA, 23 bp, Artificial Sequence
  Synthetic oligonucleotide
ctctcagcaagcctcaatgctcc

SEQ ID NO: 7 - DNA, 47 bp, Artificial Sequence
  Synthetic oligonucleotide
tcctggaaccaatcccgtgaaagaattatttttgtgtttctaaaact

SEQ ID NO: 8 - DNA, 58 bp, Artificial Sequence
  Synthetic oligonucleotide
aaagatcccggggacgatggggcaagtgatgcccatgtcggtgcatgccttcacaaag

SEQ ID NO: 9 - DNA, 46 bp, Artificial Sequence
  Synthetic oligonucleotide
ccacagaaaatgatgcccagtgcttaacaagaccatactacagtga

SEQ ID NO: 10 - DNA, 47 bp, Artificial Sequence; identical to SEQ ID NO: 7
  Synthetic oligonucleotide

SEQ ID NO: 11 - DNA, 58 bp, Artificial Sequence; identical to SEQ ID NO: 8
  Synthetic oligonucleotide

SEQ ID NO: 12 - DNA, 45 bp, Artificial Sequence
  Synthetic oligonucleotide
ccacagaaaatgatgcccagtgcttaacaagaccatactacagtg

SEQ ID NO: 13 - DNA, 20 bp, Artificial Sequence
  Synthetic oligonucleotide
ggacaggcgaggaatacagg

SEQ ID NO: 14 - DNA, 21 bp, Artificial Sequence
  Synthetic oligonucleotide
tggacaggcaaggaatacagg

SEQ ID NO: 15 - DNA, 24 bp, Artificial Sequence
  Synthetic oligonucleotide
tggacaggcgaggaatacaggtat

SEQ ID NO: 16 - DNA, 26 bp, Artificial Sequence
  Synthetic oligonucleotide
ctggacaggcaaggaatacaggtatt

SEQ ID NO: 17 - DNA, 21 bp, Artificial Sequence
  Synthetic oligonucleotide
ttctatttccgtactactgac

SEQ ID NO: 18 - DNA, 27 bp, Artificial Sequence
  Synthetic oligonucleotide
ttctatttccgtactactgacgtaact

SEQ ID NO: 19 - DNA, 21 bp, Artificial Sequence
  Synthetic oligonucleotide
ccattcttctcaaactatcgt

SEQ ID NO: 20 - DNA, 21 bp, Artificial Sequence
  Synthetic oligonucleotide
ccattcttcactaactatcgc

SEQ ID NO: 21 - DNA, 22 bp, Artificial Sequence
  Synthetic oligonucleotide
cacactccattcttctcaaact

SEQ ID NO: 22 - DNA, 22 bp, Artificial Sequence
  Synthetic oligonucleotide
cacactccattcttcactaact

SEQ ID NO: 23 - DNA, 19 bp, Artificial Sequence
  Synthetic oligonucleotide
atatgacttcccaggtgac

SEQ ID NO: 24 - DNA, 18 bp, Artificial Sequence
  Synthetic oligonucleotide
gtagatacttacattcca

SEQ ID NO: 25 - DNA, 18 bp, Artificial Sequence
  Synthetic oligonucleotide
gttgatgattacattcca

SEQ ID NO: 26 - DNA, 21 bp, Artificial Sequence
  Synthetic oligonucleotide
ccattcttcactaactaccgc

SEQ ID NO: 27 - DNA, 22 bp, Artificial Sequence
  Synthetic oligonucleotide
catacgccattcttcactaact

SEQ ID NO: 28 - DNA, 21 bp, Artificial Sequence
  Synthetic oligonucleotide
ccattcttctctaactatcgt

SEQ ID NO: 29 - DNA, 21 bp, Artificial Sequence
  Synthetic oligonucleotide
ccattcttcacaaactatcgt

SEQ ID NO: 30 - DNA, 21 bp, Artificial Sequence
  Synthetic oligonucleotide
ccattcttcagtaactatcgc

SEQ ID NO: 31 - DNA, 21 bp, Artificial Sequence
  Synthetic oligonucleotide
ccattcttcagtaactaccgc

SEQ ID NO: 32 - DNA, 21 bp, Artificial Sequence
  Synthetic oligonucleotide
ccattcttctcaaactaccgc

SEQ ID NO: 33 - DNA, 21 bp, Artificial Sequence
  Synthetic oligonucleotide
ccattcttctctaactaccgt

SEQ ID NO: 34 - DNA, 22 bp, Artificial Sequence
  Synthetic oligonucleotide
catacgccattcttcagtaact

SEQ ID NO: 35 - DNA, 22 bp, Artificial Sequence
  Synthetic oligonucleotide
cacactccattcttcagtaact

SEQ ID NO: 36 - DNA, 22 bp, Artificial Sequence
  Synthetic oligonucleotide
catactccattcttcactaact

SEQ ID NO: 37 - DNA, 22 bp, Artificial Sequence
  Synthetic oligonucleotide
catacaccattcttctcaaact

SEQ ID NO: 38 - DNA, 22 bp, Artificial Sequence
  Synthetic oligonucleotide
catactccattcttctctaact

SEQ ID NO: 39 - DNA, 22 bp, Artificial Sequence
  Synthetic oligonucleotide
cacactccattcttcacaaact

SEQ ID NO: 40 - DNA, 22 bp, Artificial Sequence
  Synthetic oligonucleotide
cacactccattcttctctaact

SEQ ID NO: 41 - DNA, 18 bp, Artificial Sequence
  Synthetic oligonucleotide
tcgatggtaaaggttggc

SEQ ID NO: 42 - DNA, 24 bp, Artificial Sequence
  Synthetic oligonucleotide
atggcatgagtaacgaagaatata

SEQ ID NO: 43 - DNA, 21 bp, Artificial Sequence
  Synthetic oligonucleotide
aaagaacctctgctcaacaag

SEQ ID NO: 44 - DNA, 22 bp, Artificial Sequence
  Synthetic oligonucleotide
gcacttgtaagcacaccttcat

SEQ ID NO: 45 - DNA, 21 bp, Artificial Sequence
  Synthetic oligonucleotide
ttccagattacaacttcacca

SEQ ID NO: 46 - DNA, 19 bp, Artificial Sequence
  Synthetic oligonucleotide
gttcctccatatctctgcg

SEQ ID NO: 47 - DNA, 18 bp, Artificial Sequence
  Synthetic oligonucleotide
atttcaccgctacacatg

SEQ ID NO: 48 - DNA, 21 bp, Artificial Sequence
  Synthetic oligonucleotide
gtcagtagtacggaaatagaa

SEQ ID NO: 49 - DNA, 27 bp, Artificial Sequence
  Synthetic oligonucleotide
agttacgtcagtagtacggaaatagaa

SEQ ID NO: 50 - DNA, 21 bp, Artificial Sequence
  Synthetic oligonucleotide
acgatagtttgagaagaatgg

SEQ ID NO: 51 - DNA, 21 bp, Artificial Sequence
  Synthetic oligonucleotide
gcgatagttagtgaagaatgg

SEQ ID NO: 52 - DNA, 22 bp, Artificial Sequence
  Synthetic oligonucleotide
agtttgagaagaatggagtgtg

SEQ ID NO: 53 - DNA, 22 bp, Artificial Sequence
  Synthetic oligonucleotide
agttagtgaagaatggagtgtg

SEQ ID NO: 54 - DNA, 19 bp, Artificial Sequence
  Synthetic oligonucleotide
gtcacctgggaagtcatat

SEQ ID NO: 55 - DNA, 18 bp, Artificial Sequence
  Synthetic oligonucleotide
tggaatgtaagtatctac

SEQ ID NO: 56 - DNA, 18 bp, Artificial Sequence
  Synthetic oligonucleotide
tggaatgtaatcatcaac

SEQ ID NO: 57 - DNA, 21 bp, Artificial Sequence
  Synthetic oligonucleotide
gcggtagttagtgaagaatgg

SEQ ID NO: 58 - DNA, 22 bp, Artificial Sequence
  Synthetic oligonucleotide
agttagtgaagaatggcgtatg

SEQ ID NO: 59 - DNA, 21 bp, Artificial Sequence
  Synthetic oligonucleotide
acgatagttagagaagaatgg

SEQ ID NO: 60 - DNA, 21 bp, Artificial Sequence
  Synthetic oligonucleotide
acgatagtttgtgaagaatgg

SEQ ID NO: 61 - DNA, 21 bp, Artificial Sequence
  Synthetic oligonucleotide
gcgatagttactgaagaatgg

SEQ ID NO: 62 - DNA, 21 bp, Artificial Sequence
  Synthetic oligonucleotide
gcggtagttactgaagaatgg

SEQ ID NO: 63 - DNA, 21 bp, Artificial Sequence
  Synthetic oligonucleotide
gcggtagtttgagaagaatgg

SEQ ID NO: 64 - DNA, 21 bp, Artificial Sequence
  Synthetic oligonucleotide
acggtagttagagaagaatgg

SEQ ID NO: 65 - DNA, 22 bp, Artificial Sequence
  Synthetic oligonucleotide
agttactgaagaatggcgtatg

SEQ ID NO: 66 - DNA, 22 bp, Artificial Sequence
  Synthetic oligonucleotide
agttactgaagaatggagtgtg

SEQ ID NO: 67 - DNA, 22 bp, Artificial Sequence
  Synthetic oligonucleotide
agttagtgaagaatggagtatg

SEQ ID NO: 68 - DNA, 22 bp, Artificial Sequence
  Synthetic oligonucleotide
agtttgagaagaatggtgtatg

SEQ ID NO: 69 - DNA, 22 bp, Artificial Sequence
  Synthetic oligonucleotide
agttagagaagaatggagtatg

SEQ ID NO: 70 - DNA, 22 bp, Artificial Sequence
  Synthetic oligonucleotide
agtttgtgaagaatggagtgtg

SEQ ID NO: 71 - DNA, 22 bp, Artificial Sequence
  Synthetic oligonucleotide
agttagagaagaatggagtgtg

SEQ ID NO: 72 - DNA, 18 bp, Artificial Sequence
  Synthetic oligonucleotide
gccaacctttaccatcga

SEQ ID NO: 73 - DNA, 24 bp, Artificial Sequence
  Synthetic oligonucleotide
tattgtattcgttactcatgccat

SEQ ID NO: 74 - DNA, 21 bp, Artificial Sequence
  Synthetic oligonucleotide
cttgttgagcagaggttcttt

SEQ ID NO: 75 - DNA, 22 bp, Artificial Sequence
  Synthetic oligonucleotide
atgaaggtgtgcttacaagtgc

SEQ ID NO: 76 - DNA, 21 bp, Artificial Sequence
  Synthetic oligonucleotide
tggtgaagttgtaatctggaa

SEQ ID NO: 77 - DNA, 19 bp, Artificial Sequence
  Synthetic oligonucleotide
cgcagagatatggaggaac

SEQ ID NO: 78 - DNA, 18 bp, Artificial Sequence
  Synthetic oligonucleotide
catgtgtagcggtgaaat

SEQ ID NO: 79 - DNA, 280 bp, Staphylococcus aureus
atccaccctcaaacaggtgaattattagcattgtaagcacaccttcatatgacgtctatccatttatgtatggcatgagtaacgaagaatataataaattaaccgaagataaaaaagaacctctgctcaagtcaatccagattacaacttcaccaggttcaactcaaaaaatattaacagcaatgattgggttaaataacaaaacattagacgataaaacaagttataaaatcgatggtaaaggttggcaaaaagataaatcttggggtggttacaacgt

SEQ ID NO: 80 - DNA, 478 bp, Staphylococcus aureus
cgagaccaagattcaacaagccaagtgaaacaaatgcatacaacgtaacgacaaatcaagatggcacagtatcatacggagctcgcccaacacaaaacaagccaagtgaaaaaacgcatataacgtaacaacacatgcaaatggtcaagtatcatacggtgctcgcccaacacaaacaagccaagcaaaacaaatgcatacaacgtaacaacacatgcaaatggtcaagtatcatatggcgctcgcccgacacaaaaaaagccaagcaaaacaaatgcatataacgtaacaacacatgcaaatggtcaagtatcatacggagctcgcccgacatacaagaagccaagcgaaacaaatgcatacaacgtaacaacacatgcaaatggtcaagtatcatatggcgctcgcccgacacaaaaaaagccaagcgaaacaaacgcatataacgtaacaacacatgcagatggtactgcgacat

SEQ ID NO: 81 - DNA, 142 bp, Staphylococcus aureus
gtggtcaagtattagctgctcctggttcaattacaccacatactgaattcaaagcagaagtatacgtattatcaaaagacgaaggtggacgtcacactccattcttctcaractatcgtccacaattctatttccgtactac

SEQ ID NO: 82 - DNA, 373 bp, Staphylococcus aureus
tgatgccrgttgaggacgtattctcaatcactggtcgtggtactgttgctacaggccgtgttgaacgtggtcaaatcaaagttggtgaagaagttgaaatcatcggtttacatgacacatctaaaacaactgttacaggtgttgaaatgttccgtaaattattagactacgctgaagctggtgacaacattggtgcattattacgtggtgttgctcgtgaagacgtacaacgtggtcaagtattagctgctcctggttcaattacaccacatactgaattcaaagcagaagtatacgtattatcaaaagacgaaggtggacgtcacactccattcttctcaractatcgtccacaattctatttccgtactac

SEQ ID NO: 83 - DNA, 373 bp, Staphylococcus epidermidis
tgatgccagttgaggacgtattctcaatcactggtcgtggtactgttgctacaggccgtgttgaacgtggtcaaatcaaagttggtgaagaagttgaaatcatcggtatgcacgaaacttctaaaacaactgttactggtgtagaaatgttccgtaaattattagactacgctgaagctggtgacaacatcggtgctttattacgtggtgttgcacgtgaagacgtacaacgtggtcaagtattagctgctcctggttctattacaccacacacaaaattcaaagctgaagtatacgtattatctaaagatgaaggtggacgtcacactccattcttcactaactatcgcccacaattctatttccgtactac

SEQ ID NO: 84 - DNA, 380 bp, Staphylococcus saprophyticus
tgatgccagttgaggacgtattctcaatcactggtcgtggtactgttgctacaggccgtgttgaacgtggtcaaatcaaagtcggtgaagaaatcgaaatcatcggtatgcaagaagaatcaagcaaaacaactgttactggtgtagaaatgttccgtaaattattagactacgctgaagctggtgacaacattggtgcattattacgtggtgtttcacgtgatgatgtacaacgtggtcaagttttagctgctcctggtactatcacaccacatacaaaattcaaagcggatgtttacgttttatctaaagatgaaggtggtcgtcatacgccattcttcactaactaccgcccacaattctatttccgtactactgac

SEQ ID NO: 85 - DNA, 451 bp, Staphylococcus aureus
cgccgcgtgagtgatgaaggtcttcggatcgtaaaactctgttattagggaagaacaaacgtgtaagtaactgtgcacgtcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgcc

(19) United States
(12) Patent Application Publication
(10) Pub. No.: US 2007/0154903 A1
(43) Pub. Date: Jul. 5, 2007

(54) Selective isolation and concentration of nucleic acids from complex samples

(75) Inventors: Sudhakar S. Marla, Northbrook, IL (US); Anna L. Prokhorova, Elmhurst, IL (US); Susan Hetzel, Eden Prairie, MN (US); William Cork, Lake Bluff, IL (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Nanosphere, Inc.

(21) Appl. No.: 11/473,996

(22) Filed: Jun. 23, 2006

Related U.S. Application Data
(60) Provisional application No. 60/693,491, filed on Jun. 23, 2005.

(51) Int. Cl. C12Q 1/68; C12M 3/00

(52) U.S. Cl. 435/6; 435/287.2; 977/924

(57) ABSTRACT

The present invention provides methods for detecting a target nucleic acid molecule in a sample that comprises nucleic acid molecules of higher biological complexity than that of amplified nucleic acid molecules in a complex environment containing numerous non-nucleic acid components. In particular, the present invention provides methods and probes for isolating DNA with detergents and detecting a single nucleotide polymorphism (SNP) in a complex sample that comprises numerous non-nucleic acid components and nucleic acid molecules of higher biological complexity than that of amplified nucleic acid molecules.

### CROSS-REFERENCE TO RELATED APPLICATIONS

This application claims the benefit if U.S. Provisional Application No. 60/693,491, filed Jun. 23, 2005, which is incorporated by reference in its entirety.

### FIELD OF THE INVENTION

The invention relates to a method for detection of a target nucleic acid molecule in a sample that comprises nucleic acid molecules of higher biological complexity than that of amplified nucleic acid molecules, for example in genomic DNA. In particular, the invention relates to methods and probes for SNP detection using nanoparticle-labeled probes. The invention also relates to methods for detecting biological organisms, and in particular, bacterial pathogens such as Staphylococcal DNA in a sample and for detecting antibiotic resistance genes such as the mecA gene, which confers resistance to the antibiotic methicillin. The invention further provides methods by which nucleic acids molecules can be selectively isolated and concentrated from a variety of complex samples by using a combination of lysis-fragmentation and condensing buffers comprising among other components cetyl trimethylammonium bromide (CTAB).

### BACKGROUND OF THE INVENTION

Single nucleotide polymorphisms (SNPs) or single base variations between genomic DNA observed in different individuals not only form the basis of genetic diversity, they are expected to be markers for disease propensity, to allow better disease management, to enhance understanding of disease states and to ultimately facilitate the discovery of more effective drugs. As a consequence, numerous efforts are ongoing with the common goal of developing methods that reliably and rapidly identify SNPs. The majority of these efforts require target amplification by methods such as PCR because of the inherent complexity of human genomic DNA (haploid genome=3×10⁹ bp) and the associated sensitivity requirements. The ability to detect SNPs directly in human genomic DNA would simplify the assay and eliminate target amplification-related errors in SNP identification.

Single nucleotide polymorphisms can be identified by a number of methods, including DNA sequencing, restriction enzyme analysis, or site-specific hybridization. However, high-throughput genome-wide screening for SNP and mutations requires the ability to simultaneously analyze multiple loci with high accuracy and sensitivity. To increase sensitivity as well as specificity, current high-throughput methods for single nucleotide detection rely on a step that involves amplification of the target nucleic acid sample, usually by the polymerase chain reaction (PCR) (see, e.g., Nikiforov et al., U.S. Pat. No. 5,679,524 issued Oct. 21, 1997; McIntosh et al., PCT publication WO 98/59066 dated Dec. 30, 1998; Goelet et al., PCT publication WO 95/12607 dated May 11, 1995; Wang et al., 1998, Science 280:1077-1082; Tyagi et al., 1998, Nature Biotechnol. 16:49-53; Chen et al., 1998, Genome Res. 8:549-556; Pastinen et al., 1996, Clin. Chem. 42:1391-1397; Chen et al, 1997, Proc. Natl. Acad. Sci. 94:10756-10761; Shuber et al., 1997, Hum. Mol. Gen. 6:337-347; Liu et al., 1997, Genome Res. 7:389-398; Livak et al., Nature Genet. 9:341-342; Day and Humphries, 1994, Anal. Biochem. 222:389-395). Typically, there are two reasons why PCR amplification is necessary for conventional hybridization based SNP detection. First, when obtaining total human DNA, which has a size of 3,000,000,000 base pairs per haploid genome, the target sequence containing the SNP site represents only a very small fraction of the total DNA. For instance, a 20 base target sequence represents only 0.00000033% of the total DNA (for a normal genome there are two copies of that target sequence, but they may have different SNP sites and are therefore considered different sites). Thus, a typical DNA sample of a few micrograms may be insufficient for many of the current techniques for lack of sensitivity. A more important reason, however, is that hybridization to that 20 base target sequence with oligonucleotides that are sufficiently short to allow single base discrimination do not hybridize exclusively to the target region, but bind to a small extent to other regions in the genome. Given the overwhelming amount of non-target DNA, the non-specific hybridization creates such a large background that it buries the specific signal. Thus, a PCR amplification of one target region is a necessary step to dramatically reduce the amount of non-specific sequences. This amplification step is referred to as “complexity reduction.” However, the fidelity of the PCR technique is limited. Combinations of pairs of PCR primers tend to generate spurious reaction products or fail in some particular regions. Moreover, the number of errors in the final reaction product increases exponentially with each round of PCR amplification after a non-target sequence has been copied, or if an error has been introduced into the target sequence because of mis-incorporation. Thus, PCR errors can be a substantial drawback when searching for rare variations in nucleic acid populations.

Finally, a drawback of using target amplification is that each SNP site has to be separately amplified. Since there are potentially millions of SNPs in the human genome, this becomes an insurmountable task. Even the “state of the art” amplification methods and strategies that partially circumvent the problem of SNP-site-specific amplification can identify only a small percentage of the total number of SNPs simultaneously (less than 0.1%) (see e.g. Kennedy et al., 2003, Nature Biotechnol. 21:1233-1237). Eric Lander of the Whitehead Institute for Biomedical Research and one of the leaders of the human genome project cited elimination of target amplification as one of the most significant challenges in genome wide screening of SNPs (see Lander E. 1999, Nature Genetics Suppl. 21: 3-4). Thus, there remains a need in the art for more sensitive, effective, and cost efficient methods for detecting SNPs in a sample that do not require target amplification or complexity reduction.

The identification of DNA mutations is also critical for identifying biological microorganisms (see Edwards et. al, J. Clin. Micro. 39: 3047-3051). For example, the genus Staphylococcus contains at least 38 different species, and a large number of these species have been identified in hospital-based infections (Edwards et. al, J. Clin. Micro. 39: 3047-3051). Therefore, the rapid identification and speciation of organisms is critical for identifying the source of infection which helps determine patient treatment, and epidemiologically for recognizing outbreaks of infection and cross transmission of nosocomial pathogens (Olive and Bean, 1999, J. Clin. Micro. 37: 1661-1669). Conventional methods for identifying bacteria based on biochemical tests are often lengthy (>1 day) and often do not enable accurate identification of specific species (Hamels et. al, 2001, Biotechniques 31: 1364-1372). Therefore, significant effort has been devoted to developing more rapid, accurate, and less expensive methods for identifying specific bacterial species based on identification of nucleic acid sequences, especially in the case of nosocomial pathogens such as Staphylococcus. Microorganisms of the same family or genus contain phylogenetically conserved genes that encode for the same protein (Hamels et. al, 2001, Biotechniques 31: 1364-1372). Although the gene sequences from the same family are typically highly conserved, species-specific sequence mutations within a variety of genes (e.g. 16S rRNA) have been identified. Oligonucleotide probes which target a variable region of the 16S rRNA gene have been developed to identify a variety of coagulase negative and positive Staphylococcus species in real time PCR assays (Edwards et. al, J. Clin. Micro. 39: 3047-3051).

Furthermore, microarrays have been developed to identify the genus Staphylococcus, species, and antibiotic resistance using PCR-amplified femA gene sequences (Hamels et. al, 2001, Biotechniques 31: 1364-1372). The microarrays contained oligonucleotide probes which recognized species specific sequence variations in the femA gene (sequence variation of three bases or greater) associated with the five most clinically relevant Staphylococcus species (S. aureus, S. epidermidis, S. haemolyticus, S. hominis, S. saprophyticus), while an oligonucleotide probe that targeted a conserved region of the same gene was used to identify the genus Staphylococcus. However, one of the major drawbacks of both the microarray and real time PCR based assays is the requirement of PCR which can be less than ideal both clinically and from a cost perspective (see above PCR discussion for SNP identification).

Another problem in detecting SNPs and detecting and classifying microorganisms is sample preparation. Target nucleic acids are present with other molecules and sample preparation requires specifically enriching the sample with the target and eliminating non-target molecules. Traditionally, sample preparation uses methods that involve cell lysis and protein digestion followed by organic extraction of the target material. However, DNA isolated by the classical method is tedious because it requires an organic extraction step followed by DNA fragmentation and precipitation before the DNA can be used. Further, the quality of the DNA is such that high test-well backgrounds, high non-specific binding, false positive and negative results are common.

Thus, there remains a need in the art for more sensitive, effective, and cost efficient methods for selectively isolating DNA from complex samples, and detecting and speciating biological organisms in a sample that do not require target amplification or complexity reduction.

### SUMMARY OF THE INVENTION

The invention provides methods for detecting a target nucleic acid sequence in a sample, wherein the sample comprises nucleic acid molecules of higher biological complexity than that of amplified nucleic acid molecules and the target nucleic acid sequence differs from a known nucleic acid sequence by at least a single nucleotide. A single nucleotide difference, for example, can be a single nucleotide polymorphism.

In one aspect, the methods for detecting a target nucleic acid sequence in a sample without prior target amplification or complexity reduction comprise the steps of: a) admixing a sample to a lysis buffer, wherein the lysis buffer comprises at least one detergent; b) fragmenting the nucleic acids molecules of step (a); c) condensing the fragmented nucleic acid molecules onto a binding substrate in the presence of CTAB and NaCl so as to bind the nucleic acid molecules onto a surface of the substrate; d) washing the binding substrate having the bound nucleic acid molecules; e) eluting the bound nucleic acid molecules from the binding substrate; f) providing an addressable substrate having a capture oligonucleotide bound thereto, wherein the capture oligonucleotide has a sequence that is complementary to at least part of a first portion of the target nucleic acid sequence; g) providing a detection probe comprising detector oligonucleotides, wherein the detector oligonucleotides have sequences that are complementary to at least part of a second portion of the target nucleic acid sequence of step (f); h) contacting the nucleic acid molecules of step (e) with the substrate and the detection probe under conditions that are effective for the hybridization of the capture oligonucleotide to the first portion of the target nucleic acid sequence and the hybridization of the detection probe to the second portion of the target nucleic acid sequence; and i) detecting whether the capture oligonucleotide and detection probe hybridized with the first and second portions of the target nucleic acid sequence.

In another aspect, the methods for detecting a target nucleic acid sequence in a sample without prior target amplification or complexity reduction comprise the steps of: a) admixing a sample to a lysis buffer, wherein the lysis buffer comprises at least one detergent; b) fragmenting the nucleic acids molecules of step (a); c) condensing the fragmented nucleic acid molecules onto a binding substrate in the presence of CTAB and NaCl so as to bind the nucleic acid molecules onto a surface of the substrate; d) washing the binding substrate having the bound nucleic acid molecules; e) eluting the bound nucleic acid molecules from the binding substrate; f) providing an addressable substrate having a plurality of capture oligonucleotides bound thereto, wherein the capture oligonucleotides have sequences that are complementary to one or more portions of the target nucleic acid sequence; g) providing a detector probe comprising detector oligonucleotides, wherein the detector oligonucleotides have sequences that are complementary to one or more portions of the target nucleic acid sequence of step (f) that are not recognized by a capture oligonucleotide on the substrate; h) contacting the nucleic acid molecules of step (e) with the substrate and the detector probe under conditions that are effective for the hybridization of the capture oligonucleotides to one or more portions of the target nucleic acid sequence and the hybridization of the detector probe to one or more portions of the target nucleic acid sequence that is not recognized by a capture oligonucleotide; and i) detecting whether the capture oligonucleotide and detector probe hybridized with the target nucleic acid sequence.

The invention also provides methods for identifying a single nucleotide polymorphism in a sample, wherein the sample comprises nucleic acid molecules of higher biological complexity relative to amplified nucleic acid molecules.

In one aspect, the methods for identifying a single nucleotide polymorphism in a sample without prior target amplification or complexity reduction comprise the steps of: a) admixing a sample to a lysis buffer, wherein the lysis buffer comprises at least one detergent; b) fragmenting the nucleic acids molecules of step (a); c) condensing the fragmented nucleic acid molecules onto a binding substrate in the presence of CTAB and NaCl so as to bind the nucleic acid molecules onto a surface of the substrate; d) washing the binding substrate having the bound nucleic acid molecules; e) eluting the bound nucleic acid molecules from the binding substrate; f) providing an addressable substrate having at least one capture oligonucleotide bound thereto, wherein the at least one capture oligonucleotide have sequences that are complementary to at least a part of a nucleic acid target that comprises a specific polymorphism; g) providing a detector probe having detector oligonucleotides bound thereto, wherein the detector oligonucleotides have sequences that are complementary to at least a portion of the nucleic acid target of step (f); h) contacting the nucleic acid molecules of step (e) with the substrate and the detector probe under conditions that are effective for the hybridization of the capture oligonucleotide to the nucleic acid target and the hybridization of the detector probe to the nucleic acid target; and i) detecting whether the capture oligonucleotide and detector probe hybridized with the nucleic acid target.

In another aspect, the methods for identifying a single nucleotide polymorphism in a sample without prior target amplification or complexity reduction comprise the steps of: a) admixing a sample to a lysis buffer, wherein the lysis buffer comprises at least one detergent; b) fragmenting the nucleic acids molecules of step (a); c) condensing the fragmented nucleic acid molecules onto a binding substrate in the presence of CTAB and NaCl so as to bind the nucleic acid molecules onto a surface of the substrate; d) washing the binding substrate having the bound nucleic acid molecules; e) eluting the bound nucleic acid molecules from the binding substrate; f) providing an addressable substrate having capture oligonucleotides bound thereto, wherein the capture oligonucleotides have sequences that are complementary to multiple portions of a nucleic acid target, each portion comprising a specific polymorphism; g) providing a detector probe comprising detector oligonucleotides, wherein the detector oligonucleotides have sequences that are complementary to at least a portion of the nucleic acid target of step (f) that is not recognized by a capture oligonucleotide on the substrate; h) contacting the nucleic acid molecules of step (e) with the substrate and the detector probe under conditions that are effective for the hybridization of the capture oligonucleotides to multiple portions of the nucleic acid target and the hybridization of the detector probe to the nucleic acid target; and i) detecting whether the capture oligonucleotide and detector probe hybridized with the nucleic acid target.

In one embodiment, the lysis buffer further comprises at least one protease and at least one salt. Preferably, the protease in the lysis buffer is selected from the group consisting of endoproteases (also referred to as proteinases) and exoproteases. More preferably, the exoproteases is selected from the group consisting of Proteinase K, Bromelain, papain, and ficin.

In another embodiment, the lysis buffer further comprises at least one salt. The salt in the lysis buffer serves to provide counterions and to regulate the ionic strength. Thus, the salt can be any ionizable compound, and includes without limitation, NaCl, KCl, or MgCl₂.

In another embodiment, the lysis buffer further comprises at least one salt and at least one polymeric compound. Polymeric compounds are defined as any polymeric alcohol that stabilizes the components in the lysis buffer. Preferably, the polymeric compound in the lysis buffer is selected from the group consisting of polyvinyl alcohol and polyethylene glycol.

In another embodiment, the lysis buffer further comprises at least one salt, at least one polymeric compound, at least one protease, and at least one lipase. Lipases hydrolyze lipid molecules in the sample to generate the corresponding alcohols and fatty acids thereby lowering the sample complexity.

In another embodiment, the lysis buffer further comprises at least one salt, at least one polymeric compound, at least one protease, and at least one mucolytic compound. Mucolytic compounds are defined as any agent that hydrolyzes polysaccharides that may be associated with buccal swab, mouthwash samples and saliva. Representative mucolytic compound may be selected from the group consisting of any small molecule such as N-Acetyl-L-cysteine and an enzyme such as lysozyme.

In another embodiment, the fragmentation is carried out in the presence of at least one oxidant, DNases, restriction enzymes, an acid or by ultrasonication. Preferably, the oxidant is selected from the group consisting of perborate, percarbonate, hydrogen peroxide, and peroxymonosulfate.

In another embodiment, the method further comprises adding an aqueous solution comprising CTAB and NaCl subsequent to step (b) but prior to step (c).

In another embodiment, step (a) admixing the sample to the lysis buffer and step (b) fragmenting the nucleic acid molecules are carried out in a single step, and wherein the lysis buffer further comprises at least one protease, at least one salt, and at least one oxidant.

In another embodiment, step (a) admixing the sample to the lysis buffer and step (b) fragmenting the nucleic acid molecules are carried out in a single step, wherein the lysis buffer further comprises at least one salt, and at least one oxidant.

In another embodiment, step (a) admixing the sample to the lysis buffer and step (b) fragmenting the nucleic acid molecules are carried out in a single step, and wherein the lysis buffer further comprises at least one protease, at least one salt, and at least one oxidant.

In another embodiment, step (a) admixing the sample to a lysis buffer, step (b) fragmenting and step (c) condensing the nucleic acid molecules are carried out in a single step, and wherein the lysis buffer further comprises at least one protease, at least one salt, at least one oxidant and CTAB.

In another embodiment, the binding substrate is magnetic microbeads containing a silica surface.

In another embodiment, washing of the binding substrate having the bound nucleic acid molecules comprises washing with 80% ethanol to remove excess CTAB.

In one embodiment, the nucleotide difference or Single Nucleotide Polymorphism of the target nucleic acid can be recognized by either the capture oligonucleotide bound to the substrate or by the detector oligonucleotides.

In another embodiment, the target nucleic acid molecules in a sample can comprise genomic DNA, genomic RNA, expressed RNA, plasmid DNA, mitochondrial or other cell organelle DNA, free cellular DNA, viral DNA or viral RNA, or a mixture of two or more of the above.

In one embodiment, a substrate used in a method of the invention can comprise a plurality of capture oligonucleotides, each of which can recognize one or more different single nucleotide polymorphisms or nucleotide differences, and the sample can comprise more than one nucleic acid target, each of which comprises a different single nucleotide polymorphism or nucleotide difference that can hybridize with one of the plurality of capture oligonculeotides. In addition, one or more types of detector probes can be provided in a method of the invention, each of which has detector oligonucleotides bound thereto that are capable of hybridizing with a different nucleic acid target.

In one embodiment, a sample can be contacted with the detector probe so that a nucleic acid target present in the sample hybridizes with the detector oligonucleotides on the detector probe, and the nucleic acid target bound to the detector probe can then be contacted with the substrate so that the nucleic acid target hybridizes with the capture oligonucleotide on the substrate. Alternatively, a sample can be contacted with the substrate so that a nucleic acid target present in the sample hybridizes with a capture oligonucleotide, and the nucleic acid target bound to the capture oligonucleotide can then be contacted with the detector probe so that the nucleic acid target hybridizes with the detector oligonucleotides on the detector probe. In another embodiment, a sample can be contacted simultaneously with the detector probe and the substrate.

In yet another embodiment, a detector oligonucleotide can comprise a detectable label. The label can be, for example, fluorescent, luminescent, phosphorescent, radioactive, or a nanoparticle, and the detector oligonucleotide can be linked to a dendrimer, a molecular aggregate, a quantum dot, or a bead. The label can allow for detection, for example, by photonic, electronic, acoustic, opto-acoustic, gravity, electro-chemical, electro-optic, mass-spectrometric, enzymatic, chemical, biochemical, or physical means.

In one embodiment, the detector probe can be a nanoparticle probe having detector oligonucleotides bound thereto. The nanoparticles can be made of, for example, a noble metal, such as gold or silver. A nanoparticle can be detected, for example, using an optical or flatbed scanner. The scanner can be linked to a computer loaded with software capable of calculating grayscale measurements, and the grayscale measurements are calculated to provide a quantitative measure of the amount of nucleic acid detected. Where the nanoparticle is made of gold, silver, or another metal that can promote autometallography, the substrate that is bound to the nanoparticle by means of a target nucleic acid molecule can be detected with higher sensitivity using silver stain. Alternatively, the substrate bound to a nanoparticle can be detected by detecting light scattered by the nanoparticle.

In another embodiment, oligonucleotides attached to a substrate can be located between two electrodes, the nanoparticles can be made of a material that is a conductor of electricity, and step (i) in the methods of the invention can comprise detecting a change in conductivity. In yet another embodiment, a plurality of oligonucleotides, each of which can recognize a different target nucleic acid sequence, are attached to a substrate in an array of spots and each spot of oligonucleotides is located between two electrodes, the nanoparticles are made of a material that is a conductor of electricity, and step (i) in the methods of the invention comprises detecting a change in conductivity. The electrodes can be made, for example, of gold and the nanoparticles are made of gold. Alternatively, a substrate can be contacted with silver stain to produce a change in conductivity.

In another embodiment, the methods of the invention can be used to distinguish between two or more species of a common genus. In one aspect, the species can differ by two or more non-consecutive nucleotides. In another aspect, the species can differ by two or more consecutive nucleotides.

In one embodiment, a target nucleic acid sequence of the invention can be a portion of a gene of a Staphylococcus bacterium. In one aspect of this embodiment, the Staphylococcus bacterium can be, for example, S. aureus, S. haemolyticus, S. epidermidis, S. lugdunensis, S. hominis, or S. saprophyticus. Thus, the methods of the invention can be used for Staphylococcus speciation (i.e. differentiating between different species of Staphylococcus bacteria).

In another embodiment, a target nucleic acid sequence of the invention can be a portion of the mecA gene. Thus, the methods of the invention can be used to identify methicillin resistant strains of bacteria.

In yet another embodiment of the invention, a target nucleic acid sequence, a capture oligonucleotide, and/or a detection oligonucleotide can comprise the sequence set forth in SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 24, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, or SEQ ID NO: 78.

Specific preferred embodiments of the present invention will become evident from the following more detailed description of certain preferred embodiments and the claims.

### BRIEF DESCRIPTION OF THE DRAWINGS

FIG. 1 shows a schematic representation of the single-step hybridization process of the invention.

FIG. 2 shows a schematic representation of the two-step hybridization process of the invention.

FIG. 3 illustrates schematically a hybridized complex of a nanoparticle-labeled detection probe, a wild-type or mutant capture probe bound to a substrate, and a wild-type target. For SNP detection, the assay is performed under appropriate experimental conditions that retain the perfectly matched complexes (left) while preventing the complex containing the mismatch from forming (right).

FIG. 4 illustrates SNP detection of the factor V gene (1691 G->A) with unamplified human genomic DNA [part (a)] or salmon sperm DNA [part (b)] on Superaldehyde® slides that have wild type or mutant factor V gene capture probes. Part (c) is a graph that summarizes a detection signal intensity analysis for human genomic DNA and nonspecific salmon sperm DNA in the presence of either the wild type or mutant capture probes.

FIG. 5 demonstrates the importance of adjusting the hybridization conditions in order to make the methods of the invention capable of discriminating between two target nucleic acids that differ by 1 nucleotide (the SNP site).

FIG. 6 shows that the array (capture probe sequences) and hybridization conditions can be designed such that more than one SNP type can be tested within the same array and under the same hybridization conditions, and that SNP discrimination is possible between wt and mutant DNA, independent of the input DNA.

FIG. 7 demonstrates SNP detection of the factor V mutant gene (1691 G->A) with unamplified human genomic DNA (part (a)) using hybridizations with various formamide concentrations on CodeLink® slides that have arrayed wild-type and mutant factor V gene capture probes. Part (b) is a graph that summarizes the detection signal intensity analysis for human genomic DNA following hybridizations with various formamide concentrations in the presence of either the wild-type or mutant capture probes.

FIG. 8 shows that under optimally tuned conditions, the human wt DNA generates a signal on the wt probes only, while the human mutant DNA generates a signal only at the mutant capture probes only.

FIG. 9 shows the quantitative data for the perfect (center) hybridization condition in FIG. 8.

FIG. 10 shows that SNP discrimination can be performed with very little (less than 1 microgram) total human DNA. It also demonstrates the importance of capture oligonucleotide design, and appropriate match of the stringency conditions to the length and nucleotide composition of the capture (and detection) probes.

FIG. 11 shows the results of SNP detection using methods of the invention in genomic DNA in 10 separate hybridizations on a single slide. After accounting for the standard deviation, the net signal intensities for the match and mismatch in 10 separate hybridization wells did not overlap, meaning that for each hybridization reaction the SNP genotype of the input DNA could be reliably determined. Because the intensities at the match and mismatch spots are significantly different for each hybridization reaction, the SNP genotype of the input DNA can be reliably determined. FIG. 12 shows the results of multiplex SNP identification in whole genomic DNA using methods of the invention, which detected genotypes of factor V, factor II, and MTHFR genes.

FIG. 13 shows the results of multiplex SNP detection in whole genomic DNA from patient sample GM16028, which demonstrates the ability of the methods of the invention to identify heterozygous SNP genotypes for factor V, factor II, and MTHFR genes in a single individual.

FIG. 14 shows the results of multiplex SNP detection in whole genomic DNA from patient sample GM00037, which demonstrates the ability of the methods of the invention to identify that a single individual is wild type for one gene (in this case factor V), heterozygous for another gene (in this case factor II), and mutant for a third gene (in this case MTHFR).

FIG. 15 shows results from three different investigators performing the methods of the invention on two separate patient samples.

FIG. 16 illustrates the specific detection of a mecA gene from Staphylococcus genomic DNA isolated from methicillin resistant (mecA +) S. aureus bacterial cells using mecA 2 and mecA 6 capture oligonucleotides immobilized on a glass slide, and gold nanoparticles labeled with mecA 4 as a detection probe. Staphylococcus genomic DNA isolated from methicillin sensitive (mecA −) S. aureus bacterial cells was used as a negative control. A known amount of PCR amplified mecA gene (281 base-pair fragment labeled MRSA 281 bp) was used as a positive control. Part (a) illustrates a series of scanned images from wells of a microarray containing differing amounts of methicillin resistant genomic DNA target (75-300 million copies), as well as positive and negative control samples. Part (b) is a graph representing data analysis of the samples. The net signal from methicillin resistant S. aureus genomic DNA is plotted by subtracting the signal from the corresponding negative control spots. In all plots, the horizontal black line represents three standard deviations over the negative control spots containing methicillin sensitive S. aureus genomic DNA. The Figure demonstrates specific detection of the mecA from total bacterial genomic DNA.

FIG. 17 illustrates staphylococcal speciation using PCR amplicons or genomic DNA from S. aureus and S. epidermidis (ATCC no. 700699 and 35984, respectively). For testing of total genomic DNA, a sonication step was performed to fragment the DNA sample prior to array hybridization. Part (a) is a series of scanned images from wells from a microarray containing either Tuf 372 bp amplicons or genomic DNA (300 ng, ˜8.0 E7 copies). Water (no target) was used as a control. The array plate included Tuf 3 and Tuf 4 capture probes bound thereto. Gold nanoparticle-labeled Tuf 2 probes were used as detection probes. Part (b) provides a graph representing data analysis of the samples shown in Part (a). The horizontal black line represents three standard deviations over the background. Part (c) Tuf 372 bp amplicons or genomic DNA (8.0 E7 copies). The array plated included Tuf 5 and Tuf 6 capture probes bound thereto. Part (d) provides a graph representing data analysis of the samples shown in Part (c). The horizontal black line represents three standard deviations over background.

FIG. 18 provides the sequences of S. aureus mecA 281 base pair, S. aureus coa 450 base pairs, S. aureus Tuf 142 base pairs, S. aureus Tuf 372 base pair and S. epidermidis Tuf 372 base pair PCR amplicons used in examples 4-6.

FIG. 19 illustrates staphylococcus speciation and mecA gene detection using PCR amplified targets taken from commercially available staphylococcus strains ATCC 35556, ATCC 35984, ATCC 12228, ATCC 700699, and ATCC 15305. Part (a) is a series of scanned images from wells from a microarray containing either the PCR products of the 16S, Tuf, or mecA genes representing the five genomic samples. Part (b) is a series of graphs representing data analysis of the five samples. In all plots, a horizontal black line represents three standard deviations over the background.

FIG. 20 illustrates staphylococcus speciation and mecA detection using sonicated genomic DNA targets taken from commercially available Staphylococcus strains ATCC 35984, ATCC 700699, and ATCC 12228. Part (a) is a series of scanned images from wells from a microarray containing genomic DNA from ATCC 35984, ATCC 700699, or ATCC 12228. The array plate included 16S 12, mecA 6, Tuf 3, Tuf 4, Tuf 10 capture probes with a negative hybridization control bound thereto. Gold nanoparticle-labeled 16S 13, mecA 4, and Tuf 2 probes were used as detection probes. Part (b) is a series of graphs representing data analysis of the three samples. In all plots, a horizontal black line represents three standard deviations over the background.

FIG. 21 is a graph that illustrates the sensitivity limit for mecA gene detection using a genomic DNA target. Data analysis of mecA gene detection in a genomic sample of ATCC 700699 using the sequences from Table 3 in 5×SCC, 0.05% Tween 20, 0.01% BSA, 15% v/v formamide and 200 pM nanoparticle probe at 45C for 1.5 hours. The graph shows a limit of detection at 330 fM in a 50 μl reaction (34 ng total genomic DNA). Three standard deviations over the background is represented by the horizontal at 80 in the plot.

FIG. 22 shows a schematic and results of PCR-less SNP discrimination with DNA isolated from buccal swabs. Part (a) is a Schematic of the overall assay process. The buccal sample can also be any sample that comprises complex DNA, for example, spent media, and blood. Parts (b-d) are images from the microarray chip assay showing PCR-less SNP discrimination with the input DNA from buccal swab samples. Part (e) is a description of the sub-array format. The test array contains 6 replicate capture spots each for the Major (wild-type) and Minor (mutant) genotype for two SNPs in the genes Factor V and Factor II, respectively, along with positive and negative control spots. Genotyping is assessed based on the quantitation of the signals from the spots after a gold nanoparticle probe-based PCR-less chip assay. Signals at capture spots associated only with the Major genotype indicate a homozygous wild-type genotype. Signals at both the Major and Minor capture strands indicate a heterozygous genotype. Signals at only the Minor capture strand indicate a homozygous mutant genotype. The samples comprise the following: part (b) comprises a control genomic DNA sample with homozygous wild-type genotype for both SNPs; part (c) comprises buccal swab samples with heterozygote genotype for Factor V and homozygous wild-type for Factor II; and part (d) comprises buccal swab samples with homozygous wild-type for both SNPs.

FIG. 23 shows the results of a PCR-less SNP assay showing SNP discrimination from a mouthwash sample. Note that as with the buccal swab sample, no fragmentation of the isolated genomic DNA was required prior to the chip assay. Part (a) comprises 5 ug of purified sonicated DNA as a control. Part (b) comprises DNA from 1 ml of mouthwash after isolation and concentration using CTAB. Part (c) is a description of the sub-array format for this experiment.

FIG. 24 shows the results of an agarose gel that illustrates that the genomic DNA obtained from buccal swabs and mouthwash samples isolated according to Examples 8 and 10 is enriched in shorter fragments. Part (a) shows the gel image of mouthwash DNA. Part (b) is the lane descriptions of mouthwash samples in part (a). The DNA was treated with and without acid for fragmenting after isolation and compared to DNA purchased from Sigma (Item D3160). Part (c) is a gel image of DNA from buccal swabs and mouthwash samples comprising unfragmented swab sample and mouthwash after isolation with and without sonication. Samples are compared to DNA purchased from Sigma (Item D3160). Part (d) is the lane descriptions of part (c).

FIG. 25 is a bar graph of the results of the detection of genomic DNA isolated from B. thuringiensis by using the magnetic bead-based isolation. The bacterial sample contained a very small amount of DNA (1×10⁵-5×10⁵ copies) in Tryptic Soy Broth culture media. The DNA was released by using a lysis buffer and isolated by the CTAB-containing condensing buffer. The isolated DNA was subjected to the PCR-less chip assay designed to detect organism-specific genes. The quantitated signals from the different conditions are compared to a control DNA consisting of the bacterial DNA isolated from water and from TSB using a standard extraction and isopropanol precipitation. The advantage of using condensing agents such as CTAB for isolating DNA from spent media is observed by comparing the almost complete absence of DNA when isopropanol precipitaion is used.

FIG. 26 shows the results of a microarray assay that illustrate that unfragmented DNA results in unpredictable non-specific binding which can result in mis-calls. For example, sample A possesses the major genotype for both SNPs but the results indicate heterozygote for SNP 1 and mutant for SNP 2-both calls are wrong. Fragmentation of DNA in Sample B gives results that indicate the correct genotypes, that is, major for SNP 1 and major for SNP 2.

FIG. 27 shows the results from PCR-less assays conducted using DNA from three buccal swab samples. Sample Swab A is DNA that was not fragmented and isolated. No SNP could be detected because of the high background. Sample Swab B is DNA from cells that were lysed with the fragmentation buffer, extracted with phenol-chloroform, and precipitated before assaying. Good SNP discrimination is observed. Sample Swab C is DNA from the same preparation as sample Swab B. The results show variability and the high backgrounds even after the phenol-chloroform extraction makes this protocol unreliable.

FIG. 28 shows a schematic of the CTAB/Magnetic bead-based isolation that eliminates an organic extraction step. The lysis-fragmentation buffer also accomplished DNA fragmentation.

FIG. 29 shows the results using a lysis-fragmentation buffer that causes cells to lyse, to release DNA, and to affect DNA fragmentation. The lysis-fragmentation buffer may comprise a combination of enzymes, detergents, and oxidants.

FIG. 30 is assay results that show that clear SNP discrimination is observable in SNP samples. Swab samples are wild-type for FV and FII genes (Hypercoagulation) and wild-type for E60X (Cystic Fibrosis).

FIG. 31 shows the high sensitivity, multiplex detection of bacterial agents in a complex sample. In the experiment, culture media containing extremely low copy number (20 attomolar) of the target bacterial agents was subjected to the lysis-fragmentation steps to cause cell lysis, DNA release, DNA fragmentation, and DNA isolation. Even at this low target concentration, the isolation procedure works extremely efficiently as the isolated target is easily detected in a chip assay. The samples contained bacterial agents including B. anthracis, B. thuringiensis, and F. tularensis at extremely low concentrations (10,000 copies in 800 μL or a concentration of 20 attomolar or 20×10⁻¹⁸ M) in culture media that contained various interferents. The samples were subjected to the lysis-binding isolation protocol. The DNA bound to the magnetic beads were released into a 50 μL assay volume indicating a near 16-fold target enrichment or concentration. The isolated DNA was tested in a PCR-less chip assay designed to detect multiple bacterial and viral agents. (a) Images showing multiplex detection of three bacterial agents. When all three agents are present, spots specific to all three agents light up. In the presence of individual agents only the spots specific to the individual agent lights up. (b) Quantitation associated with the above images showing good specific signals for the individual agents at 10,000 copies.

### DETAILED DESCRIPTION OF THE PREFERRED EMBODIMENTS

Unless otherwise required by context, singular terms shall include pluralities and plural terms shall include the singular.

As utilized in accordance with the present disclosure, the following terms, unless otherwise indicated, shall be understood to have the following meanings:

As used herein, a “nucleic acid sequence,” a “nucleic acid molecule,” or “nucleic acids” refers to one or more oligonucleotides or polynucleotides as defined herein. As used herein, a “target nucleic acid molecule” or “target nucleic acid sequence” refers to an oligonucleotide or polynucleotide comprising a sequence that a user of a method of the invention desires to detect in a sample.

The term “polynucleotide” as referred to herein means single-stranded or double-stranded nucleic acid polymers of at least 10 bases in length. In certain embodiments, the nucleotides comprising the polynucleotide can be ribonucleotides or deoxyribonucleotides or a modified form of either type of nucleotide. Said modifications include base modifications such as bromouridine, ribose modifications such as arabinoside and 2′,3′-dideoxyribose and internucleotide linkage modifications such as phosphorothioate, phosphorodithioate, phosphoroselenoate, phosphorodiselenoate, phosphoroanilothioate, phoshoraniladate and phosphoroamidate. The term “polynucleotide” specifically includes single and double stranded forms of DNA.

The term “oligonucleotide” referred to herein includes naturally occurring, and modified nucleotides linked together by naturally occurring, and/or non-naturally occurring oligonucleotide linkages. Oligonucleotides are a polynucleotide subset comprising members that are generally single-stranded and have a length of 200 bases or fewer. In certain embodiments, oligonucleotides are 10 to 60 bases in length. In certain embodiments, oligonucleotides are 12, 13, 14, 15, 16, 17, 18, 19, or 20 to 40 bases in length. Oligonucleotides may be single stranded or double stranded, e.g. for use in the construction of a gene mutant. Oligonucleotides of the invention may be sense or antisense oligonucleotides with reference to a protein-coding sequence.

The term “naturally occurring nucleotides” includes deoxyribonucleotides and ribonucleotides. The term “modified nucleotides” includes nucleotides with modified or substituted sugar groups and the like. The term “oligonucleotide linkages” includes oligonucleotide linkages such as phosphorothioate, phosphorodithioate, phosphoroselenoate, phosphorodiselenoate, phosphoroanilothioate, phoshoraniladate, phosphoroamidate, and the like. See, e.g., LaPlanche et al., 1986, Nucl. Acids Res., 14:9081; Stec et al., 1984, J. Am. Chem. Soc., 106:6077; Stein et al., 1988, Nucl. Acids Res., 16:3209; Zon et al., 1991, Anti-Cancer Drug Design, 6:539; Zon et al., 1991, OLIGONUCLEOTIDES AND ANALOGUES: A PRACTICAL APPROACH, pp. 87-108 (F. Eckstein, Ed.), Oxford University Press, Oxford England; Stec et al., U.S. Pat. No. 5,151,510; Uhlmann and Peyman, 1990, Chemical Reviews, 90:543, the disclosures of which are hereby incorporated by reference for any purpose. An oligonucleotide can include a detectable label to enable detection of the oligonucleotide or hybridization thereof.

An “addressable substrate” used in a method of the invention can be any surface capable of having oligonucleotides bound thereto. Such surfaces include, but are not limited to, glass, metal, plastic, or materials coated with a functional group designed for binding of oligonucleotides. The coating may be thicker than a monomolecular layer; in fact, the coating could involve porous materials of sufficient thickness to generate a porous 3-dimensional structure into which the oligonucleotides can diffuse and bind to the internal surfaces.

A “binding substrate” as used herein refers to any type of solid that provides a surface onto which nucleic acid molecules can bind to. An example of a binding substrate is magnetic beads, typically coated with a silica layer, which can provide a surface to bind condensed nucleic acid molecules. Magnetic beads offer a convenient way of isolating nucleic acids by using magnets that eliminate centrifugation steps. A binding substrate also includes other kinds of surfaces that can bind condensed nucleic acid molecules. For example, but not limited to, silica beads, silica beads functionalized with negatively charged polymers such as polyacrylic acid, silica slide substrates, for example, glass slides, and silica slide substrates coated with negatively charged polymers, for example, polyacrylic acid, etc., can be used as binding substrates. The binding substrate in free flowing form can be used in a column format and samples containing condensed nucleic acid molecules can be poured over the column. A polymeric material comprises any polymeric substance suitable for making a column. Examples of polymeric materials include without limitation, polyethylene glycol, polyethylene, polypropylene, agarose, sepharose, or polyacrylamide. Such polymeric materials are available as cross-linked entities to provide discrete beads, which may be used in the column format. Such beads are amenable for further modification with negatively charged polymers to increase their nucleic acid-binding efficiency.

The term “capture oligonucleotide” as used herein refers to an oligonucleotide that is bound to a substrate and comprises a nucleic acid sequence that can locate (i.e. hybridize in a sample) a complementary nucleotide sequence or gene on a target nucleic acid molecule, thereby causing the target nucleic acid molecule to be attached to the substrate via the capture oligonucleotide upon hybridization. Suitable, but non-limiting examples of a capture oligonucleotide include DNA, RNA, PNA, LNA, or a combination thereof. The capture oligonucleotide may include natural sequences or synthetic sequences, with or without modified nucleotides.

A “detection probe” of the invention can be any carrier to which one or more detection oligonucleotides can be attached, wherein the one or more detection oligonucleotides comprise nucleotide sequences complementary to a particular nucleic acid sequence. The carrier itself may serve as a label, or may contain or be modified with a detectable label, or the detection oligonucleotides may carry such labels. Carriers that are suitable for the methods of the invention include, but are not limited to, nanoparticles, quantum dots, dendrimers, semi-conductors, beads, up- or down-converting phosphors, large proteins, lipids, carbohydrates, or any suitable inorganic or organic molecule of sufficient size, or a combination thereof.

As used herein, a “detector oligonucleotide” or “detection oligonucleotide” is an oligonucleotide as defined herein that comprises a nucleic acid sequence that can be used to locate (i.e. hybridize in a sample) a complementary nucleotide sequence or gene on a target nucleic acid molecule. Suitable, but non-limiting examples of a detection oligonucleotide include DNA, RNA, PNA, LNA, or a combination thereof. The detection oligonucleotide may include natural sequences or synthetic sequences, with or without modified nucleotides.

As used herein, the terms “label” refers to a detectable marker that may be detected by photonic, electronic, opto-electronic, magnetic, gravity, acoustic, enzymatic, or other physical or chemical means. The term “labeled” refers to incorporation of such a detectable marker, e.g., by incorporation of a radiolabeled nucleotide or attachment to an oligonucleotide of a detectable marker.

A “sample” as used herein refers to any quantity of a substance that comprises nucleic acids and that can be used in a method of the invention. The term “sample” also refers to a sample that may be a complex sample that comprises nucleic acid molecules and non-nucleic acid molecules wherein the nucleic acid molecules may be of higher biological complexity relative to amplified nucleic acid molecules. For example, the sample can be a biological sample or can be extracted from a biological sample derived from humans, animals, plants, fungi, yeast, bacteria, viruses, tissue cultures or viral cultures, or a combination of the above. They may contain or be extracted from solid tissues (e.g. bone marrow, lymph nodes, brain, skin), body fluids (e.g. serum, blood, urine, siliva, sputum, seminal or lymph fluids), skeletal tissues, or individual cells. Alternatively, the sample can comprise purified or partially purified nucleic acid molecules and, for example, buffers and/or reagents that are used to generate appropriate conditions for successfully performing a method of the invention.

In one embodiment of the invention, the target nucleic acid molecules in a sample can comprise genomic DNA, genomic RNA, expressed RNA, plasmid DNA, cellular nucleic acids or nucleic acids derived from cellular organelles (e.g. mitochondria) or parasites, or a combination thereof.

As used herein, a “complex sample” refers to a sample with concentrations of non-nucleic acid material. For example, a complex sample may comprise concentrations polysaccharides, lipids, proteins, and other biological and non-biological materials. Complex samples can be derived from a number of sources, including but not limited to, living and nonliving matter, viruses, bacteria, plants and animals.

As used herein, the “biological complexity” of a nucleic acid molecule refers to the number of non-repeat nucleotide sequences present in the nucleic acid molecule, as described, for example, in Lewin, GENE EXPRESSION 2, Second Edition: Eukaryotic Chromosomes, 1980, John Wiley & Sons, New York, which is hereby incorporated by reference. For example, a simple oligonucleotide of 30 bases that contains a non-repeat sequence has a complexity of 30. The E. coli genome, which contains 4,200,000 base pairs, has a complexity of 4,200,000, because it has essentially no repeat sequences. The human genome, however, has on the order of 3,000,000,000 base pairs, much of which is repeat sequences (e.g. about 2,000,000,000 base pairs). The overall complexity (i.e. number of non-repeat nucleotides) of the human genome is on the order of 1,000,000,000.

The complexity of a nucleic acid molecule, such as a DNA molecule, does not depend on a number of different repeat sequences (i.e. copies of each different sequence present in the nucleic acid molecule). For example, if a DNA has 1 sequence that is a nucleotides long, 5 copies of a sequence that is b nucleotides long, and 50 copies of a sequence that is c nucleotides long, the complexity will be a+b+c, while the repetition frequencies of sequence a will be 1, b will be 5, and c will be 10.

The total length of different sequences within a given DNA can be determined experimentally by calculating the C₀t_{1/2} for the DNA, which is represented by the following formula,

$${Cot}_{1 / 2} \frac{1}{k}$$

where C is the concentration of DNA that is single stranded at time t_{1/2} (when the reaction is ½ complete) and k is the rate constant. A C₀t_{1/2} represents the value required for half reassociation of two complementary strands of a DNA. Reassociation of DNA is typically represented in the form of Cot curves that plot the fraction of DNA remaining single stranded (C/C₀) or the reassociated fraction (1-C/C₀) against the log of the Cot. Cot curves were introduced by Britten and Kohne in 1968 (1968, Science 161:529-540). Cot curves demonstrate that the concentration of each reassociating sequence determines the rate of renaturation for a given DNA. The C₀t_{1/2}, in contrast, represents the total length of different sequences present in a reaction.

The C₀t_{1/2} of a DNA is proportional to its complexity. Thus, determining the complexity of a DNA can be accomplished by comparing its C₀t_{1/2} with the C₀t_{1/2} of a standard DNA of known complexity. Usually, the standard DNA used to determine biological complexity of a DNA is an E. coli DNA, which has a complexity identical to the length of its genome (4.2×10⁶ base pairs) since every sequence in the E. coli genome is assumed to be unique. Therefore, the following formula can be used to determine biological complexity for a DNA.

$$\frac{{Cot}_{1 / 2} ( any DNA )}{{Cot}_{1 / 2} ( E . coli DNA )} = \frac{complexity ( any DNA )}{4.2 \times {10}^{6}}$$

In certain embodiments, the invention provides methods for reliable detection and discrimination (i.e. identification) of a target nucleic molecule having a nucleotide mutation (for example, a single nucleotide polymorphism) in total human DNA without the need for enzymatic complexity reduction by PCR or any other method that preferentially amplifies a specific DNA sequence. Specifically, the methods of the invention comprise a combination of hybridization conditions (including reaction volume, salts, formamide, temperature, and assay format), capture oligonucleotide sequences bound to a substrate, a detection probe, and a sufficiently sensitive means for detecting a target nucleic acid molecule that has been recognized by both the capture oligonucleotide and the detection probe.

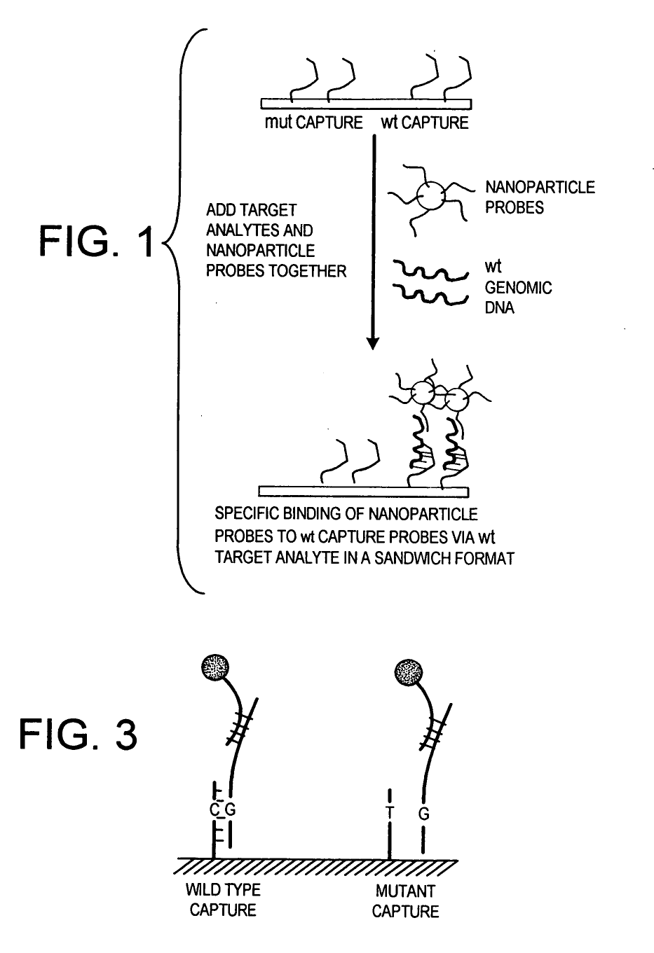

As demonstrated in the Examples, the invention provides for the first time a successful method for detecting a single nucleotide polymorphism in total human DNA without prior amplification or complexity reduction to selectively enrich for the target sequence, and without the aid of any enzymatic reaction, by single-step hybridization, which encompasses two hybridization events: hybridization of a first portion of the target sequence to the capture probe, and hybridization of a second portion of said target sequence to the detection probe. FIG. 1 shows a schematic representation of the single-step hybridization. As discussed above, both hybridization events happen in the same reaction. The target can bind to a capture oligonucleotide first and then hybridize also to a detection probe, such as the nanoparticle shown in the schematic, or the target can bind the detection probe first and then the capture oligonucleotide.

In another embodiment, the invention provides methods for reliable detection and discrimination (i.e. identification) of a target nucleic acid molecule having one or more non-consecutive nucleotide mutations in total DNA without the need for enzymatic complexity reduction by PCR or any other method that preferentially amplifies a specific DNA sequence. For example, the methods of the invention can be used to distinguish between two or more target nucleic acid molecules from two or more different species of a common genus, wherein the species differ by two or more non-consecutive nucleotides using capture oligonucleotides that differ by one or more nucleotides and/or detection oligonucleotides that differ by one or more nucleotides. The methods of the invention can also be used to distinguish between two or more species of a common genus that differ by two or more consecutive nucleotides.

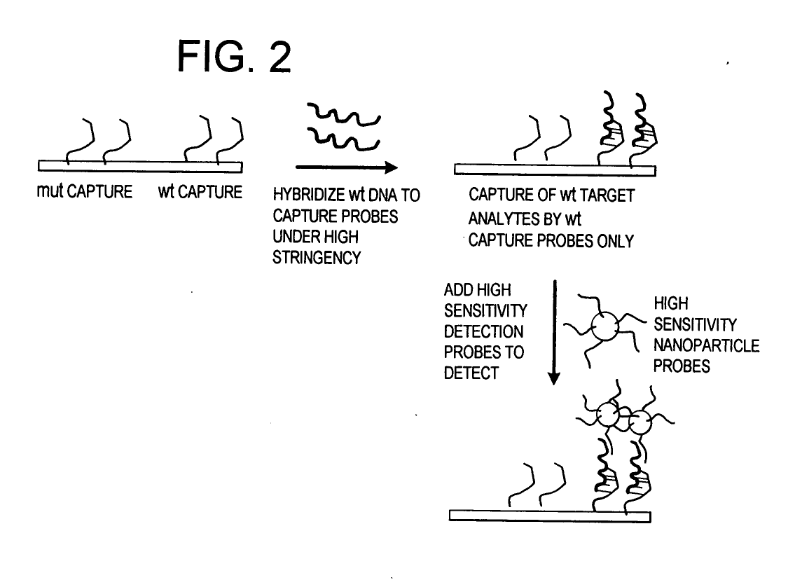

In one embodiment, the methods of the invention can be accomplished using a two-step hybridization. FIG. 2 shows a schematic representation of the two-step hybridization. In this process, the hybridization events happen in two separate reactions. The target binds to the capture oligonucleotides first, and after removal of all non-bound nucleic acids, a second hybridization is performed that provides detection probes that can specifically bind to a second portion of the captured target nucleic acid.

Methods of the invention that involve the two-step hybridization will work without accommodating certain unique properties of the detection probes (such as high Tm and sharp melting behavior of nanoparticle probes) during the first hybridization event (i.e. capture of the target nucleic acid molecule) since the reaction occurs in two steps. The first step is not sufficiently stringent to capture only the desired target sequences. Thus, the second step (binding of detection probes) is then provided to achieve the desired specificity for the target nucleic acid molecule. The combination of these two discriminating hybridization events allows the overall specificity for the target nucleic acid molecule. However, in order to achieve this exquisite specificity the hybridization conditions are chosen to be very stringent. Under such stringent conditions, only a small amount of target and detection probe gets captured by the capture probes. This amount of target is typically so small that it escapes detection by standard fluorescent methods because it is buried in the background. It is therefore critical for this invention to detect this small amount of target using an appropriately designed detection probe. The detection probes described in this invention consist in a carrier portion that is typically modified to contain many detection oligonucleotides, which enhances the hybridization kinetics of this detection probe. Second, the detection probe is also labeled with one or more high sensitivity label moieties, which together with the appropriate detection instrument, allows for the detection of the small number of captured target-detection probe complexes. Thus, it is the appropriate tuning of all factors in combination with a high sensitivity detection system that allows this process to work.

The two-step hybridization methods of the invention can comprise using any detection probes as described herein for the detection step. In a preferred embodiment, nanoparticle probes are used in the second step of the method. Where nanoparticles are used and the stringency conditions in the second hybridization step are equal to those in the first step, the detection oligonucleotides on the nanoparticle probes can be longer than the capture oligonucleotides. Thus, conditions necessary for the unique features of the nanoparticle probes (high Tm and sharp melting behavior) are not needed.

The single- and two-step hybridization methods in combination with the appropriately designed capture oligos and detection probes of the invention provide new and unexpected advantages over previous methods of detecting target nucleic acid sequences in a sample. Specifically, the methods of the invention do not require an amplification step to maximize the number of targets and simultaneously reduce the relative concentration of non-target sequences in a sample to enhance the possibility of binding to the target, as required, for example, in polymerase chain reaction (PCR) based detection methods. Specific detection without prior target sequence amplification provides tremendous advantages. For example, amplification often leads to contamination of research or diagnostic labs, resulting in false positive test outcomes. PCR or other target amplifications require specifically trained personnel, costly enzymes and specialized equipment. Most importantly, the efficiency of amplification can vary with each target sequence and primer pair, leading to errors or failures in determining the target sequences and/or the relative amount of the target sequences present in a genome. In addition, the methods of the invention involve fewer steps and are thus easier and more efficient to perform than gel-based methods of detecting nucleic acid targets, such as Southern and Northern blot assays.

In some embodiments, the addressable substrate having capture oligonucleotides bound thereto, comprises capture oligonucleotides that are complementary to one portion of the nucleic acid target or to multiple portions of a nucleic acid target, each portion comprising a specific polymorphism. The addressable substrate may comprise capture oligonucleotides that may be the same so that a single type of target is detected, or capture oligonucleotides that may be different so that multiple types of targets may be detected.

Isolating Nucleic Acid using CTAB

In one embodiment, the invention provides methods for detecting a target nucleic acid sequence in a sample comprising a step in which the nucleic acids can be selectively isolated and concentrated from a variety of complex samples by using a unique formulation of a lysis-fragmentation buffer in conjunction with a condensing buffer containing CTAB and magnetic beads that eliminates the need for organic extraction of the nucleic acid and centrifugation steps. Said advantages allow the isolation process to be easily automated. The isolated nucleic acids may be used in any detection assay platform, for example, in platforms for ultra-high sensitivity and specificity using gold nanoparticle probe technology. The isolated nucleic acids may also be used for detecting specific gene sequences or for identifying single base mis-matches in, for example, a microarray format. For instance, single base mis-matches from human genomic DNA can be identified. The isolated nucleic acid may also be useful to detect SNPs in human genomic DNA without the need for amplification or complexity reduction. DNA obtained from as little as 200-400 μL of blood can be used in multiplex PCR-less SNP detection systems.

In one embodiment, the invention provides for the isolation and concentration of genomic DNA (or RNA) from complex samples, for example, a biological sample. For example, genomic DNA (or RNA) may be isolated from a buccal swab or from a mouth wash sample. The isolation and concentration of the DNA comprises the use of a unique formulation of a lysis buffer in conjunction with one or more DNA condensing agent such as CTAB, salts, and proteases that together affect cell lysis and DNA release. The DNA is selectively condensed on to magnetic beads by using CTAB that retaining the interferents in solution. Successive washing under conditions wherein the DNA remains bound to the beads eliminates the interferents after which the pure DNA is eluted into a small elution volume. The isolated genomic DNA can be used without further manipulation in PCR applications as well as in high sensitivity PCR-less assays designed to identify SNPs, chromosomal abnormalities, or other nucleic acid characteristics. Additionally, the method allows successful isolation of DNA from very small amounts of bacterial cultures and spent media that can be detected directly in PCR-less assays.

Isolation of DNA for PCR-Less SNP Detection

DNA can be isolated from a variety of samples. In one embodiment, DNA is isolated from buccal swabs or mouthwash samples. Buccal swabs or mouthwash samples offer significant advantages over standard blood samples. For example, buccal swabs or mouthwash samples retrieval eliminate the need for specialized equipment and trained personnel. Moreover, buccal swabs or mouthwash samples are significantly safer as the risk of infection and accidental exposure to blood or to syringe needles is minimized.

DNA isolated from buccal swabs or mouthwash samples by the classical methods of cell lysis and protein digestion followed by organic extraction can be used as target material in the PCR-less assays. However, DNA isolated by the classical method is tedious because it requires an organic extraction step followed by DNA fragmentation and precipitation before the DNA can be used. Further, the quality of the DNA is such that high test-well backgrounds, high non-specific binding, false positive and negative results are common.

One way to eliminate the problems described above is to use the membrane solubilization properties and DNA condensation and precipitation properties of detergents under low-salt conditions. In one embodiment, detergents such as quaternary ammonium compounds may be used in a lysis buffer to solubilize membranes in samples and to condense and precipitation DNA. Preferably, the quaternary ammonium compound is CTAB. In conjunction with magnetic beads, the use of detergents eliminates the need for organic extraction and centrifugation steps typically used to separate DNA from polysaccharides, proteins, and lipid impurities.

In a one embodiment, a buccal swab sample is collected by rolling a sterile CytoSoft cytology brush (or equivalent) on the inside of each cheek 10-20 times. The collected sample is released into about 500 μL cell lysis buffer by using a twirling motion. Typically, the lysis buffer comprises proteases (for example, Protease mix (Qiagen) at a final concentration of 2-10 mg/mL or Proteinase K at 1-5 mg/mL). The sample is incubated for 10 minutes at 65° C. at which point a fragmentation buffer is added to the solution and the sample is incubated for an additional 5 minutes at 65° C. The fragmentation buffer comprises mild oxidants such as perborate or percarbonate and is formulated such that when added to the sample the concentration of the oxidants is about 0.1%-5% w/v, preferably about 0.5% w/v. In order to stabilize the oxidant, the pH is maintained about 8-9 by using borate buffers in the presence of polymers such as polyvinylalcohol (0.01% w/v). The fragmentation buffer effects DNA fragmentation (an important requirement for the PCR-less SNP detection assay). Next, the DNA is selectively isolated by using CTAB in conjunction with magnetic microbeads, typically containing a silica surface. Magnetic beads (20-100 μg) are added to the solution and the CTAB and NaCl concentrations were raised to about 1% and 0.3 M, respectively. The DNA is allowed to condense over the course of a short (about 10 minutes) incubation at 40° C. This is followed by an isolation of the magnetic beads with a magnetic separator. While the condensation does not require any alcohol, the DNA condensed on to the beads remained bound when washed with 80% ethanol. Repeated washing with 80% ethanol removes excess CTAB (or any other condensing agents) and the DNA is released into either water or a hybridization buffer (50 μL). The isolated DNA is ready to be tested in PCR-less SNP assays by using published protocols (see Bao et al. in Nucleic Acids Res. (2005) 33(2):e15, which is incorporated by reference in its entirety).

A lysis buffer comprises at least one detergent, at least one protease, and at least one salt. For example, the lysis buffer may comprise proteases (for example, Protease mix from Qiagen) at a final concentration of 2-10 mg/mL or Proteinase K at 1-5 mg/mL. The detergent may be any detergent, such as but not limited to, anionic, cationic or non-ionic detergents. For example, the detergent may be any of the commercially available detergents including, but not limited to, SDS, Tween 20, Tween 80, the MEGA series of detergents such as N-Decanoyl-N-methylglucamine, Igepal also known as NP 40 (DuPont), or a quaternary ammonium compound, such as CTAB. As used herein, lysis buffer refers to a formulation that accomplishes at least the task of releasing DNA from cells in a sample. Depending on the lysis buffer formulation, it may in addition accomplish DNA fragmentation, and referred to as lysis-fragmentation buffer. Or the lysis buffer may also accomplish DNA fragmentation and DNA isolation (condensation), and referred to as lysis-fragmentation-condensing buffer. In some instances, lysis and lysis-fragmentation buffers are used interchangeably, depending on the function performed by the buffers. The term fragmentation buffer refers to a formulation that accomplishes DNA fragmentation.

The lysis and fragmentation buffers can also comprise commercially available preparations such as stain removers. For example, samples collected by buccal swab can be released into a 2% v/v suspension of Shout® Liquid Laundry Stain Remover (S.C. Johnson & Son, Inc.). The sample is then heated at 65° C. for 10 minutes followed by addition of an aqueous solution of OxiClean® Original Formula (Orange Glo International) to a final concentration of 0.4% w/v. The solution is further incubated at 65° C. for 5 minutes. After incubation, the solution can be subjected to the CTAB-based magnetic isolation as described above. Several variations of this protocol along with different combinations of stain removers can also lead to successful DNA fragmentation and isolation.

The lysis and fragmentation buffers can also comprise other commercially available preparations that contain enzymes, detergents or oxidants such as percarbonates. For example, the lysis buffer may comprise Dreft and the fragmentation buffer may comprise Oxyboost. For a list of other commercially available preparations see, for example, the list found at the http protocol at the URL laundry-alternative.com/Oxygen_bleach_research.html.

The lysis buffer and fragmentation buffer can be formulated as one entity. In such instances, the buffer is referred to as the ‘lysis-fragmentation’ buffer. The formulation of the lysis-fragmentation buffer requires stabilizing the mild oxidants in the presence of detergents (and proteases, if needed since lysis of the cells is possible simply by the action of detergents).

The amount of detergents and proteases in the lysis buffer can be varied depending on the sample type (blood, saliva, mouthwash, etc) and the sample amount in order to provide optimal lysis and fragmentation. Thus, the same basic components of the lysis, fragmentation and lysis-fragmentation buffers may be used for obtaining fragmented DNA from varied sources. Further, instead of the mild oxidants in the fragmentation buffer, DNA fragmentation may be affected by the use of DNases or other restriction enzymes.

A condensing agent is any compound that causes DNA to condense and become insoluble in solution. The condensing agent is used in amounts sufficient to cause the DNA to become insoluble in solution. Examples of a condensing agent are CTAB, spermine, and spermidine. The magnetic beads may also be added at any stage of the isolation. Even though silica-coated magnetic beads are used as described above, a host of other commercially available beads with different coatings may be used to isolate DNA instead of silica-coated magnetic beads because the magnetic core of the beads eases the isolation of DNA. The DNA can be condensed on to non-magnetic beads as well, in which case the isolation of the beads with the DNA would require routine changes in the protocol, for example, addition of a filtration or centrifugation step. The condensing agent may be present in a buffer and referred to as a condensing buffer. Such buffer refers to a formulation, typically containing CTAB and low salt concentrations, that causes DNA condensation. The DNA may be condensed on a suitable surface such as magnetic beads to allow its isolation from the sample matrix.

The magnetic beads act as a “binding substrate” that provides a surface onto which nucleic acid molecules can bind to. The use of the lysis buffer in conjunction with CTAB and a binding substrate eliminates the need for organic extraction and centrifugation steps for the isolation of DNA.

The general protocol for the isolation of DNA using CTAB can be modified depending on the type of sample and composition of the lysis buffer, fragmentation buffer, lysis-fragmentation buffer, as well as the conditions for condensation and isolation of the condensed DNA. Thus, optimization of the conditions can lead to simplification of the entire procedure. Moreover, further simplification can be achieved by automation.

The shelf-life of the lysis, fragmentation and lysis-fragmentation buffers can be increased by formulating them as solids. Thus, in one embodiment, lysis, fragmentation or lysis-fragmentation buffers are added to a sample as solids, or a sample is added to a tube containing the solid lysis, fragmentation or lysis-fragmentation buffers, followed by incubation steps.

The isolation of DNA from intact cells such as WBC (white blood cells) requires an additional step of DNA fragmentation prior to the PCR-less SNP assay. Random fragmentation of the isolated genomic DNA to yield about 500 to 2000 bp fragments may be carried out by either mechanical or chemical means. Fragmentation is required for optimal hybridization kinetics and good SNP discrimination in the PCR-less SNP assays. The fragmentation step may also act to further purify the DNA by separating and eliminating DNA-associated elements, for example, proteins, polysaccharides, or lipids, making the DNA more available for hybridization in subsequent assays.

In one embodiment, the fragmentation step is not required for DNA isolation from buccal swab samples and mouthwash samples when the lysis buffer also comprises CTAB. Agarose gel analysis shows that a significant proportion of the recovered DNA consists of fragments that are about 200-2000 bp, a size that is optimal for subsequent analysis using PCR-less assays. In some instances, enrichment in shorter fragments of DNA may be optionally performed by including in the lysis step a fragmentation step. Finally, the purification method may also provide cleaner DNA by better eliminating DNA binding elements, such as proteins. The end result is that DNA obtained from a CTAB-based purification method can be used directly in the PCR-less assays without an additional fragmentation step, thus significantly simplifying the overall assay by eliminating labor intensive steps.

In another embodiment, genomic DNA may also be isolated successfully from mouthwash samples. Briefly, a sample is generated using 10 mL water that donors swish in their mouths for 60-90 seconds. About 1 mL of the sample is treated with the lysis buffer in combination with the CTAB and low salt DNA isolation as was used for the buccal swab samples. The DNA fragment size is similar to those obtained for the buccal swab samples. The amount of DNA recovered is sufficient for PCR-less SNP detection. Isolation of DNA using the lysis buffer together with CTAB is also well suited for isolating trans-renal DNA that can be used in PCR-less SNP assays. The concentration of DNA in trans-renal samples is extremely low compared to other types of samples. Thus, isolation of DNA from renal samples further demonstrates the advantage of using a lysis buffer together with CTAB for isolating DNA from samples that have low DNA concentration.

In another embodiment, the lysis buffer together with CTAB is used to isolate DNA from other sources, for example, but not limited to, blood, saliva, mouthwash, sample, and tissue.

In another embodiment, a lysis buffer together with CTAB is used to successfully isolate small amounts of DNA in complex samples with relatively high concentrations of polysaccharides, lipids and other contaminants. DNA isolation from complex samples is particularly difficult because polysaccharides and related contaminants co-purify with DNA. However, a lysis buffer together with CTAB can be used to successfully isolate DNA from complex samples derived from bacteria and other organisms. The lysis buffer and CTAB work efficiently to isolate even extremely low amounts of DNA in a complex background attesting to the general applicability of the procedure.

In one embodiment, magnetic beads, typically coated with a silica layer, are used to provide a surface on which the condensed DNA is allowed to bind or precipitate. The magnetic beads offer a convenient way of isolation using magnets that eliminate centrifugation steps. In yet another embodiment, the DNA is allowed to condense on to other kinds of surfaces, such as but not limited to silica beads, non-magnetic beads, polymeric materials, cross-linked polymeric materials, silica-coated nanoparticles, negatively-charged polymer-coated nanoparticles where the polymer is preferably polyacrylic acid, silica surfaces, and metallic surfaces coated with negatively-charged polymers. Polymeric materials and nanoparticles can be used in a column format and samples containing condensed nucleic acid molecules can be poured over the column. After the condensed DNA binds to the column, elimination of interferents is performed by washing under conditions that do not cause the lost of the bound DNA. The bound DNA is eluted from the column into a small volume, thus purifying and concentrating the DNA in a single step. The silica surfaces and functionalized metallic surfaces likewise would trap the condensed DNA and be released after washing away unbound materials.

In one embodiment, the invention provides a method for detecting a target nucleic acid sequence in a sample, wherein the sample comprises nucleic acid molecules of higher biological complexity than that of amplified nucleic acid molecules and the target nucleic acid sequence differs from a known nucleic acid sequence by at least a single nucleotide, the method comprising the steps of: a) admixing a sample to a lysis buffer, wherein the lysis buffer comprise at least one detergent; b) fragmenting the nucleic acids molecules of step (a); c) condensing the fragmented nucleic acid molecules onto a binding substrate in the presence of CTAB and NaCl so as to bind the nucleic acid molecules onto a surface of the substrate; d) washing the binding substrate having the bound nucleic acid molecules; e) eluting the bound nucleic acid molecules from the binding substrate; f) providing an addressable substrate having a capture oligonucleotide bound thereto, wherein the capture oligonucleotide can recognize at least part of a first portion of the target nucleic acid sequence; g) providing a detection probe comprising detector oligonucleotides, wherein the detector oligonucleotides can hybridize with at least part of a second portion of the target nucleic acid sequence of step (f); h) contacting the nucleic acid molecules of step (e) with the substrate and the detection probe under conditions that are effective for the specific and selective hybridization of the capture oligonucleotide to the first portion of the target nucleic acid sequence and the specific and selective hybridization of the detection probe to the second portion of the target nucleic acid sequence; and i) detecting whether the capture oligonucleotide and detection probe hybridized with the first and second portions of the target nucleic acid sequence. In another embodiment, the addressable substrate has a plurality of capture oligonucleotides bound thereto that can recognize multiple portions of the target nucleic acid sequence and one or more detector probes comprising detector oligonucleotides that can hybridize with one or more portions of the target nucleic acid sequence that are not recognized by the capture oligonucleotides.

In another embodiment, the invention provides a method for identifying a single nucleotide polymorphism in a sample, wherein the sample comprises nucleic acid molecules of higher biological complexity than that of amplified nucleic acid molecules, the method comprising the steps of: a) admixing a sample to a lysis buffer, wherein the lysis buffer comprise at least one detergent; b) fragmenting the nucleic acids molecules of step (a); c) condensing the fragmented nucleic acid molecules onto a binding substrate in the presence of CTAB and NaCl so as to bind the nucleic acid molecules onto a surface of the substrate; d) washing the binding substrate having the bound nucleic acid molecules; e) eluting the bound nucleic acid molecules from the binding substrate; f) providing an addressable substrate having at least one capture oligonucleotide bound thereto, wherein the at least one capture oligonucleotide can recognize a nucleic acid target that comprises a specific polymorphism; g) providing a detector probe having detector oligonucleotides bound thereto, wherein the detector oligonucleotides can hybridize with at least a portion of the nucleic acid target of step (f); h) contacting the nucleic acid molecules of step (e) with the substrate and the detector probe under conditions that are effective for the specific and selective hybridization of the capture oligonucleotide to the nucleic acid target and the hybridization of the detector probe to the nucleic acid target; and i) detecting whether the capture oligonucleotide and detector probe hybridized with the nucleic acid target. In another embodiment, the addressable substrate has a plurality of capture oligonucleotides bound thereto that can recognize multiple portions of the target nucleic acid sequence and the detector probe comprises detector oligonucleotides that can hybridize with a portion of the target nucleic acid sequence that is not recognized by the capture oligonucleotides.

The methods of the invention can discriminate between two sequences that differ by as little as one nucleotide. Thus, in a particular embodiment, the methods of the invention can be used to detect a specific target nucleic acid molecule that has a mutation of at least one nucleotide. In a preferred embodiment, the mutation is a single nucleotide polymorphism (SNP).

In another embodiment, a detector oligonucleotide can be detectably labeled. Various methods of labeling polynucleotides are known in the art and may be used advantageously in the methods disclosed herein. In a particular embodiment, a detectable label of the invention can be fluorescent, luminescent, Raman active, phosphorescent, radioactive, or efficient in scattering light, have a unique mass, or other has some other easily and specifically detectable physical or chemical property, and in order to enhance said detectable property the label can be aggregated or can be attached in one or more copies to a carrier, such as a dendrimer, a molecular aggregate, a quantum dot, or a bead. The label can allow for detection, for example, by photonic, electronic, acoustic, opto-acoustic, gravity, electro-chemical, enzymatic, chemical, Raman, or mass-spectrometric means.

In one embodiment, a detector probe of the invention can be a nanoparticle probe having detector oligonucleotides bound thereto. Nanoparticles have been a subject of intense interest owing to their unique physical and chemical properties that stem from their size. Due to these properties, nanoparticles offer a promising pathway for the development of new types of biological sensors that are more sensitive, more specific, and more cost effective than conventional detection methods. Methods for synthesizing nanoparticles and methodologies for studying their resulting properties have been widely developed over the past 10 years (Klabunde, editor, Nanoscale Materials in Chemistry, Wiley Interscience, 2001). However, their use in biological sensing has been limited by the lack of robust methods for functionalizing nanoparticles with biological molecules of interest due to the inherent incompatibilities of these two disparate materials. A highly effective method for functionalizing nanoparticles with modified oligonucleotides has been developed. See U.S. Pat. Nos. 6,361,944 and 6,417,340 (assignee: Nanosphere, Inc.), which are incorporated by reference in their entirety. The process leads to nanoparticles that are heavily functionalized with oligonucleotides, which have surprising particle stability and hybridization properties. The resulting DNA-modified particles have also proven to be very robust as evidenced by their stability in solutions containing elevated electrolyte concentrations, stability towards centrifugation or freezing, and thermal stability when repeatedly heated and cooled. This loading process also is controllable and adaptable. Nanoparticles of differing size and composition have been functionalized, and the loading of oligonucleotide recognition sequences onto the nanoparticle can be controlled via the loading process. Suitable, but non-limiting examples of nanoparticles include those described U.S. Pat. No. 6,506,564; International Patent Application No. PCT/US02/16382; U.S. patent application Ser. No. 10/431,341 filed May 7, 2003; and International Patent Application No. PCT/US03/14100; all of which are hereby incorporated by reference in their entirety.

The aforementioned loading method for preparing DNA-modified nanoparticles, particularly DNA-modified gold nanoparticle probes, has led to the development of a new colorimetric sensing scheme for oligonucleotides. This method is based on the hybridization of two gold nanoparticle probes to two distinct regions of a DNA target of interest. Since each of the probes are functionalized with multiple oligonucleotides bearing the same sequence, the binding of the target results in the formation of target DNA/gold nanoparticle probe aggregate when sufficient target is present. The DNA target recognition results in a colorimetric transition due to the decrease in interparticle distance of the particles. This colorimetric change can be monitored optically, with a UV-vis spectrophotometer, or visually with the naked eye. In addition, the color is intensified when the solutions are concentrated onto a membrane. Therefore, a simple calorimetric transition provides evidence for the presence or absence of a specific DNA sequence. Using this assay, femtomole quantities and nanomolar concentrations of model DNA targets and polymerase chain reaction (PCR) amplified nucleic acid sequences have been detected. Importantly, it has been demonstrated that gold probe/DNA target complexes exhibit extremely sharp melting transitions which makes them highly specific labels for DNA targets. In a model system, one base insertions, deletions, or mismatches were easily detectable via the spot test based on color and temperature, or by monitoring the melting transitions of the aggregates spectrophotometrically (Storhoff et. al, J. Am. Chem. Soc., 120, 1959 (1998). See also, for instance, U.S. Pat. No. 5,506,564.

Due to the sharp melting transitions, the perfectly matched target could be detected even in the presence of the mismatched targets when the hybridization and detection was performed under extremely high stringency (e.g., a single degree below the melting temperature of the perfect probe/target match). It is important to note that with broader melting transitions such as those observed with molecular fluorophore labels, hybridization and detection at a temperature close to the melting temperature would result in significant loss of signal due to partial melting of the probe/target complex leading to lower sensitivity, and also partial hybridization of the mismatched probe/target complexes leading to lower specificity due to mismatched probe signal. Therefore, nanoparticle probes offer higher specificity detection for nucleic acid detection method.

As described herein, nanoparticle probes, particularly gold nanoparticle probes, are surprising and unexpectedly suited for direct SNP detection with genomic DNA and without amplification. First, the extremely sharp melting transitions observed in nanoparticle oligonucleotide detection probe translate to a surprising and unprecedented assay specificity that could allow single base discrimination even in a human genomic DNA background. Second, a silver-based signal amplification procedure in a DNA microarray-based assay can further provide ultra-high sensitivity enhancement. A nanoparticle or the silver-amplified gold nanoparticle can be detected in a method of the invention, for example, by using an optical device that measures scatter from the nanoparticle or the silver-amplified gold nanoparticle. The optical device may contain the hardware and software to image and provide quantitative measure of the amount of nucleic acid detected. The device may be linked to a computer loaded with software capable of providing a quantitative measure of the amount of nucleic acid detected. For example, a scanner can be linked to a computer loaded with software capable of calculating grayscale measurements, and the grayscale measurements are calculated to provide a quantitative measure of the amount of nucleic acid detected.

The software can also provide a color number for colored spots and can generate images (e.g., printouts) of the scans, which can be reviewed to provide a qualitative determination of the presence of a nucleic acid, the quantity of a nucleic acid, or both. In addition, it has been found that the sensitivity of assays can be increased by subtracting the color that represents a negative result from the color that represents a positive result.

The computer can be a standard personal computer, which is readily available commercially. Thus, the use of a standard scanner linked to a standard computer loaded with standard software can provide a convenient, easy, inexpensive means of detecting and quantitating nucleic acids when the assays are performed on substrates. The scans can also be stored in the computer to maintain a record of the results for further reference or use. Of course, more sophisticated instruments and software can be used, if desired.

Silver staining can be employed with any type of nanoparticles that catalyze the reduction of silver. Preferred are nanoparticles made of noble metals (e.g., gold and silver). See Bassell, et al., J. Cell Biol., 126, 863-876 (1994); Braun-Howland et al., Biotechniques, 13, 928-931 (1992). If the nanoparticles being employed for the detection of a nucleic acid do not catalyze the reduction of silver, then silver ions can be complexed to the nucleic acid to catalyze the reduction. See Braun et al., Nature, 391, 775 (1998). Also, silver stains are known which can react with the phosphate groups on nucleic acids.

Silver staining can be used to produce or enhance a detectable change in any assay performed on a substrate, including those described above. In particular, silver staining has been found to provide a huge increase in sensitivity for assays employing a single type of nanoparticle so that the use of layers of nanoparticles, aggregate probes and core probes can often be eliminated.

In another embodiment, oligonucleotides attached to a substrate can be located between two electrodes, the nanoparticles can be made of a material that is a conductor of electricity, and step (d) in the methods of the invention can comprise detecting a change in conductivity. In yet another embodiment, a plurality of oligonucleotides, each of which can recognize a different target nucleic acid sequence, are attached to a substrate in an array of spots and each spot of oligonucleotides is located between two electrodes, the nanoparticles are made of a material that is a conductor of electricity, and step (d) in the methods of the invention comprises detecting a change in conductivity. The electrodes can be made, for example, of gold and the nanoparticles are made of gold. Alternatively, a substrate can be contacted with silver stain to produce a change in conductivity.

In a particular embodiment, nucleic acid molecules in a sample are of higher biological complexity than amplified nucleic acid molecules. One of skill in the art can readily determine the biological complexity of a target nucleic acid sequence using methods as described, for example, in Lewin, GENE EXPRESSION 2, Second Edition: Eukaryotic Chromosomes, 1980, John Wiley & Sons, New York, which is hereby incorporated by reference.

Hybridization kinetics are absolutely dependent on the concentration of the reaction partners, i.e. the strands that have to hybridize. In a given quantity of DNA that has been extracted from a cell sample, the amount of total genomic, mitochondrial (if present), and extra-chromosomal elements (if present) DNA is only a few micrograms. Thus, the actual concentrations of the reaction partners that are to hybridize will depend on the size of these reaction partners and the complexity of the extracted DNA. For example, a target sequence of 30 bases that is present in one copy per single genome is present in different concentrations when comparing samples of DNA from different sources and with different complexities. For example, the concentration of the same target sequence in 1 microgram of total human DNA is about 1000 fold lower than in a 1 microgram bacterial DNA sample, and it would be about 1,000,000 fold lower than in a sample consisting in 1 microgram of a small plasmid DNA.

The high complexity (1×10⁹ nucleotides) of the human genome demands an extraordinary high degree of specificity because of redundancies and similar sequences in genomic DNA. For example, to differentiate a capture strand with 25meric oligonuclotides from the whole human genome requires a degree of specificity with discrimination ability of 40,000,000:1. In addition, since the wild type and mutant targets differ only by one base in 25mer capture sequence, it requires distinguishing two targets with 96% homology for successful genotyping. The methods of the invention surprisingly and unexpectedly provide efficient, specific and sensitive detection of a target nucleic acid molecule having high complexity compared with amplified nucleic acid molecules.

The biological complexity of target nucleic acid molecules in a sample derived from human tissues is on the order of 1,000,000,000, but may be up to 10 fold higher or lower for genomes from plants or animals. Preferably, the biological complexity is about 50,000 to 5,000,000,000. Most preferably, the biological complexity is about 1,000,000,000.

In one embodiment, the hybridization conditions are effective for the specific and selective hybridization, whereby single base mismatches are detectable, of the capture oligonucleotide and/or the detector oligonucleotides to the target nucleic acid sequence, even when said target nucleic acid is part of a nucleic acid sample with a biological complexity of 50,000 or larger, as shown, for example, in the Examples below.

The methods of the invention can further be used for identifying specific species of a biological microorganism (e.g. Staphylococcus, Bacillus anthracis, Bacillus thuringensis, Yersinia pestis, Francisella tularensis, and Vaccinia) and/or for detecting genes that confer antibiotic resistance (e.g. mecA gene which confers resistance to the antibiotic methicillin). Many other species of microorganism may be detected by the methods of the invention, as well as their characteristic genes. Such organisms include, but not limited to, bacteria, fungi, viruses, and protozoans.

Methicillin resistant strains of Staphylococcus aureus (MRSA) have become first ranking nosocomial pathogens worldwide. These bacteria are responsible for over 40% of all hospital-born staphylococcal infections in large teaching hospitals in the United States. Most recently they have become prevalent in smaller hospitals (20% incidence in hospitals with 200 to 500 beds), as well as in nursing homes (Wenzel et al., 1992, Am. J. Med. 91(Supp 3B):221-7). An unusual and most unfortunate property of MRSA strains is their ability to pick up additional resistance factors which suppress the susceptibility of these strains to other, chemotherapeutically useful antibiotics. Such multi-resistant strains of bacteria are now prevalent all over the world and the most “advanced” forms of these pathogens carry resistance mechanisms to most of the usable antibacterial agents (Blumberg et al., 1991, J. Inf. Disease, Vol. 63, pp. 1279-85).

The central genetic element of methicillin resistance is the so called mecA gene. This gene is found on a piece of DNA of unknown, non-staphylococcal origin that the ancestral MRSA cell(s) must have acquired from a foreign source. The mecA gene encodes for a penicillin binding protein (PBP) called PBP2A (Murakami and Tomasz, 1989, J. Bacteriol. Vol. 171, pp. 874-79), which has very low affinity for the entire family of beta lactam antibiotics. In the current view, PBP2A is a kind of “surrogate” cell wall synthesizing enzyme that can take over the vital task of cell wall synthesis in staphylococci when the normal complement of PBPs (the normal catalysts of wall synthesis) can no longer function because they have become fully inactivated by beta lactam antibiotic in the environment. The critical nature of the mecA gene and its gene product PBP2A for the antibiotic resistant phenotype was demonstrated by early transposon inactivation experiments in which the transposon Tn551 was maneuvered into the mecA gene. The result was a dramatic drop in resistance level from the minimum inhibitory concentration (MIC) value of 1600 ug/ml in the parental bacterium to the low value of about 4 ug/ml in the transposon mutant (Matthews and Tomasz, 1990, Antimicrobial Agents and Chemotherapy, Vol. 34, pp. 1777-9).

Staphylococcal infections acquired in hospital have become increasingly difficult to treat with the rise of antibiotic resistant strains, and the increasing number of infections caused by coagulase negative Staphylococcal species. Effective treatment of these infections is diminished by the lengthy time many tests require for the determination of species identification (speciation) and antibiotic resistance. With the rapid identification of both species and antibiotic resistance status, the course of patient treatment can be implemented earlier and with less use of broad-spectrum antibiotics. Accordingly, there is an apparent need for a rapid, highly sensitive and selective method for identifying and distinguishing Staphylococci species/or and for mecA gene detection.

In another embodiment, the invention provides oligonucleotide sequences and their reverse complements for Staphylococcal speciation and/or methicillin resistance gene (mecA) detection, nanoparticle-labeled probes, methods, and kits that employ these sequences. These sequences have been designed to be highly sensitive as well as selective for Staphylococcal species or the mecA gene, which gives rise to some forms of antibiotic resistance. These sequences can be used for the intended purpose of mecA gene detection or Staphylococcal speciation as well as negative controls for other systems. Currently, S. aureus can be differentiated from S. epidermidis using either sets probes Tuf 3 and 4, or Tuf 5 and 6 in combination with probe Tuf 2 as shown in the Examples below. Sequences labeled 16S are used to detect the presence of 16S rRNA or DNA contained within the genus Staphylococcus. Conventional methods such as standard phosphoramidite chemistry can be used to create these sequences both as capture probes and/or as nanoparticle labeled probes.

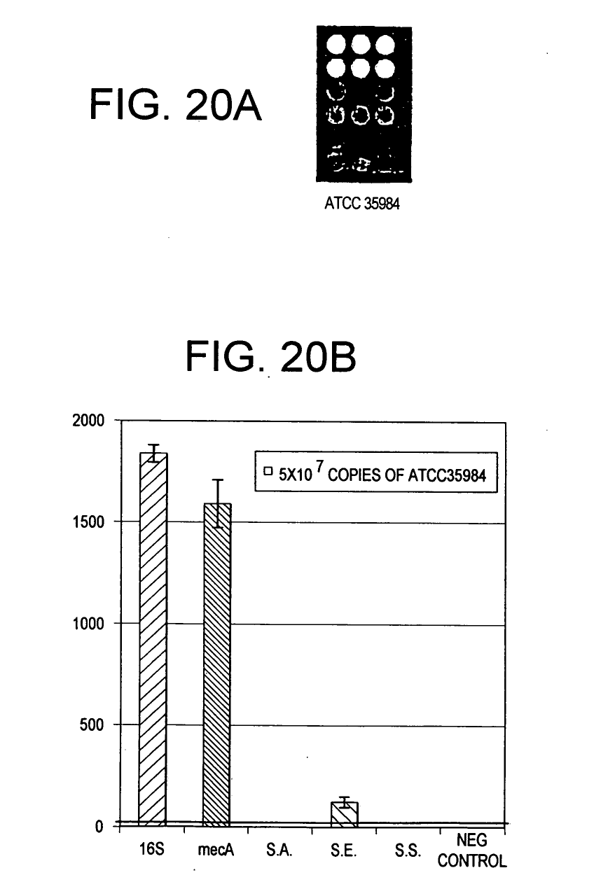

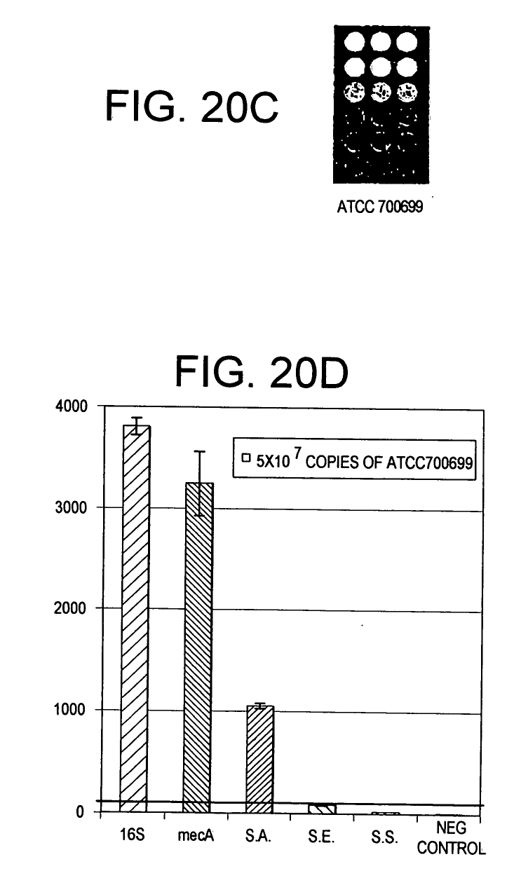

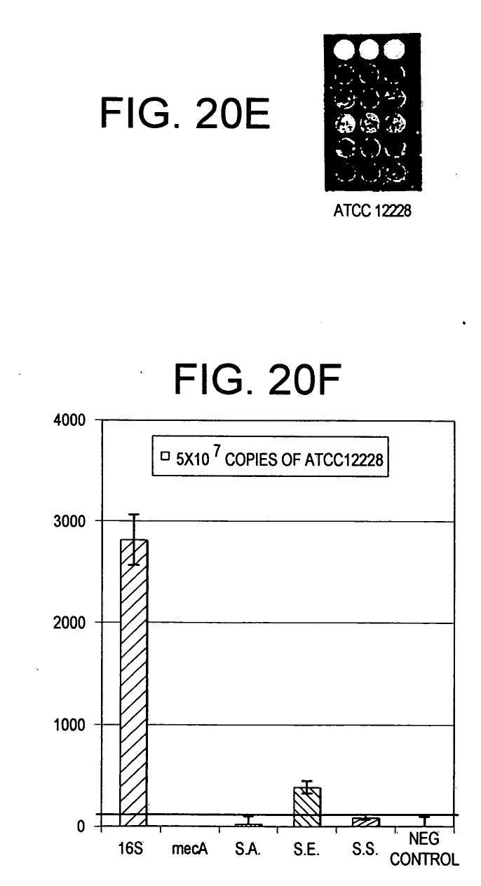

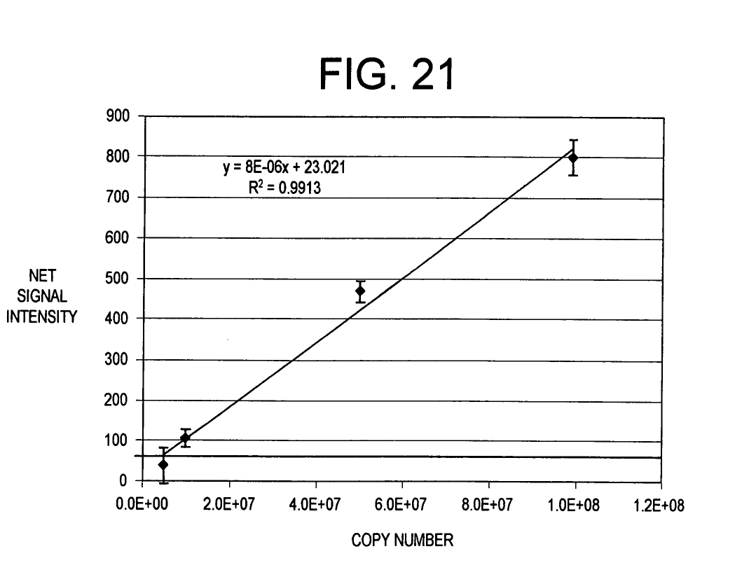

In another embodiment of the invention, the sequences can be used in a method for staphylococcal speciation and/or mecA detection with unamplified genomic DNA. The mecA gene sequences of the invention have been used to detect as little as 1×10⁻¹³ M (100 fM, 3×10⁶ copies) double stranded PCR products and 33 ng (1×10⁷ copies) of sonicated total genomic DNA in a 50 μl reaction for the mecA gene under the current assay conditions and format (see FIG. 21). The sensitivity and specificity of the Tuf gene sequences used for speciation of S. aureus and S. epidermidis also were tested in the assay using PCR amplified gene products or total bacterial genomic DNA. The current lower limit of detection was determined to be 1×10⁻¹² M (1 pM, or 3×10⁷ copies) of double stranded PCR products and 150 ng (5×10⁷ copies) of sonicated genomic DNA in a 50 μl reaction (see FIG. 20). The conditions under which these assays were performed are described below. The methods of the invention surprisingly provide efficient, sensitive, and specific detection of different species of Staphylococcus by distinguishing DNA sequences that differ by a single base-pair or greater, using bacterial genomic DNA without prior complexity reduction or target amplification.

In yet another embodiment of the invention, when using PCR amplicons, a second nanoparticle probe can be used in place of the capture sequence attached to the array substrate as described in PCT/US01/46418 (Nanosphere, Inc., Assignee), which is incorporated by reference in its entirety. This system can be detected optically (e.g. color or light scattering) when target DNA hybridizes to both nanoparticle probes, which leads to a color change. This type of assay can be used for the purpose of Staphylococcus species identification or mecA gene detection as described for the above assays.

### EXAMPLES

The invention is demonstrated further by the following illustrative examples. The examples are offered by way of illustration and are not intended to limit the invention in any manner. In these examples all percentages are by weight if for solids and by volume if for liquids, and all temperatures are in degrees Celsius unless otherwise noted.

### Example 1

Single-Step and Two-Step Hybridization Methods for Identifying SNPs in Unamplified Genomic DNA using Nanoparticle Probes

Gold nanoparticle-oligonucleotide probes to detect the target factor II, MTHFR and factor V sequences were prepared using procedures described in PCT/US97/12783, filed Jul. 21, 1997; PCT/US00/17507, filed Jun. 26, 2000; PCT/US01/01190, filed Jan. 12, 2001, which are incorporated by reference in their entirety. FIG. 3 illustrates conceptually the use of gold nanoparticle probes having oligonucleotides bound thereto for detection of target DNA using a DNA microarray having wild type or mutant capture probe oligonucleotides. The sequence of the oligonucleotides bound to the nanoparticles are complementary to one portion of the sequence of target while the sequence of the capture oligonucleotides bound to the glass chip are complementary to another portion of the target sequence. Under hybridization conditions, the nanoparticle probes, the capture probes, and the target sequence bind to form a complex. Signal detection of the resulting complex can be enhanced with conventional silver staining.

(a) Preparation Of Gold Nanoparticles

Gold colloids (13 nm diameter) were prepared by reduction of HAuCl₄ with citrate as described in Frens, 1973, Nature Phys. Sci., 241:20 and Grabar, 1995, Anal. Chem. 67:735. Briefly, all glassware was cleaned in aqua regia (3 parts HCl, 1 part HNO₃), rinsed with Nanopure H₂O, then oven dried prior to use. HAuCl₄ and sodium citrate were purchased from Aldrich Chemical Company. Aqueous HAuCl₄ (1 mM, 500 mL) was brought to reflux while stirring. Then, 38.8 mM sodium citrate (50 mL) was added quickly. The solution color changed from pale yellow to burgundy, and refluxing was continued for 15 min. After cooling to room temperature, the red solution was filtered through a Micron Separations Inc. 1 micron filter. Au colloids were characterized by UV-vis spectroscopy using a Hewlett Packard 8452A diode array spectrophotometer and by Transmission Electron Microscopy (TEM) using a Hitachi 8100 transmission electron microscope. Gold particles with diameters of 15 nm will produce a visible color change when aggregated with target and probe oligonucleotide sequences in the 10-35 nucleotide range.

(b) Synthesis Of Oligonucleotides

The capture probe oligonucleotides complementary to segments of the MTHFR, factor II or factor V DNA sequence were synthesized on a 1 micromole scale using a ABI 8909 DNA synthesizer in single column mode using phosphoramidite chemistry [Eckstein, F. (ed.) Oligonucleotides and Analogues. A Practical Approach (IRL Press, Oxford, 1991)]. The capture sequences contained either a 3′-amino modifier that serves as the active group for covalent attachment during the arraying process. The oligonucleotides were synthesized by following standard protocols for DNA synthesis. Columns with the 3′-amino modifier attached to the solid support, the standard nucleotide phosphoramidites and reagents were obtained from Glen Research. The final dimethoxytrityl (DMT) protecting group was not cleaved from the oligonucleotides to aid in purification. After synthesis, DNA was cleaved from the solid support using aqueous ammonia, resulting in the generation of a DNA molecule containing a free amine at the 3′-end. Reverse phase HPLC was performed with an Agilent 1100 series instrument equipped with a reverse phase column (Vydac) by using 0.03 M Et₃NH⁺ OAc⁻ buffer (TEAA), pH 7, with a 1%/min. gradient of 95% CH₃CN/5% TEAA. The flow rate was 1 mL/min. with UV detection at 260 nm. After collection and evaporation of the buffer, the DMT was cleaved from the oligonucleotides by treatment with 80% acetic acid for 30 min at room temperature. The solution was then evaporated to near dryness, water was added, and the cleaved DMT was extracted from the aqueous oligonucleotide solution using ethyl acetate. The amount of oligonucleotide was determined by absorbance at 260 nm, and final purity assessed by analytical reverse phase HPLC.

The capture sequences employed in the assay for the MTHFR gene are as follows: MTHFR wild-type, 5′ GATGAAATCGGCTCCCGCAGAC-NH₂ 3′ (MTHFR-SNP/Cap6-WT22; SEQ ID NO: 1), and MTHFR mutant, 5′ ATGAAATCGACTCCCGCAGACA-NH₂ 3′ (MTHFR-SNP/Cap7-mut22; SEQ ID NO: 2). The corresponding capture oligonucleotides for the Factor V gene are as follows: Factor V wild type, 5′ TGG ACA GGC GAG GAA TAC AGG TAT-NH₂ 3′ (FV-Cap-WT24; SEQ ID NO: 3) and Factor V mutant, 5′CTG GAC AGG CAA GGA ATA CAG GTA TT-NH₂ 3′ (FV-Cap-mut26; SEQ ID NO: 4). Factor II wild-type: 5′ CTCAGCGAGCCTCAATGCTCCC—NH₂ 3′ (FII-SNP/Cap1-WT22; SEQ ID NO: 5) and Factor II mutant, 5′ CTCTCAGCAAGCCTCAATGCTCC-NH₂ 3′ (FII-SNP/Cap1-mut23; SEQ ID NO: 6).

The detection probe oligonucleotides designed to detect Factor II, MTHFR and Factor V genes comprise a steroid disulfide linker at the 5′-end followed by the recognition sequence. The sequences for the probes are described: FII probe, 5′ Epi-TCC TGG AAC CAA TCC CGT GAA AGA ATT ATT TTT GTG TTT CTA AAA CT 3′ (FII-Pro 1-47; SEQ ID NO: 7), MTHFR probe, 5′Epi-AAA GAT CCC GGG GAC GAT GGG GCA AGT GAT GCC CAT GTC GGT GCA TGC CTT CAC AAA G 3′(MTHFR-Pro 11-58; SEQ ID NO: 8), Factor V probe, 5′ Epi-CCA CAG AAA ATG ATG CCC AGT GCT TAA CAA GAC CAT ACT ACA GTG A 3′ (FV-Pro 46; SEQ ID NO: 9).

The synthesis of the probe oligonucleotides followed the methods described for the capture probes with the following modifications. First, instead of the amino-modifier columns, supports with the appropriate nucleotides reflecting the 3′-end of the recognition sequence were employed. Second, the 5′-terminal steroid-cyclic disulfide was introduced in a coupling step by employing a modified phosphoramidite containing the steroid disulfide (see Letsinger et al., 2000, Bioconjugate Chem. 11:289-291 and PCT/US01/01190 (Nanosphere, Inc.), the disclosure of which is incorporated by reference in its entirety). The phosphoramidite reagent may be prepared as follows: a solution of epiandrosterone (0.5 g), 1,2-dithiane-4,5-diol (0.28 g), and p-toluenesulfonic acid (15 mg) in toluene (30 mL) was refluxed for 7 h under conditions for removal of water (Dean Stark apparatus); then the toluene was removed under reduced pressure and the residue taken up in ethyl acetate. This solution was washed with water, dried over sodium sulfate, and concentrated to a syrupy residue, which on standing overnight in pentane/ether afforded a steroid-dithioketal compound as a white solid (400 mg); Rf (TLC, silica plate, ether as eluent) 0.5; for comparison, Rf values for epiandrosterone and 1,2-dithiane-4,5-diol obtained under the same conditions are 0.4, and 0.3, respectively. Recrystallization from pentane/ether afforded a white powder, mp 110-112° C.; ¹H NMR, δ 3.6 (1H, C³OH), 3.54-3.39 (2H, m 20CH of the dithiane ring), 3.2-3.0 (4H, m 2CH₂S), 2.1-0.7 (29H, m steroid H); mass spectrum (ES⁺) calcd for C₂₃H₃₆O₃S₂ (M+H) 425.2179, found 425.2151. Anal. (C₂₃H₃₇O₃S₂) S: calcd, 15.12; found, 15.26. To prepare the steroid-disulfide ketal phosphoramidite derivative, the steroid-dithioketal (100 mg) was dissolved in THF (3 mL) and cooled in a dry ice alcohol bath. N,N-diisopropylethylamine (80 μL) and β-cyanoethyl chlorodiisopropylphosphoramidite (80 μL) were added successively; then the mixture was warmed to room temperature, stirred for 2 h, mixed with ethyl acetate (100 mL), washed with 5% aq. NaHCO₃ and with water, dried over sodium sulfate, and concentrated to dryness. The residue was taken up in the minimum amount of dichloromethane, precipitated at −70° C. by addition of hexane, and dried under vacuum; yield 100 mg; ³¹P NMR 146.02. After completion of the DNA synthesis, the epiandrosterone-disulfide linked oligonucleotides were deprotected from the support under aqueous ammonia conditions and purified on HPLC using reverse phase column as described above.

(c) Attachment Of Oligonucleotides To Gold Nanoparticles

The probe was prepared by incubating initially a 4 μM solution of the oligonucleotide with a ˜14 nM solution of a 15 nm citrate-stabilized gold nanoparticle colloid solution in a final volume of 2 mL for 24 h. The salt concentration in this preparation was raised gradually to 0.8 M over a period of 40 h at room temperature. The resulting solution was passed through a 0.2 μm cellulose acetate filter and the nanoparticle probe was pelleted by spinning at 13,000 G for 20 min. After removing the supernatant, the pellet was re-suspended in water. In a final step, the probe solution was pelleted again and resuspended in a probe storage buffer (10 mM phos, 100 mM NaCl, 0.01% w/v NaN₃). The concentration was adjusted to 10 nM after estimating the concentration based on the absorbance at 520 nm (ε=2.4×10⁸ M⁻¹ cm⁻¹).

The following nanoparticle-oligonucleotide conjugates specific for factor II, MTHFR and factor V DNA were prepared in this manner:

Factor II Probe:
gold-S′-5′-[TCC TGG AAC CAA TCC CGT GAA AGA ATT
ATT TTT GTG TTT CTA AAA CT-3′]ₙ
(FII-ProI-47; SEQ ID NO:10)
MTHFR Probe:
gold-S′-5′-[AAA GAT CCC GGG GAC GAT GGG GCA AGT
GAT GCC CAT GTC GGT GCA TGC CTT CAC AAA G-3′]ₙ
(MTHFR-II58; SEQ ID NO:11)
Factor V Probe:
gold-S′-5′-[CCA CAG AAA ATG ATG CCC AGT GCT TAA
CAA GAC CAT ACT ACA GTG A-3′]ₙ
(FV-46; SEQ ID NO:12)

S′ indicates a connecting unit prepared via an epiandrosterone disulfide group; n reflect the number of the recognition oligonucleotides.
(d) Preparation of DNA Microarrays

Capture strands were arrayed on Superaldehyde slides (Telechem) or CodeLinke slides (Amersham, Inc.) by using a GMS417 arrayer (Affymetrix). The positioning of the arrayed spots was designed to allow multiple hybridization experiments on each slide, achieved by partitioning the slide into separate test wells by silicone gaskets (Grace Biolabs). The wild type and mutant spots were spotted in triplicate in manufacturer-provided spotting buffers. Protocols recommended by the manufacturer were followed for post-array processing of the slides.

(e) Hybridization

Factor V SNP detection assay procedure The Factor V SNP detection was performed by employing the following protocol. Sonicated human placental DNA, genotyped as homozygous wild-type, or salmon sperm DNA (Sigma) was precipitated with ethanol and dissolved in a 10 nM solution of FV probe solution. Additional components were added to this mixture such that the final hybridization mixture (5×μL) contained 3×SSC, 0.03% Tween 20, 23% formamide, 5 nM FV probe, and 10 μg human DNA, or as indicated. The hybridization mixture was added to the test well after a 4 min, 99° C. heat denaturation step. The arrays were incubated at 50° C. for 90 min. Post-hybridization washes were initiated by immersing arrays for 1 min in 0.5 M NaNO₃, 0.05% Tween 20 at room temperature. The gasket was removed and the test slide was washed again in 0.5M NaNO₃/0.05% Tween 20 solution and incubated at room temperature for 3 min (2×) with gentle agitation. The slides were stained with the silver enhancing solution as described above and dried on a spin dryer and imaged on an ArrayWorx® biochip reader (Model no. AWE, Applied Precision Inc., Issaquah, Wash., U.S.A.).

(f) Results

Factor VSNP Detection

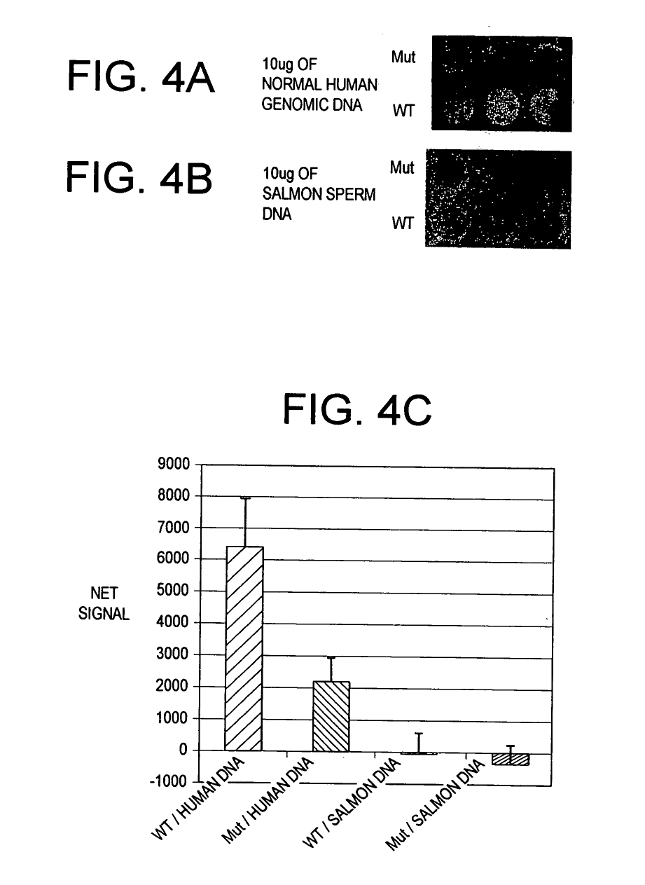

FIG. 4 shows SNP discrimination of Factor V gene in human genomic DNA on Superaldehyde slides. The test array contains wild type and mutant capture spots. The array shown on the top was hybridized with wild-type human genomic DNA while the array on the bottom was hybridized with sonicated salmon sperm DNA. The signal at the wild-type spots is significantly higher than mutant spots with wild-type human genomic DNA hybridization to indicate a Factor V homozygous wild-type genotype. Under the hybridization conditions, no signal is observed for the salmon sperm DNA hybridization and serves as a control in the assay. SNP discrimination was also examined with arrays on CodeLink® slides.

The experiment was designed to show that the hybridization on the wt capture spots was not due to some other sequences, but was specific to a genome that contains the human factor V gene. Using total human wt DNA, the expected high hybridization signal was observed at the wt capture spots, and about 3 fold less signal was observed at the mutant spot. However, when the genomic DNA extracted from salmon sperm was used as target, no signals are observed, since this DNA does not contain the human factor V gene.

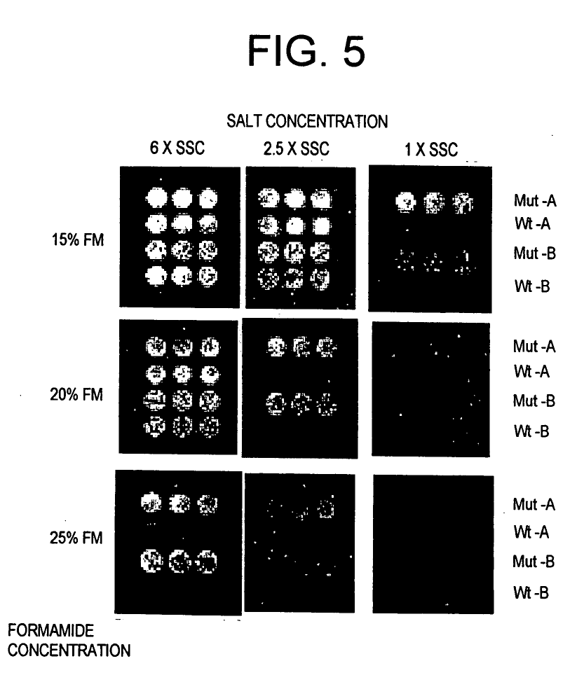

The importance of adjusting the hybridization conditions in order to make this process capable of discriminating between two target nucleic acids that differ by 1 nucleotide (the SNP site) is shown in FIG. 5. An appropriate balance between formamide and SSC buffer salt concentration has to be determined such that the target sequence (in this case from a homozygous patient with a mutation in the Factor V gene) binds preferentially to its cognate capture probe (i.e. the Mut-A or Mut-B sequence). In addition, FIG. 5 shows the effect of various sizes of capture oligonucleotide sequences in hybridization. The Mut-A sequence was 26 nucleotides long, while the Mut-B sequence was 21 nucleotides long. The results demonstrated a significant difference in the specific signal at the condition of 15% FM/1×SSC, but at 25% FM/6×SSC there was no difference and both probes generated a strong signal with good discrimination.

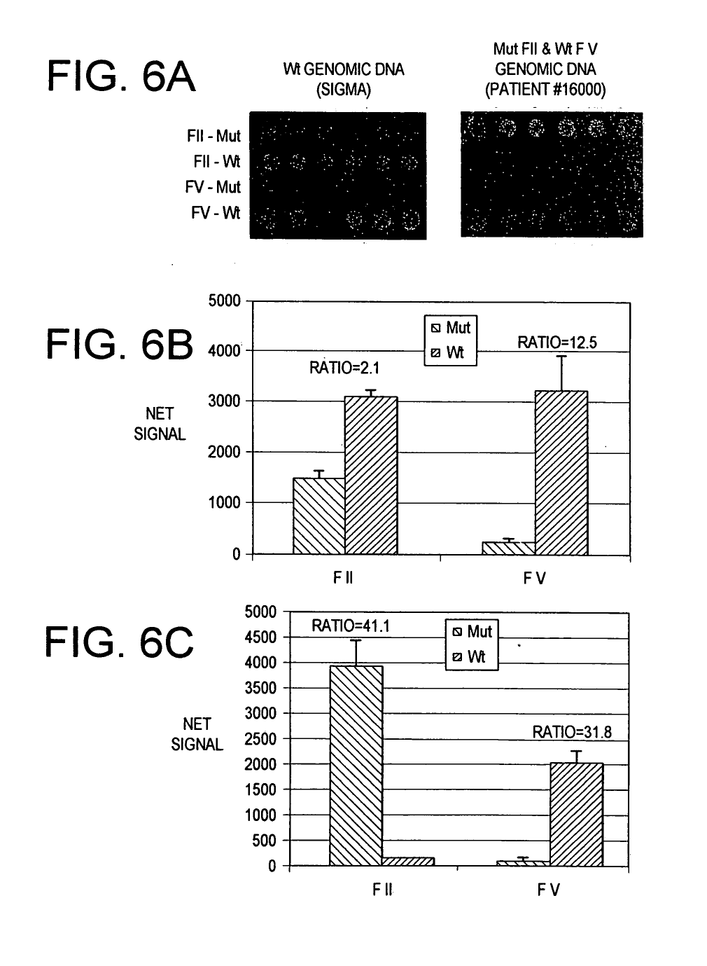

To determine if more than one SNP type could be detected in the same sample under the same conditions, genomic DNA was tested for the presence of wild type and mutant Factor II and Factor V genes. Normal human (wt) genomic DNA, capture oligonucleotides attached to a substrate, and nanoparticle probes were mixed together in 35% FM and 4×SSC at 40° C. for one hour. A signal was generated preferentially at the wt capture spots for both, the Factor II and the Factor V gene (FIG. 6). When using the total genomic DNA from an individual that was homozygous for a mutation in Factor II, but homozygous wt for Factor V, the same array under the same hybridization conditions gave a signal preferentially at the mutant capture spots for Factor II and on the wt capture spots for Factor V, clearly and correctly identifying the genetic make-up of this person with respect to his SNP configuration of these two genes (FIG. 6). The results demonstrate that the capture oligonucleotide sequences and hybridization conditions can be designed so that more than one SNP type can be tested within the same array and under the same hybridization conditions. Also, SNP discrimination is possible between wt and mutant DNA, independent of whether the input DNA is from a normal or a mutant source.

Two-Step Hybridization

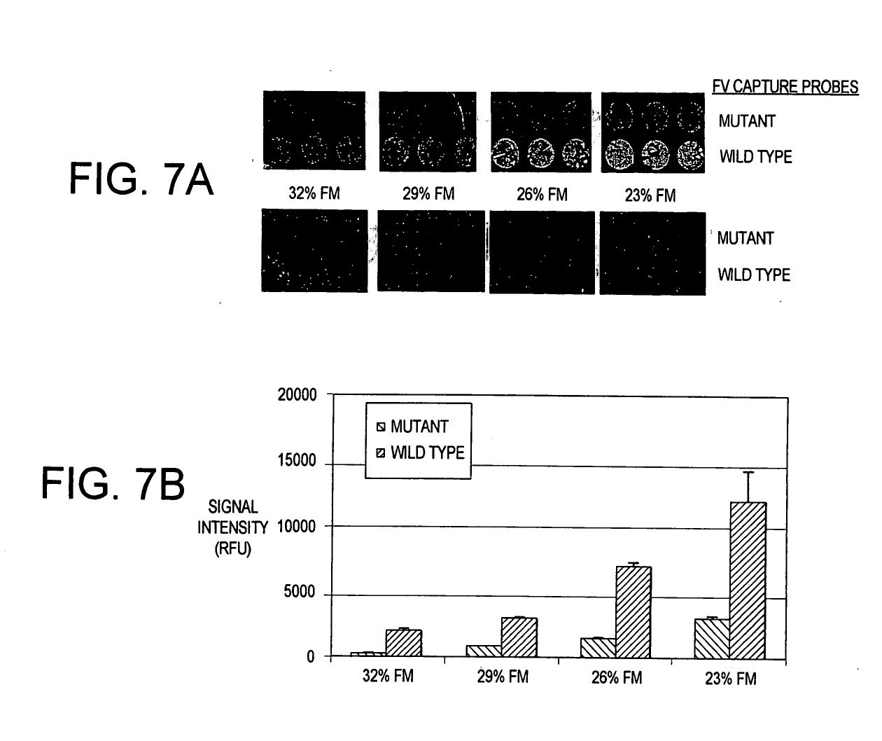

More experiments were conducted to determine the effect of various stringency conditions on SNP discrimination. Test arrays were hybridized at different stringencies by employing different percentages of formamide in the assay (FIG. 7). With increasing stringency there is loss of signal, which translates to improved specificity of the signal. Almost no signal was observed in the no-target controls. Quantitation of the signals from the spots revealed a 3-6—fold higher signal for the wild-type spots over that for the mutant spots (FIG. 7B). Together the results provide support for SNP discrimination in genomic DNA without the need for any target amplification strategies.

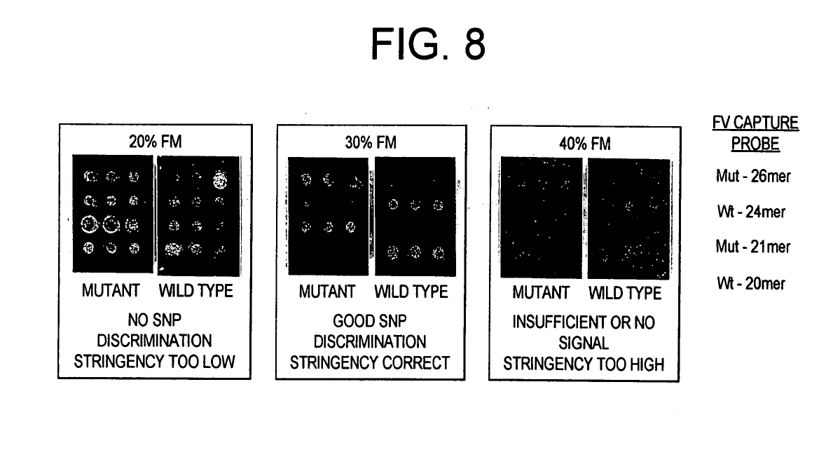

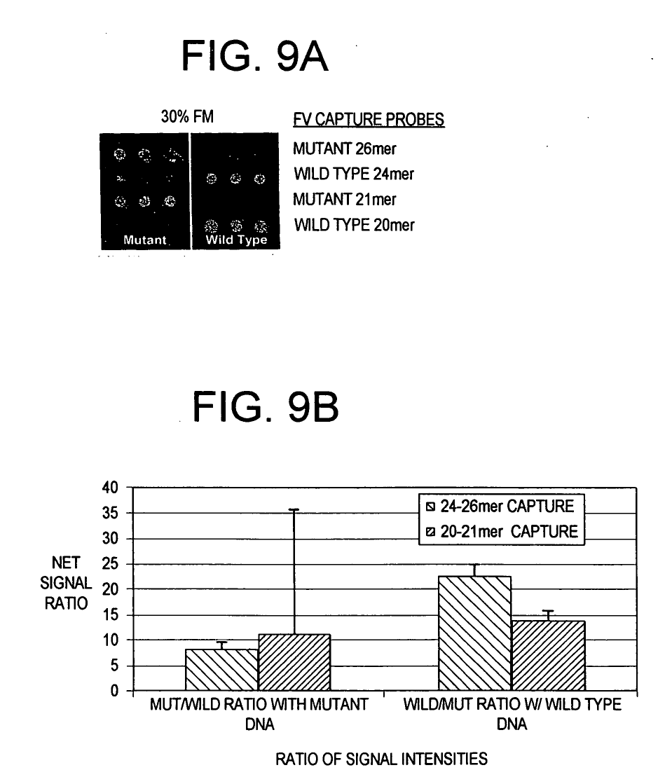

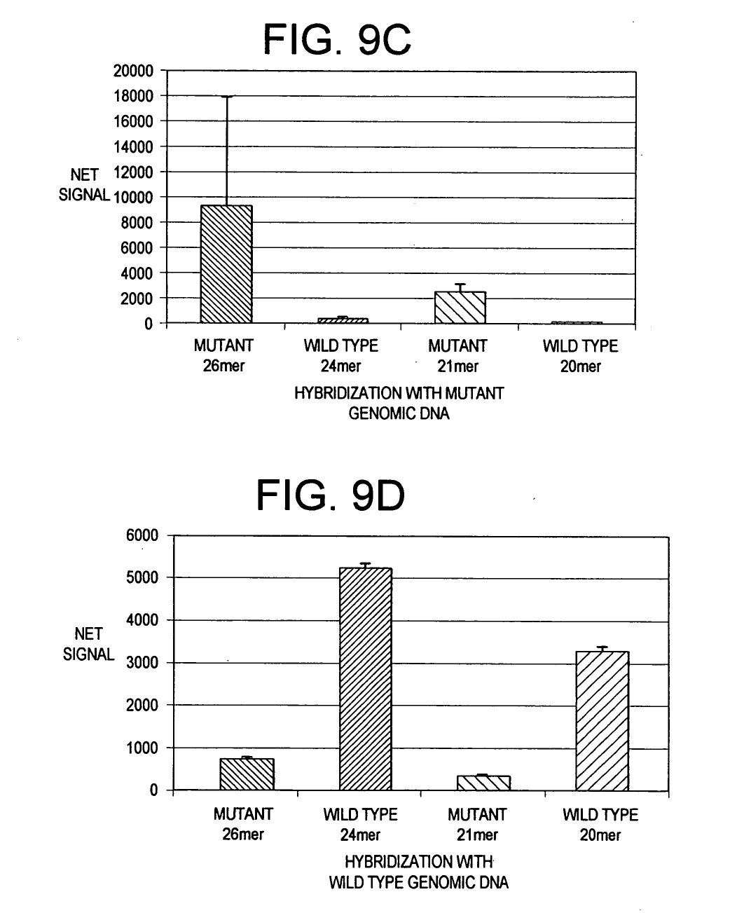

Capture oligonucleotides of various lengths, including 20, 21, 24, or 26 nucleotides (FV-WT20 (SEQ ID NO: 13): 5′(GGACAGGCGAGGAATACAGG)-(PEG)×3-NH₂, 3′ FV-mut21 (SEQ ID NO: 14): 5′(TGGACAGGCAAGGAATACAGG)-(PEG)×3-NH₂ 3′, FV-wt24 (SEQ ID NO: 15): 5′ TGG ACA GGC GAG GAA TAC AGG TAT-NH₂ 3′, FV-mut26 (SEQ ID NO: 16): 5′ CTG GAC AGG CAA GGA ATA CAG GTA TT-NH₂ 3′) were printed on CodeLink slides as described above and were added to 5 μg of normal human placenta genomic DNA (Sigma, St. Louis, Mo.) or factor V mutant human genomic DNA (isolated from repository culture GM14899, factor V deficiency, Coriell Institute). The slides and DNA were incubated in 20% FM, 30% FM, or 40% FM, and 4×SSC/0/04% Tween at 40° C. for 2 hours in the first step. The slides were then washed in 2×SSC at room temperature for 3 minutes. After washing, nanoparticle probes with detection oligonucleotides that recognized Factor V were added and the mixture was then incubated for 1 hour at 40° C. The signal was detected by silver staining as described above. The results showed that under optimally tuned conditions (30% FM in this case), the human wt DNA generated a signal on the wt probes only, while the human mutant DNA generated a signal only at the mutant capture probes (FIG. 8). Changing the stringency conditions resulted in either loss of discrimination (stringency too low) or loss of signal (stringency too high). FIG. 9 shows the quantitative data for the perfect (center) hybridization condition in FIG. 8.

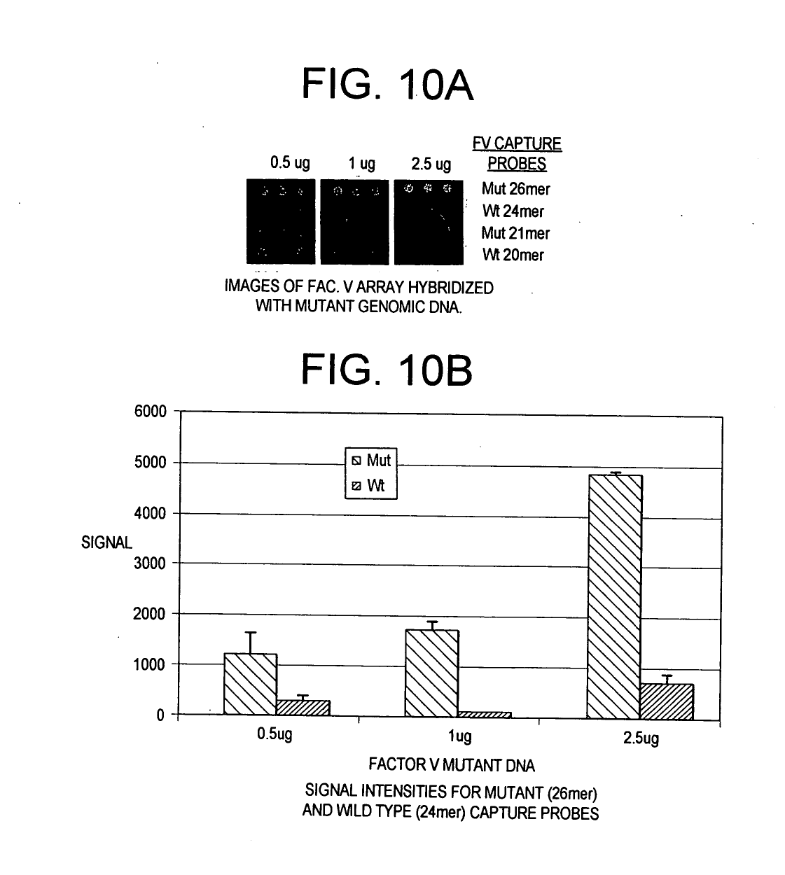

The experiment was then repeated under the optimal conditions with various concentrations of DNA. As seen in FIG. 10, SNP discrimination was successful when the concentration of DNA was 0.5 μg, 1.0 μg, and 2.5 μg. Thus, the method could detect the SNP with very little (less than 1 microgram) total human DNA. These results also demonstrated the importance of capture oligonucleotide design and the appropriate match of the stringency conditions to the length and nucleotide composition of the capture (and detection) probes.

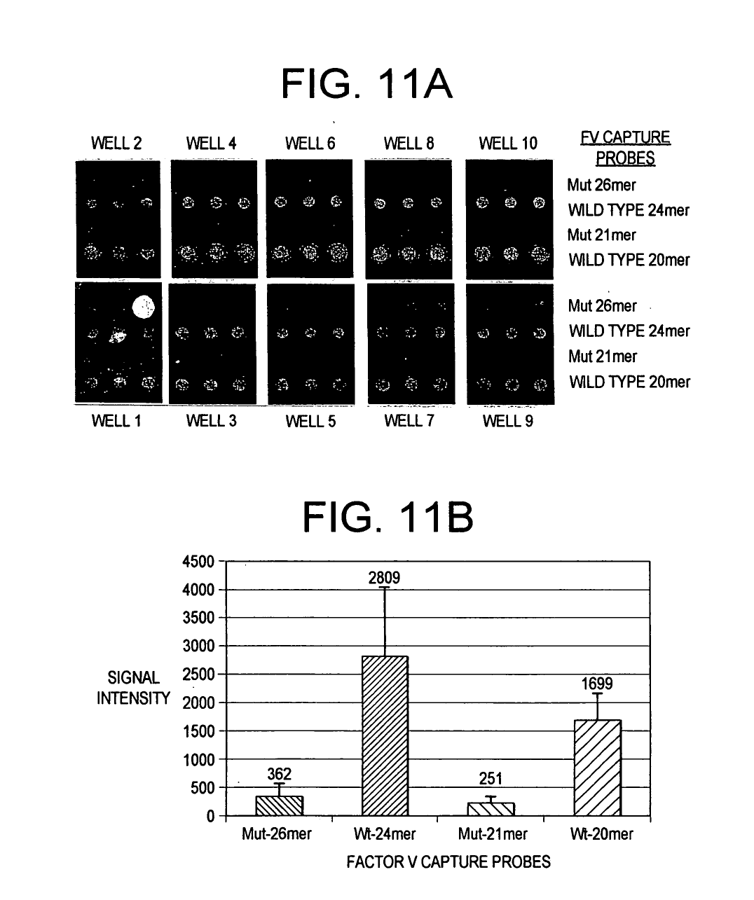

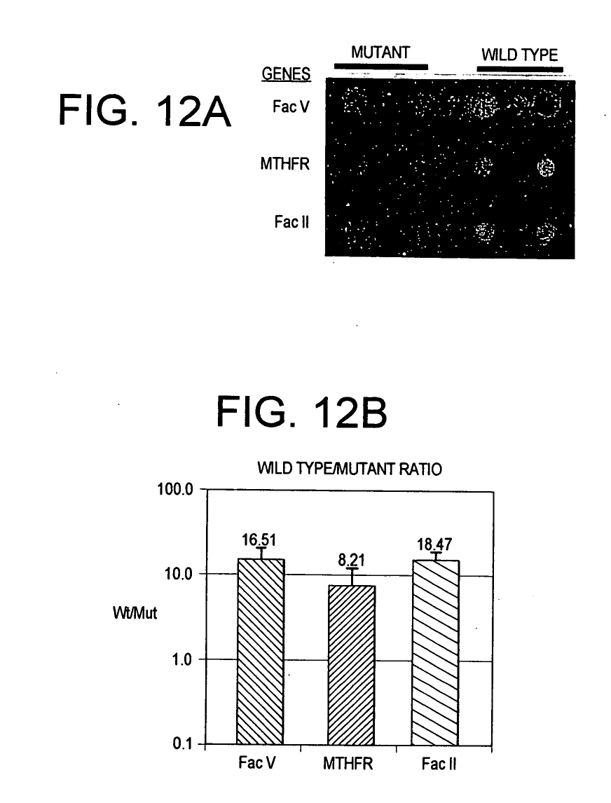

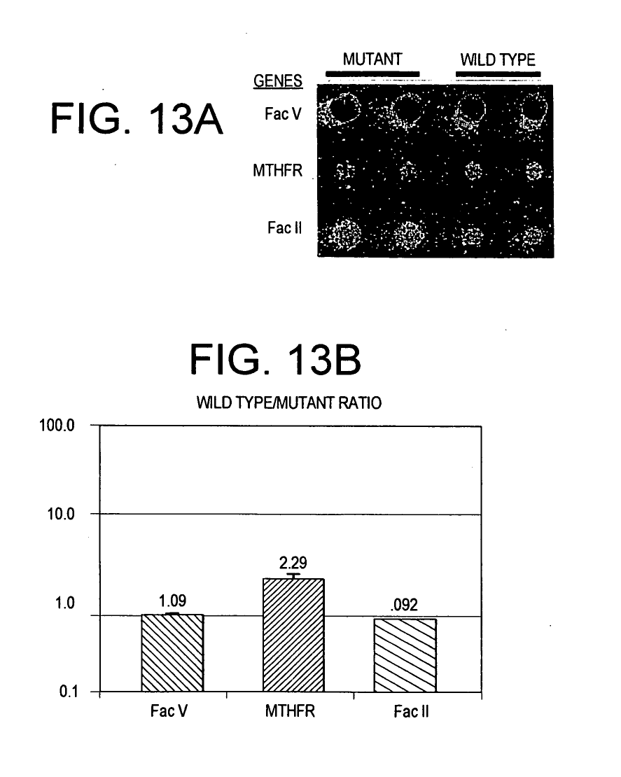

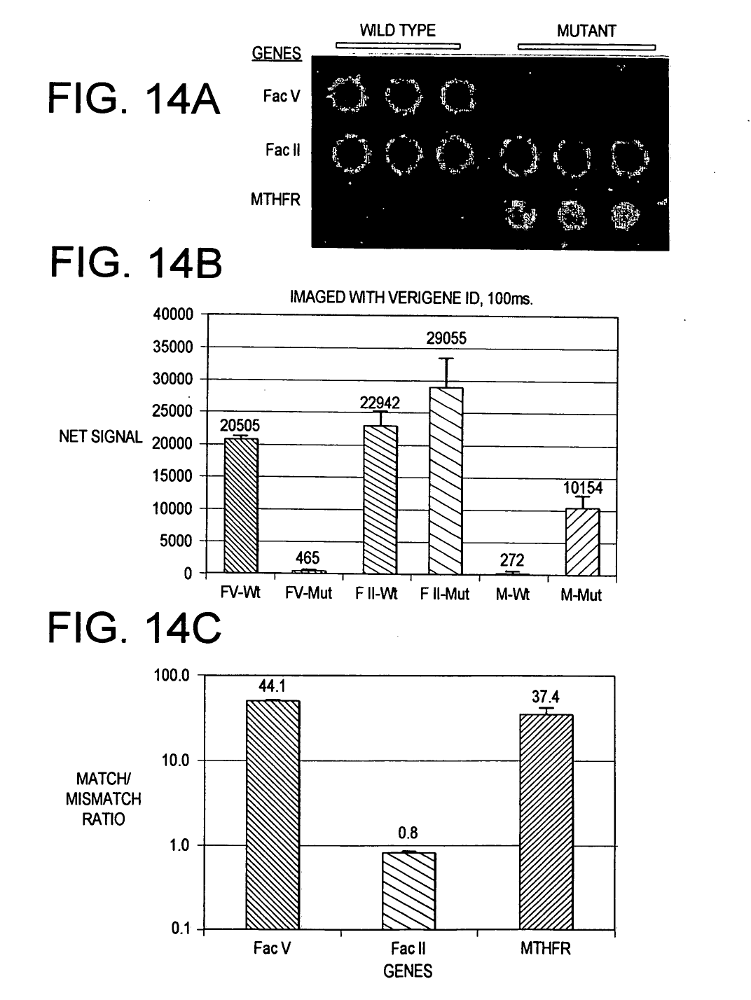

The reproducibility of the two-step hybridization method was examined by performing 10 identical hybridization with 5 μg of wild type whole genomic DNA in separate wells on a single slide. After accounting for the standard deviation, the net signal intensities for the match and mismatch in the 10 separate hybridization wells as shown in FIG. 11 did not overlap, indicating that for each hybridization reaction, the SNP configuration of the input DNA could be reliably determined. Next, the method was used to detect Factor V, Factor II, and MTHFR SNPs and wild type genes in the same sample preparation. Capture oligonucleotides for factor V, factor II, or MTHFR were incubated with 5 μg of whole genomic DNA under hybridization conditions described above. Nanoparticle probes specific for detecting factor V, factor II, or MTHFR were added in the second step. The results of this experiment, shown in FIGS. 12-14, showed that the SNP configuration of at least three different genes could be analyzed simultaneously in a single array, under the same conditions. FIG. 13 shows the results of this multiplex SNP detection in a patient DNA sample (GM16028) that was heterozygous for each gene. FIG. 14 shows the results of the multiplex SNP detection in a patient who was heterozygous for factor II, wild type for factor V, and mutant for MTHFR. The method accurately identified the genotype of the patient (patient sample GM00037). These results showed that the discrimination power was sufficiently strong to discriminate between a homozygous and heterozygous mutant gene. For instance, a person can be homozygous wt, mutant or heterozygous (meaning one wt and one mutant gene) for any given SNP. These three different conditions could be correctly identified for three separate SNP sites independently, in a single assay. The results demonstrated that the methods of the invention could simultaneously identify multiple SNPs in a single sample. While only three SNPs were examined in the experiment, one of skill in the art will recognize that this is only a representative number. Many more SNP sites could be tested within the same array.

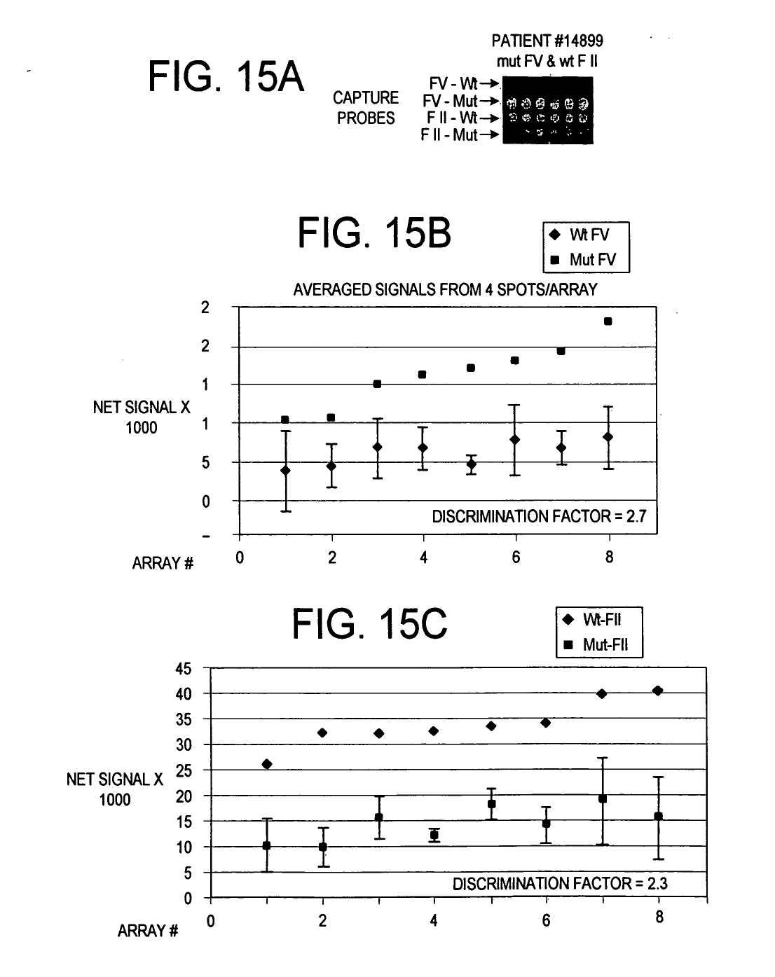

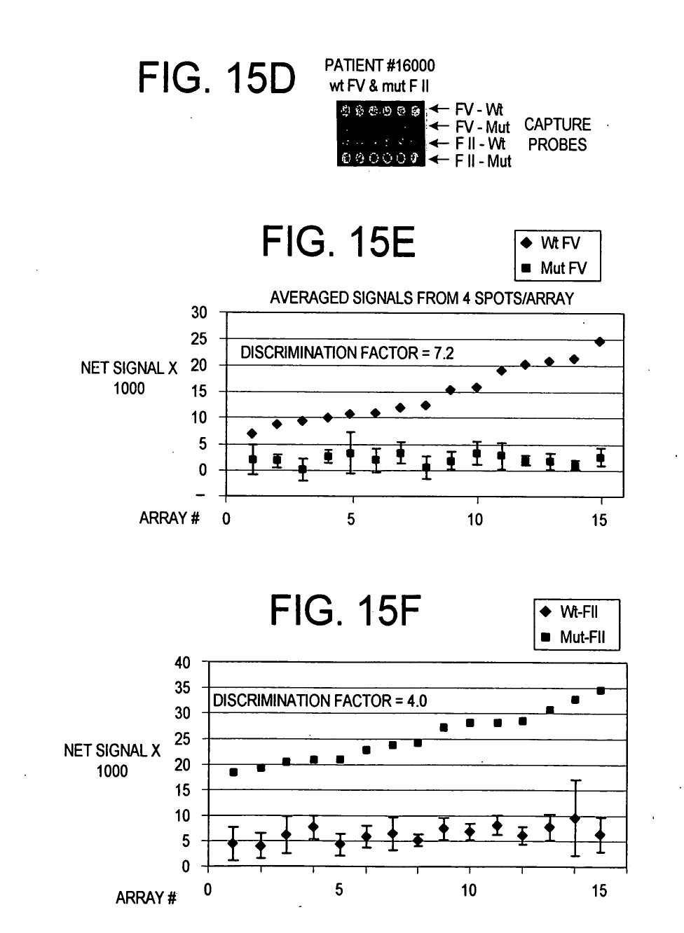

In addition to these experiments, two different investigators separately hybridized eight different slides with the DNA from 2 different patients (1 array per slide for patient GM14899 DNA and 2 arrays/slide for patient GM1600 DNA) using the methods described in these Examples. Each array had 4 repeat spots for each of 2 genes (factor II and factor V) and for each type of capture probe (mutant or wt). The net signal intensities were averaged, sorted, and then plotted starting with the lowest signal intensity. For the mismatch signals (the lower ones on each plot) three times the standard deviation was added to the average net signal. The mutant and corresponding wt signal were always plotted above each other. As shown in FIG. 15, even for the smallest signal intensities, the net signal of the match was always larger than the net signal plus three-time standard deviation of the mismatch signal. Thus, in each case the correct SNP configuration could be determined with better than 99% reliability. The results further demonstrate the reproducibility and robustness of the methods described herein.

### Example 2

Hybridization Conditions for Methods of the Invention

Standard recommendations [T. Maniatis, E. F. Fritsch, and J. Sambrook in “Molecular Cloning, A Laboratory Manual”, Cold Spring Harbor Laboratory, 1982, p324) for efficient hybridization reactions described in the art typically stipulate a hybridization temperature that is ˜10-20 degrees centigrade below the Tm that is calculated for the hybridization conditions one has chosen, including salt and formamide concentration. There are different methods to calculate Tm's, each based on the exact oligonucleotide sequence and buffer conditions. For example, such calculations can be made using computer programs, which are commercially available or available online, such as the HYTHER™ server that was developed and is maintained at the Wayne State University web site. Using all available programs on the HYTHER™ server for these calculations, the inventors computed the Tm's for both capture and detection probes (i.e. oligonucleotides). As shown in Table 1, the Tm's for the capture probes are either below or very near the temperature that was chosen for the hybridization (i.e. 40° C.). Thus, very low hybridization efficiency would be expected under these conditions. Moreover, capture oligonucleotides are attached to a substrate surface directly, i.e. without a linker sequence, meaning that the oligonucleotides closest to the surface may not be able to participate in the hybridization to the target sequence, thereby reducing the effective Tm even further. Based on the teachings in the art, the conditions used in the methods of the invention unexpectedly achieved an efficient hybridization, especially in the case where the target sequence represents only a minute fraction (i.e. 1/100,000,000 or a 1 million's %) of the complex DNA mixture that the human genome represents.

TABLE 1

TM calculated with HyTher ™
(Wayne State University)

TM correction
for hybridization
to surface bound
probes according to:

| | | | |
|---|---|---|---|
| | No | Santalucia | Fotin |
| | corrections | et al. | et al. |
| Sequence | (35% FM) | (35% FM) | (35% FM) |

Capture

| | | | | |
|---|---|---|---|---|
| FII- | CTCAGCGAGCCTCAATGCTCCC | 46.7 | 37.0 | 45.0 |
| SNP/Cap1- | | | | |
| wt22 (SEQ | | | | |
| ID NO:5) | | | | |
| FII- | CTCTCAGCAAGCCTCAATGCTCC | 47.2 | 35.7 | 46.3 |
| SNP/Cap1- | | | | |
| mut23 | | | | |
| MTHFR- | GATGAAATCGGCTCCCGCAGAC | 40.3 | 35.5 | 43.0 |
| SNP/Cap6- | | | | |
| wt22 (SEQ | | | | |
| ID NO:6) | | | | |
| MTHFR- | ATGAAATCGACTCCCGCAGACA | 40.7 | 36.2 | 44.0 |
| SNP/Cap7- | | | | |
| mut22 (SEQ | | | | |
| ID NO:2) | | | | |
| FV-Cap-WT- | TGGACAGGCGAGGAATACAGGTAT | 44.8 | 35.5 | 42.9 |
| 24 (SEQ ID | | | | |
| NO:15) | | | | |
| FV-Cap- | CTGGACAGGCAAGGAATACAGGTATT | 44.5 | 35.8 | 42.9 |
| mut26 (SEQ | | | | |
| ID NO:16) | | | | |
| Probe | | | | |
| FV-46 (SEQ | 5′ Epi-CCA CAG AAA ATG ATG CCC AGT GCT | 54.8 | 49.2 | 57.6 |
| ID NO:12) | TAA CAA GAC CAT ACT ACA GTG A 3′ | | | |
| FII-ProI-47 | 5′ Epi-TCC TGG AAC CAA TCC CGT GAA AGA | 52.2 | 46.9 | 54.8 |
| (SEQ ID | ATT ATT TTT GTG TTT CTA AAA CT 3′ | | | |
| NO:10) | | | | |
| MTHFR-Pro | 5′ Epi-AAA GAT CCC GGG GAC GAT GGG GCA | 68.4 | 58.6 | 68.5 |
| II-58 (SEQ | AGT GAT GCC CAT GTC GGT GCA TGC CTT CAC | | | |
| ID NO:8) | AAA G 3′ | | | |

### Example 3

Preparation of Nanoparticle-Oligonucleotide Conjugate Probes

In this Example, a representative nanoparticle-oligonucleotide conjugate detection probe was prepared for the use in the PCR amplification of mecA and Tuf gene targets. Gold nanoparticle-oligonucleotide probes to detect for the target mecA or Tuf gene sequences was prepared using procedures described in PCT/US97/12783, filed Jul. 21, 1997; PCT/US00/17507, filed Jun. 26, 2000; PCT/US01/01190, filed Jan. 12, 2001, which are incorporated by reference in their entirety.

(a) Preparation Of Gold Nanoparticles

Gold colloids (13 nm diameter) were prepared by reduction of HAuCl₄ with citrate as described in Frens, 1973, Nature Phys. Sci., 241:20 and Grabar, 1995, Anal. Chem. 67:735. Briefly, all glassware was cleaned in aqua regia (3 parts HCl, 1 part HNO₃), rinsed with Nanopure H₂O, then oven dried prior to use. HAuCl₄ and sodium citrate were purchased from Aldrich Chemical Company. Aqueous HAuCl₄ (1 mM, 500 mL) was brought to reflux while stirring. Then, 38.8 mM sodium citrate (50 mL) was added quickly. The solution color changed from pale yellow to burgundy, and refluxing was continued for 15 min. After cooling to room temperature, the red solution was filtered through a Micron Separations Inc. 1 micron filter. Au colloids were characterized by UV-vis spectroscopy using a Hewlett Packard 8452A diode array spectrophotometer and by Transmission Electron Microscopy (TEM) using a Hitachi 8100 transmission electron microscope. Gold particles with diameters of 13 nm will produce a visible color change when aggregated with target and probe oligonucleotide sequences in the 10-35 nucleotide range.

(b) Synthesis Of Steroid Disulfide

An oligonucleotide complementary to a segment of the mecA and Tuf DNA sequences were synthesized on a 1 micromole scale using a Milligene Expedite DNA synthesizer in single column mode using phosphoramidite chemistry. Eckstein, F. (ed.) Oligonucleotides and Analogues: A Practical Approach (IRL Press, Oxford, 1991). All solutions were purchased from Milligene (DNA synthesis grade). Average coupling efficiency varied from 98 to 99.8%, and the final dimethoxytrityl (DMT) protecting group was not cleaved from the oligonucleotides to aid in purification.

To facilitate hybridization of the probe sequence with the target, a deoxyadenosine oligonucleotide (da₁₅ peg for all probes except probe Tuf 2 which has a da₁₀ peg) was included on the 5′ end in the probe sequence as a spacer.

To generate 5′-terminal steroid-cyclic disulfide oligonucleotide derivatives (see Letsinger et al., 2000, Bioconjugate Chem. 11:289-291 and PCT/US01/01190 (Nanosphere, Inc.), the disclosure of which is incorporated by reference in its entirety), the final coupling reaction was carried out with a cyclic dithiane linked epiandrosterone phosphoramidite on Applied Biosystems automated synthesizer, a reagent that prepared using 1,2-dithiane-4,5-diol, epiandrosterone and p-toluenesulphonic acid (PTSA) in presence of toluene. The phosphoramidite reagent may be prepared as follows: a solution of epiandrosterone (0.5 g), 1,2-dithiane-4,5-diol (0.28 g), and p-toluenesulfonic acid (15 mg) in toluene (30 mL) was refluxed for 7 h under conditions for removal of water (Dean Stark apparatus); then the toluene was removed under reduced pressure and the reside taken up in ethyl acetate. This solution was washed with water, dried over sodium sulfate, and concentrated to a syrupy reside, which on standing overnight in pentane/ether afforded a steroid-dithioketal compound as a white solid (400 mg); Rf (TLC, silica plate, ether as eluent) 0.5; for comparison, Rf values for epiandrosterone and 1,2-dithiane-4,5-diol obtained under the same conditions are 0.4, and 0.3, respectively. Recrystallization from pentane/ether afforded a white powder, mp 110-112° C.; ¹H NMR, δ 3.6 (1H, C³OH), 3.54-3.39 (2H, m 20CH of the dithiane ring), 3.2-3.0 (4H, m 2CH₂S), 2.1-0.7 (29H, m steroid H); mass spectrum (ES⁺) calcd for C₂₃H₃₆O₃S₂ (M+H) 425.2179, found 425.2151. Anal. (C₂₃H₃₇O₃S₂) S: calcd, 15.12; found, 15.26. To prepare the steroid-disulfide ketal phosphoramidite derivative, the steroid-dithioketal (100 mg) was dissolved in THF (3 mL) and cooled in a dry ice alcohol bath. N,N-diisopropylethylamine (80 μL) and β-cyanoethyl chlorodiisopropylphosphoramidite (80 μL) were added successively; then the mixture was warmed to room temperature, stirred for 2 h, mixed with ethyl acetate (100 mL), washed with 5% aq. NaHCO₃ and with water, dried over sodium sulfate, and concentrated to dryness. The residue was taken up in the minimum amount of dichloromethane, precipitated at −70° C. by addition of hexane, and dried under vacuum; yield 100 mg; ³¹P NMR 146.02. The epiandrosterone-disulfide linked oligonucleotides were synthesized on Applied Biosystems automated gene synthesizer without final DMT removal. After completion, epiandrosterone-disulfide linked oligonucleotides were deprotected from the support under aqueous ammonia conditions and purified on HPLC using reverse phase column.

Reverse phase HPLC was performed with a Dionex DX500 system equipped with a Hewlett Packard ODS hypersil column (4.6×200 mm, 5 mm particle size) using 0.03 M Et₃NH⁺ OAc⁻-buffer (TEAA), pH 7, with a 1%/min. gradient of 95% CH₃CN/5% TEAA. The flow rate was 1 mL/min. with UV detection at 260 nm. Preparative HPLC was used to purify the DMT-protected unmodified oligonucleotides. After collection and evaporation of the buffer, the DMT was cleaved from the oligonucleotides by treatment with 80% acetic acid for 30 min at room temperature. The solution was then evaporated to near dryness, water was added, and the cleaved DMT was extracted from the aqueous oligonucleotide solution using ethyl acetate. The amount of oligonucleotide was determined by absorbance at 260 nm, and final purity assessed by reverse phase HPLC.

(c) Microarray Preparation

3′-amino and 5′-amino containing DNA was synthesized by following standard protocol for DNA synthesis on DNA synthesizer. The amine modified DNA was attached to the aldehyde microarray slide by printing a 1 mM DNA solution in ArrayIt buffer plus (Catalog no. MSP, Company nameTelechem, citySunnyvale, StateCA). An Affymetrix® GMS 417 arrayer (Affymetrix, city Santa Clara, state CA) with 500 micron printing pins was used to orient the microarray on the slide. The microarray slide was purchased from Telechem (catalog no. SMM, city Sunnyvale, state CA) with an aldehyde functionalized surface. After printing, the slides were placed in a humidified chamber at ambient temperature for 12-18 hrs. The slides were removed and dried under vacuum for 30 min to 2 hrs. The slides were then subjected to two washes in 0.2% w/v SDS and two washes in water to remove any remaining unbound DNA. The slides were then treated with a solution of 2.5 M sodium borohydride in 1× PBS with 20% v/v 100% ethanol by soaking for 5 min. The slides were then washed three times with 0.2% w/v SDS and twice with water and centrifuged dry.

(d) Attachment Of Oligonucleotides To Gold Nanoparticles

A colloidal solution of citrate stabilized gold nanoparticles (about 10 nM), prepared as described in part A above, was mixed with sulfur modified-a_{15 peg}-probe oligonucleotide (4 μM), prepared as described in part B, and allowed to stand for 6 hours at room temperature in 20 ml scintillation vials. 0.1 M sodium hydrogen phosphate buffer, pH 7.0, and of 5.0 M NaCl were each added to the solution in amounts resulting in a solution at 0.01 M sodium hydrogen phosphate and 0.1 M NaCl and allowed to stand for an additional 16 hours. Sodium chloride was added in a gradient over 36 hrs to 0.8 M NaCl and the resulting solution was incubated for an additional 18 hours. The solution was aliquoted into 1 ml eppendorf tubes and centrifuged at 14,000 rpm in an Eppendorf Centrifuge 5414 for 25 minutes to give a very pale pink supernatant containing most of the oligonucleotide (as indicated by the absorbance at 260 nm) along with 7-10% of the colloidal gold (as indicated by the absorbance at 520 nm), and a compact, dark, gelatinous residue at the bottom of the tube. The supernatant was removed, and the residue was resuspended in the desired aqueous buffer. In this Example, the buffer used includes 0.1M NaCl, 10 mM sodium citrate, and 0.01% sodium azide at pH 7.

The following nanoparticle-oligonucleotide detection probes and amine modified DNA capture probes specific for mecA or Tuf DNA were prepared in this manner: Here, the oligonucleotide probe can be modified with an amine and immobilized on the glass slide as a capture probe or modified with an epiandrosterone linker and immobilized on the gold particle as a detection probe. In other words, the oligonucleotides and its reverse complements can be interchangeably used as either capture probes or nanoparticle detection probes.

(a)

Detection Probes
Probe Tuf 1:

(SEQ ID NO:17)

gold-S′-5′-[a₁₅PEG-ttctatttccgtactactgac-3′]n
Probe Tuf 2:

(SEQ ID NO:18)

gold-S′-5′-[a₁₅peg-ttctatttccgtactactgacgtaa
ct-3′]n
Probe Tuf 3:

(SEQ ID NO:19)

5′-[amine-peg₃-ccattcttctcaaactatcgt-3′]
Probe Tuf 4:

(SEQ ID NO:20)

5′-[amine-peg₃-ccattcttcactaactatcgc-3′]
Probe Tuf 5:

(SEQ ID NO:21)

5′-[amine-peg₃-cacactccattcttctcaaact-3′]
Probe Tuf 6:

(SEQ ID NO:22)

5′-[amine-peg₃-cacactccattcttcactaact-3′]
Probe Tuf 7:

(SEQ ID NO:23)

5′-[amine-peg₃-atatgacttcccaggtgac-3′]
Probe Tuf 8:

(SEQ ID NO:24)

5′-[amine-peg₃-gtagatacttacattcca-3′]
Probe Tuf 9:

(SEQ ID NO:25)

5′-[amine-peg₃-gttgatgattacattcca-3′]
Probe Tuf 10:

(SEQ ID NO:26)

5′-[amine-peg₃-ccattcttcactaactaccgc-3′]
Probe Tuf 11:

(SEQ ID NO:27)

5′-[amine-peg₃-catacgccattcttcactaact-3′]
Probe Tuf 15:

(SEQ ID NO:28)

5′-[amine-peg₃-ccattcttctctaactatcgt-3′]
Probe Tuf 16:

(SEQ ID NO:29)

5′-[amine-peg₃-ccattcttcacaaactatcgt-3′]
Probe Tuf 17:

(SEQ ID NO:30)

5′-[amine-peg₃-ccattcttcagtaactatcgc-3′]
Probe Tuf 18:

(SEQ ID NO:31)

5′-[amine-peg₃-ccattcttcagtaactaccgc-3′]
Probe Tuf 19:

(SEQ ID NO:32)

5′-[amine-peg₃-ccattcttctcaaactaccgc-3′]
Probe Tuf 20:

(SEQ ID NO:33)

5′-[amine-peg₃-ccattcttctctaactaccgt-3′]
Probe Tuf 21:

(SEQ ID NO:34)

5′-[amine-peg₃-catacgccattcttcagtaact-3′]
Probe Tuf 22:

(SEQ ID NO:35)

5′-[amine-peg₃-cacactccattcttcagtaact-3′]
Probe Tuf 23:

(SEQ ID NO:36)

5′-[amine-peg₃-catactccattcttcactaact-3′]
Probe Tuf 24:

(SEQ ID NO:37)

5′-[amine-peg₃-catacaccattcttctcaaact-3′]
Probe Tuf 25:

(SEQ ID NO:38)

5′-[amine-peg₃-catactccattcttctctaact-3′]
Probe Tuf 26:

(SEQ ID NO:39)

5′-[amine-peg₃-cacactccattcttcacaaact-3′]
Probe Tuf 27:

(SEQ ID NO:40)

5′-[amine-peg₃-cacactccattcttctctaact-3′]
Probe mecA 1:

(SEQ ID NO:41)

5′-[amine-peg₃-tcgatggtaaaggttggc-3′]
Probe mecA 2:

(SEQ ID NO:42)

5′-[amine-peg₃-atggcatgagtaacgaagaatata-3′]
Probe mecA 3:

(SEQ ID NO:43)

gold-S′-5′-[amine-peg₃-aaagaacctctgctcaacaag-3′]ₙ
Probe mecA 4:

(SEQ ID NO:44)

gold-S′-5′-[amine-peg₃-gcacttgtaagcacaccttcat-3′]ₙ
Probe mecA 6:

(SEQ ID NO:45)

5′-[amine-peg₃-ttccagattacaacttcacca-3′]
Probe 16S 12:

(SEQ ID NO:46)

5′-[amine-peg₃-gttcctccatatctctgcg-3′]
Probe 16S 13:

(SEQ ID NO:47)

gold-S′-5′-[amine-peg₃-atttcaccgctacacatg-3′]ₙ

S′ indicates a connecting unit prepared via an epiandrosterone disulfide group; n represents a variable number of oligonucleotides were used in preparing the nanoparticle-oligonucleotide conjugates.

TABLE 2

| | SEQ | | |
|---|---|---|---|
| | ID | | |
| Name | NO: | Sequence 5′→3′ | Staph Species |
| Tuf 1 | 17 | TTCTATTTCCGTACTACTGAC | Tuf gene General |
| | 48 | GTCAGTAGTACGGAAATAGAA | |
| | | (reverse complement) | |
| Tuf 2 | 18 | TTCTATTTCCGTACTACTGACGTAACT | Tuf gene General |
| | 49 | AGTTACGTCAGTAGTACGGAAATAGAA | |
| | | (reverse complement) | |
| Tuf 3 | 19 | CCATTGTTCTCAAACTATCGT | S. aureus |
| | 50 | ACGATAGTTTGAGAAGAATGG | |
| | | (reverse complement) | |
| Tuf 4 | 20 | CCATTCTTCACTAACTATCGC | S. epidermidis |
| | 51 | GCGATAGTTAGTGAAGAATGG | |
| | | (reverse complement) | |
| Tuf 5 | 21 | CACACTCCATTCTTCTCAAAGT | S. aureus |
| | 52 | AGTTTGAGAAGAATGGAGTGTG | |
| | | (reverse complement) | |
| Tuf 6 | 22 | CACACTCCATTCTTCACTAACT | S. epidermidis |
| | 53 | AGTTAGTGAAGAATGGAGTGTG | |
| | | (reverse complement) | |
| Tuf 7 | 23 | ATATGACTTCCCAGGTGAC | Tuf gene general |
| | 54 | GTCACCTGGGAAGTCATAT | |
| | | (reverse complement) | |
| Tuf 8 | 24 | GTAGATACTTACATTCCA | S. aureus |
| | 55 | TGGAATGTAAGTATCTAC | |
| | | (reverse complement) | |
| Tuf 9 | 25 | GTTGATGATTACATTCCA | S. epidermidis |
| | 56 | TGGAATGTAATCATCAAC | |
| | | (reverse complement) | |
| Tuf 10 | 26 | CCATTCTTCACTAACTACGGC | S. saprophyticus |
| | 57 | GCGGTAGTTAGTGAAGAATGG | S. simulans |
| | | (reverse complement) | |
| Tuf 11 | 27 | CATACGCCATTGTTCACTAACT | S. saprophyticus |
| | 58 | AGTTAGTGAAGAATGGCGTATG | |
| | | (reverse complement) | |
| Tuf 15 | 28 | CCATTCTTCTCTAACTATCGT | S. hominis |
| | 59 | ACGATAGTTAGAGAAGAATGG | |
| | | (reverse complement) | |
| Tuf 16 | 29 | CCATTCTTCACAAACTATCGT | S. haemoylticus |
| | 60 | ACGATAGTTTGTGAAGAATGG | |
| | | (reverse complement) | |
| Tuf 17 | 30 | CCATTCTTCAGTAACTATCGC | S. cohnii |
| | 61 | GCGATAGTTACTGAAGAATGG | |
| | | (reverse complement) | |
| Tuf 18 | 31 | CCATTCTTCAGTAACTACCGC | S. warneri |
| | 62 | GCGGTAGTTACTGAAGAATGG | S. capitis |
| | | (reverse complement) | |
| Tuf 19 | 32 | CCATTCTTCTCAAACTACCGC | S. lugdunenis |
| | 63 | GCGGTAGTTTGAGAAGAATGG | |
| | | (reverse complement) | |
| Tuf 20 | 33 | CCATTCTTCTCTAACTACCGT | S. auricularis |
| | 64 | ACGGTAGTTAGAGAAGAATGG | |
| | | (reverse complement) | |
| Tuf 21 | 34 | CATACGCCATTCTTCAGTAACT | S. cohnii |
| | 65 | AGTTACTGAAGAATGGCGTATG | |
| | | (reverse complement) | |
| Tuf 22 | 35 | CACACTCCATTCTTCAGTAACT | S. warneri |
| | 66 | AGTTACTGAAGAATGGAGTGTG | S. capitis |
| | | (reverse complement) | |
| Tuf 23 | 36 | CATACTCCATTCTTCACTAACT | S. simulans |
| | 67 | AGTTAGTGAAGAATGGAGTATG | |
| | | (reverse complement) | |
| Tuf 24 | 37 | CATACACCATTCTTCTCAAACT | S. lugdunensis |
| | 68 | AGTTTGAGAAGAATGGTGTATG | |
| | | (reverse complement) | |
| Tuf 25 | 38 | CATACTCCATTCTTCTCTAACT | S. hominis |
| | 69 | AGTTAGAGAAGAATGGAGTATG | |
| | | (reverse complement) | |
| Tuf 26 | 39 | CACACTCCATTCTTCACAAACT | S. haemolyticus |
| | 70 | AGTTTGTGAAGAATGGAGTGTG | |
| | | (reverse complement) | |
| Tuf 27 | 40 | CACACTCCATTCTTCTCTAACT | S. auricularis |
| | 71 | AGTTAGAGAAGAATGGAGTGTG | |
| | | (reverse complement) | |
| mecA 1 | 41 | TCGATGGTAAAGGTTGGC | mecA gene |
| | 72 | GCCAACCTTTACCATCGA | |
| | | (reverse complement) | |
| mecA 2 | 42 | ATGGCATGAGTAACGAAGAATATA | mecA gene |
| | 73 | TATTGTATTCGTTACTCATGCCAT | |
| | | (reverse complement) | |
| mecA 3 | 43 | AAAGAACCTCTGCTCAACAAG | mecA gene |
| | 74 | CTTGTTGAGCAGAGGTTCTTT | |
| | | (reverse complement) | |
| mecA 4 | 44 | GCACTTGTAAGCACACGTTCAT | mecA gene |
| | 75 | ATGAAGGTGTGCTTACAAGTGC | |
| | | (reverse complement) | |
| mecA 6 | 45 | TTCCAGATTACAACTTCACCA | |
| | 76 | TGGTGAAGTTGTAATCTGGAA | |
| | | (reverse complement) | |
| 16S 12 | 46 | GTTCCTCCATATCTCTGCG | 16S rRNA |
| | 77 | CGCAGAGATATGGAGGAAC | |
| | | (reverse complement) | |
| 16S 13 | 47 | ATTTCACCGCTACACATG | 16S rRNA |
| | 78 | CATGTGTAGCGGTGAAAT | |
| | | (reverse complement) | |

### Example 4

### Detection of mecA Gene Sequences from Bacterial Genomic DNA with Gold Nanoparticle Probes

In this Example, a method for detecting mecA gene sequences using gold nanoparticle-based detection in an array format is described. Microarray plates having mecA 2 and mecA 6 oligonucleotides as capture probes were used along with gold nanoparticles labeled with mecA 4 oligonucleotides as a detection probe. The microarray plates, capture probes, and detection probes were prepared as described in Example 3.

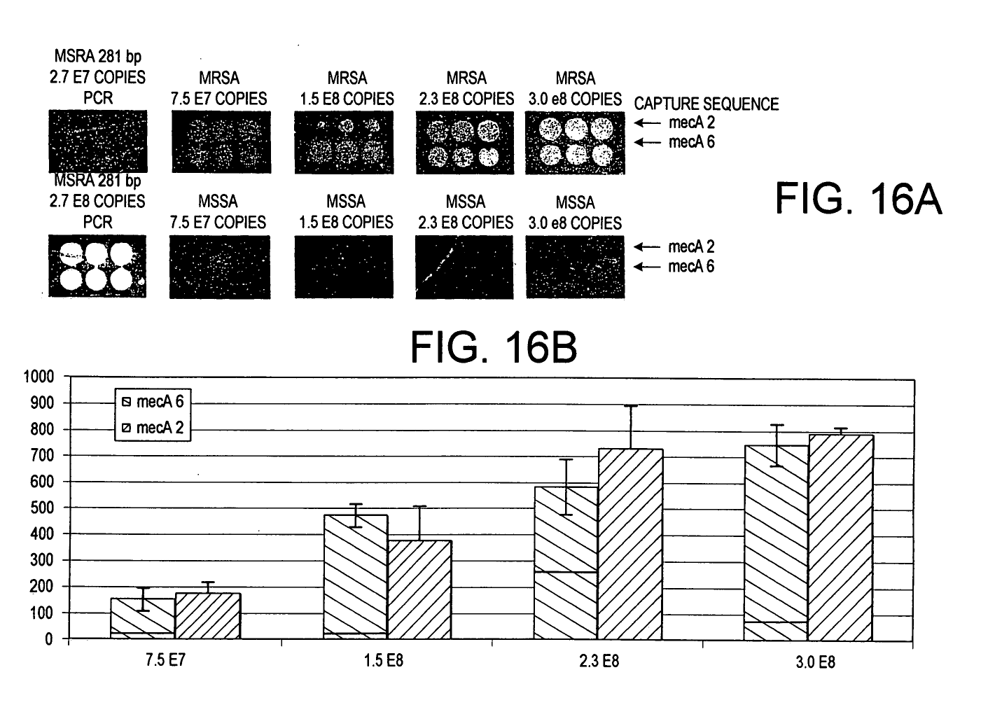

Gold nanoparticles (13 nm diameter) having oligonucleotide probes attached to them prepared as described in Example 3 were used to indicate the presence of DNA from the mecA gene hybridized to a transparent substrate in a three-component sandwich assay format. Nanoparticles having probe oligonucleotides attached to them and genomic DNA targets isolated from methicillin resistant (MecA+) or methicillin sensitive (MecA−) S. aureus bacterial cells were then cohybridized to these substrates. Therefore, the presence of nanoparticles at the surface indicated the detection of the mecA gene sequence, FIG. 16. At the target amounts tested (250 ng (7.5 E7copies)—1 ug (3.0 E8)), the attached nanoparticles could not be visualized with the naked eye. In order to facilitate the visualization of nanoparticles hybridized to the substrate surface, a signal amplification method in which silver ions are catalytically reduced by hydroquinone to form silver metal on the slide surface was employed. Although this method has been used for enlargement of protein- and antibody-conjugated gold nanoparticles in histochemical microscopy studies (Hacker, in Colloidal Gold: Principles, Methods, and Applications, M. A. Hayat, Ed. (Academic Press, San Diego, 1989), vol. 1, chap. 10; Zehbe et al., Am. J. Pathol. 150, 1553 (1997)) its use in quantitative DNA hybridization assays is novel (Tomlinson et al., Anal. Biochem., 171:217 (1988)). Not only did this method allow very low surface coverages of nanoparticle probes to be visualized by a simple flatbed scanner or the naked eye, it also permitted quantification of target hybridization based on the light scattered from the silver amplified gold probes on the stained area. Significantly, the signal intensities obtained from the samples containing methicillin resistant S. aureus genomic DNA were much larger than the signal intensities obtained from methicillin sensitive S. aureus genomic DNA at each genomic DNA amount tested. This demonstrated that this detection methodology can be used for specific detection of the mecA gene in the presence of complex bacterial genomic DNA background, FIG. 16. This result is an extraordinary feature of the nanoparticle-oligonucleotide conjugates which enables ultra-sensitive and -selective detection of nucleic acids. It also should be noted that this procedure requires no enzymatic target or signal amplification procedures, providing a novel method of gene detection from bacterial genomic DNA samples.

(a) Target DNA Preparation

Purified total genomic DNA isolated from Staphylococcus bacterial cells was purchased from ATCC. The total genomic DNA was fragmented by sonication to shear DNA molecules as described in Example 5 (see below) prior to hybridization on the array.

(b) MecA Gene Detection Assay

(ii) Assay Procedure

Reaction mixtures of bacterial genomic DNA ranging in amount from 250 ng-1 ug and 1 nM nanoparticle probes were made in 1× hybridization buffer (5×SSC, 0.05% Tween 20). The reaction mixture was heated to 95° C. for 5 minutes. Subsequently, 10-25 ul of the reaction mixture was added to the microarray surface and hybridized at 40° C. and 90% relative humidity for 2 hours. The microarray surface was washed for 30 sec in 5×SSC, 0.05% Tween 20 at room temperature, then washed for another 30 sec with 0.5 M NaNO₃ also at room temperature. The microarray was dried and exposed with silver development using commercial grade silver enhancer solutions (Silver Enhancer Kit, Catalog No. SE-100, Sigma, St. Louis) for 4 minutes. The silver stained microarray plate was then washed, dried and imaged using an Arrayworx® scanner (Model No. AWE, Applied Precision, Inc., Issaquah, Wash.).

### Example 5

### Staphylococcal Speciation using Bacterial Genomic DNA and Gold Nanoparticle-Labeled Tuf Probes

In this Example, Staphylococcal speciation was performed via discrimination of Tuf gene sequences corresponding to the species of S. aureus and S. epidermidis. Tuf 372 bp PCR amplicons amplified from total genomic DNA isolated from S. aureus and S. epidermidis bacterial cells served as a positive control to demonstrate sequence specificity of the array. In separate hybridization reactions, total genomic DNA isolated from S. aureus and S. epidermidis bacterial cells was fragmented and hybridized to the micro array plates. Microarray plates included either Tuf 3 and Tuf 4 or Tuf 5 and Tuf 6 capture probes bound thereto. Gold nanoparticles labeled with Tuf 2 oligonucleotides were used as detection probes. The microarray plates, capture and detection probes were prepared as described in Example 3. The Tuf 372 bp amplicon was prepared by conventional PCR amplification procedures.

(a) Target DNA Preparation

The genomic DNA was prepared as follows: genomic DNA isolated from cultured Staphylococcus bacterial cells was purchased from ATCC (American Type Culture Collection). This dry DNA, in >10 ug portions was rehydrated in DNase free water at a volume of 200 ul. This was then sonicated using a Misonix, Ultrasonic cell disruptor XL Farmingdale, N.Y. with 12, ˜0.5 sec pulses at 2 Watts. The total DNA concentration was determined using a commercially available Picogreen kit from Molecular Probes and read on a Tecan spectrafluor plus fluorescence plate reader. The size of the DNA fragments were measured to average 1.5 Kb by performing a smear analysis on an Agilent 2100 Bioanalyzer. The positive control tuf gene 372 base-pair PCR amplicon was prepared from S. aureus or S. epidermidis genomic DNA using conventional PCR amplification techniques.

(b) Tufgene Detection Assay Procedure

In separate hybridization wells, fragmented total genomic DNA isolated from Staphylococcus epidermidis or Staphylococcus aureus bacterial cells (8.0 E07 copies, ˜250 ng) and 1 nM nanoparticle probes were mixed in 1× hybridization buffer (5×SSC, 0.05% Tween 20). As a positive control, PCR-amplifed Tuf gene fragments of the same genomic DNA samples were mixed with probes and buffer in separate hybridization wells on the glass slide. The reaction mixture was heated to 95° C. for 5 minutes. Subsequently, 50 ul of the reaction mixture was added to the microarray surface and hybridized at 45° C. and 90% relative humidity for 1.5 hours. The microarray surface was washed for 30 sec in 0.5 M NaNO₃ at room temperature. The microarray was dried and exposed with silver development using commercial grade silver enhancer solutions (Silver Enhancer Kit, Catalog No. SE-100, Sigma, St. Louis, Mo.) for 4 minutes. The silver stained microarray plate was then washed, dried, and the light scattered from silver amplified nanoparticle probes on the array was imaged and quantified using an Arrayworx® scanner (Model No. AWE, Applied Precision, Issaquah, Wash.).

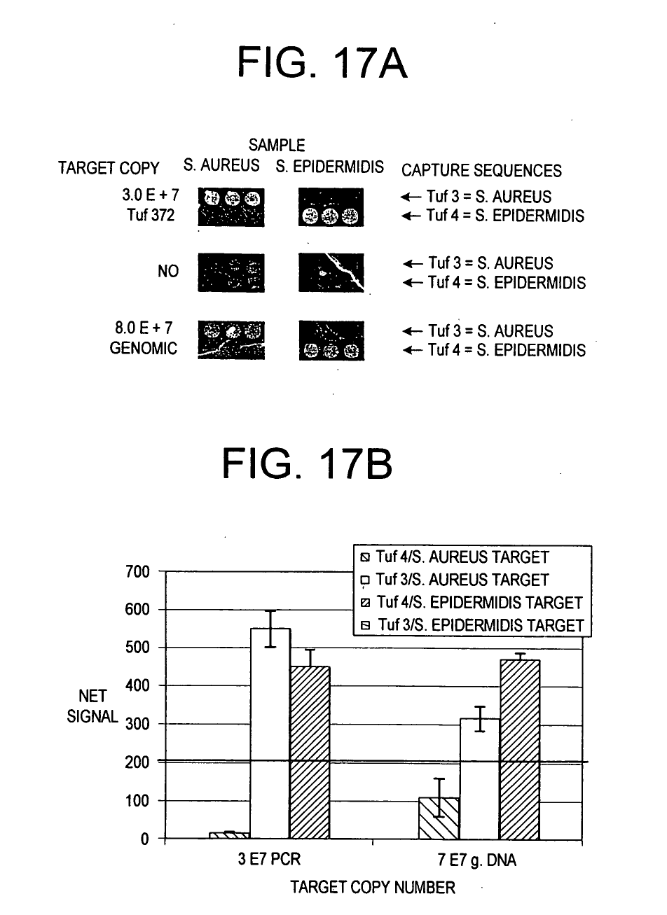

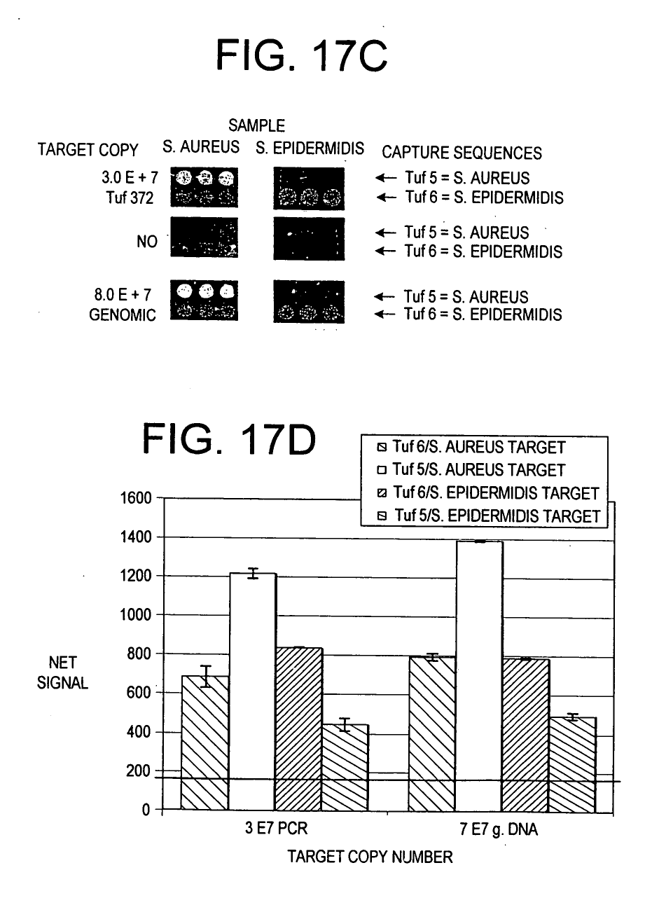

The results are shown in FIGS. 17(a) and (b) for Tuf3 and Tuf4 capture probes, and in FIGS. 17(c) and (d) for Tuf5 and Tuf6 capture probes. Using the Tuf3 and Tuf4 capture probe set, specific signals are observed on the array corresponding to the Staphlococcal species S. aureus and S. epidermidis when genomic DNA is hybridized to the array. This effectively demonstrates that complexity reduction and amplification of tuf gene target by PCR is not required for differentiation of these closely related sequences in the presence of total genomic DNA. Using the Tuf5 and Tuf6 capture probe set, signals corresponding to the appropriate species are also observed, but there is some cross reactivity with the mismatched capture sequence which leads to a lower discrimination ratio. This demonstrates that sequence design is crucial to the accurate identification of species.

### Example 6

Staphylococcal Speciation and Methicillin Resistance Assay using PCR Amplicons and Gold Nanoparticles labeled mecA and Tuf Oligonucleotides as Detection Probes

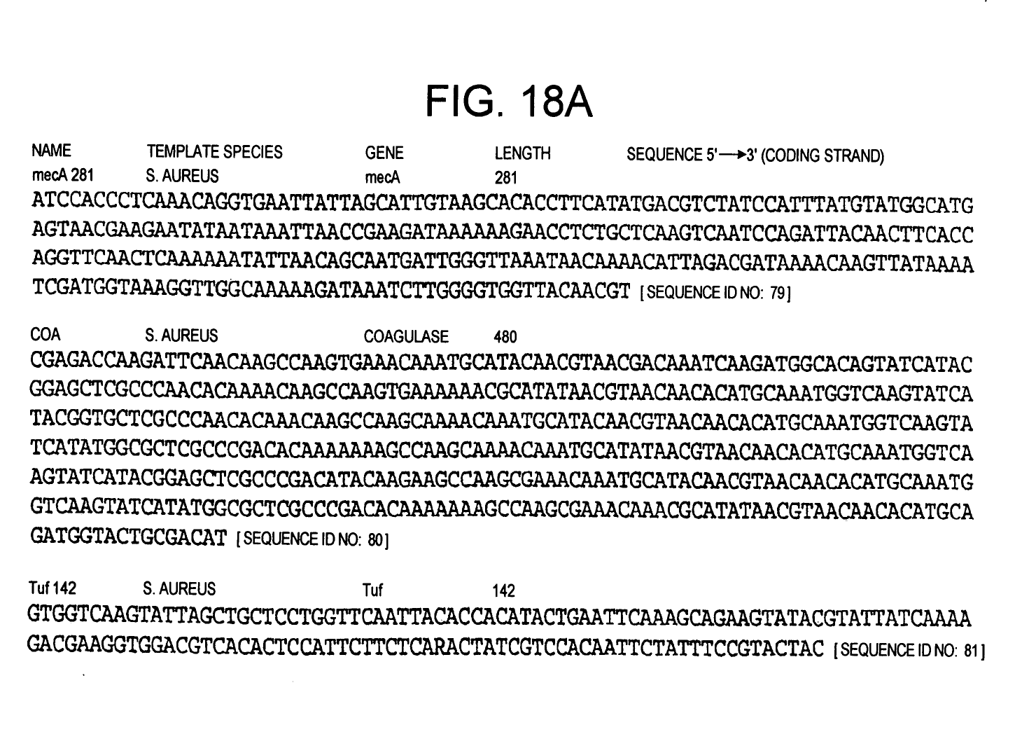

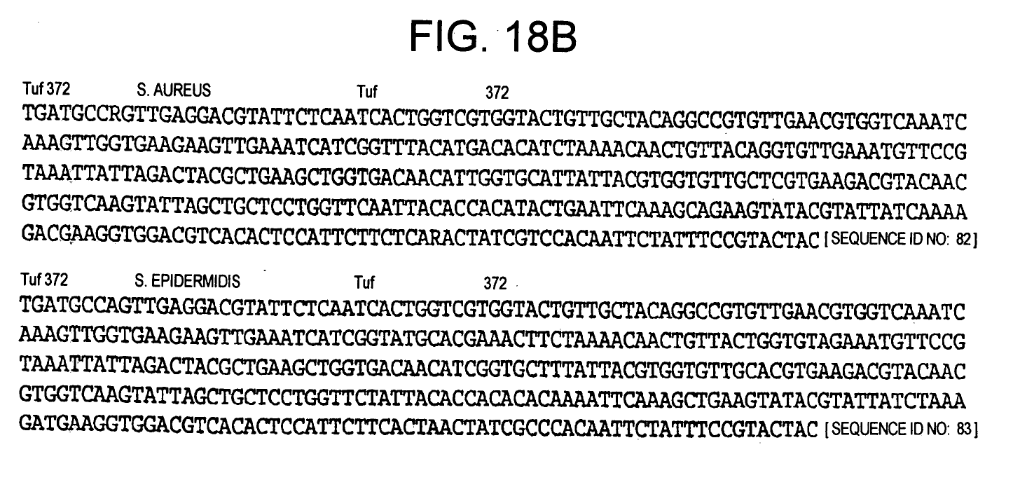

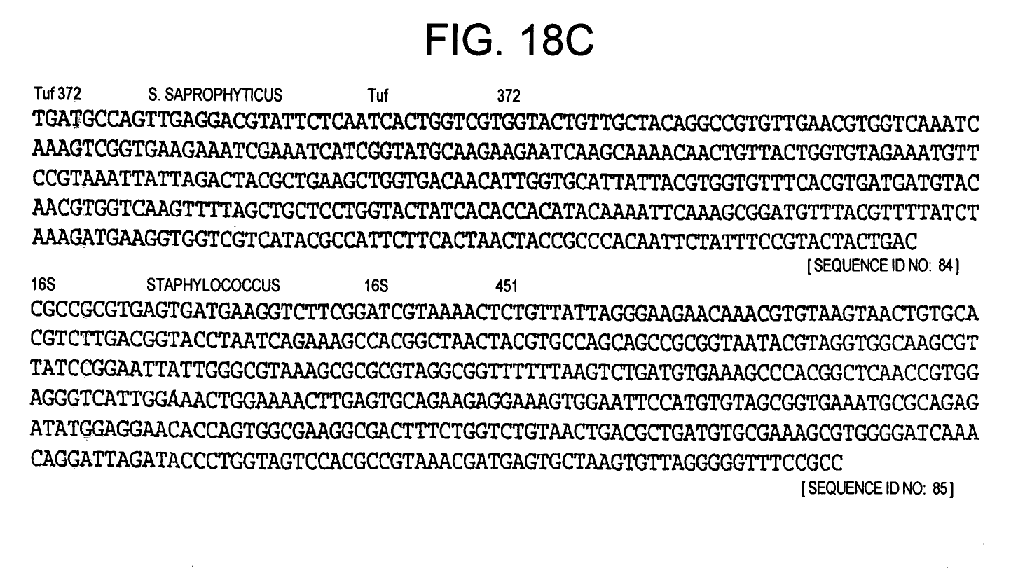

In this Example, an array designed to identify Staphylococcus genus, species, and antibiotic resistance status was fabricated using sequences from the 16S rRNA gene (genus), Tuf gene (species specific captures for S. aureus, S. epidermidis, and S. saprophyticus) and mecA gene (antibiotic resistance status). Note that the S. epidermidis and S. saprophyticus capture probes differ by only a single nucleotide, while the S. aureus and S. epidermidis capture probes differ by three nucleotides. Microarray plates included all of the following sequences: 16S 12, mecA 6, Tuf 3, Tuf 4, Tuf 10 capture probes and one negative hybridization capture probe bound thereto. Gold nanoparticle-labeled Tuf 2, mecA 4, and 16S 12 probes were used as detection probes. The microarray plates, capture and detection probes were prepared as described in Example 4. The specificity of the array was tested using PCR-amplified gene sequences from various methicillin resistant and methicillin sensitive Staphylococcal species (S. aureus, S. epidermidis, and S. saprophyticus). The specific PCR amplified gene fragments used for testing are shown in FIG. 18 (mecA 281, 16S 451, and Tuf 372). The Tuf gene sequences from different Staphylococcus species shown in FIG. 18 were acquired from GenBank.

Target preparation:

The PCR-amplified gene products were prepared using standard PCR amplification procedures.

Assay.

Each reaction consisted of 50 ul of 5×SSC, 0.05% Tween 20, 0.01% BSA, 200 pM each nanoparticle probe, 15% formamide and 750 pM of each target amplicon. The reagents were hybridized for 1 hr at 40 C and 90% humidity. The microarray surface was washed for 30 sec in 0.5 M NaNO₃ at room temperature. The microarray was dried and exposed with silver development using commercial grade silver enhancer solutions (Silver Enhancer Kit, Catalog No. SE-100, Sigma, St. Louis, Mo.) for 4 minutes. The silver stained microarray plate was then washed, dried and imaged using an Arrayworx® scanner (Model No. AWE, Applied Precision, Issaquah, Wash.).

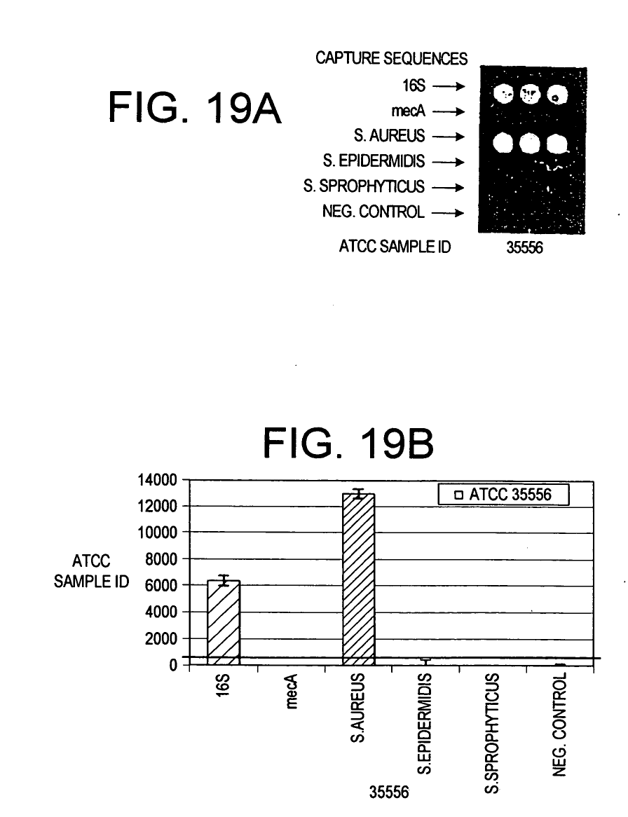

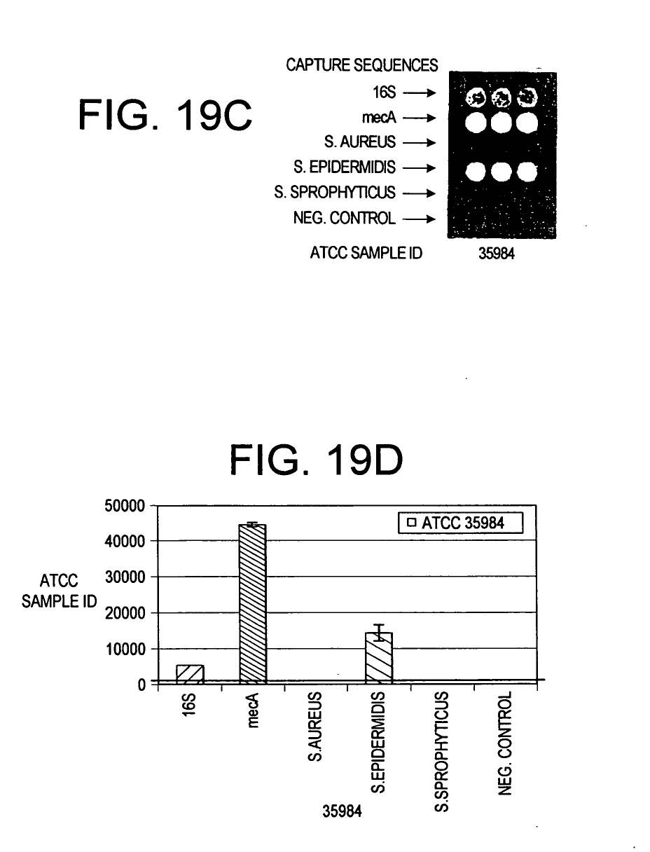

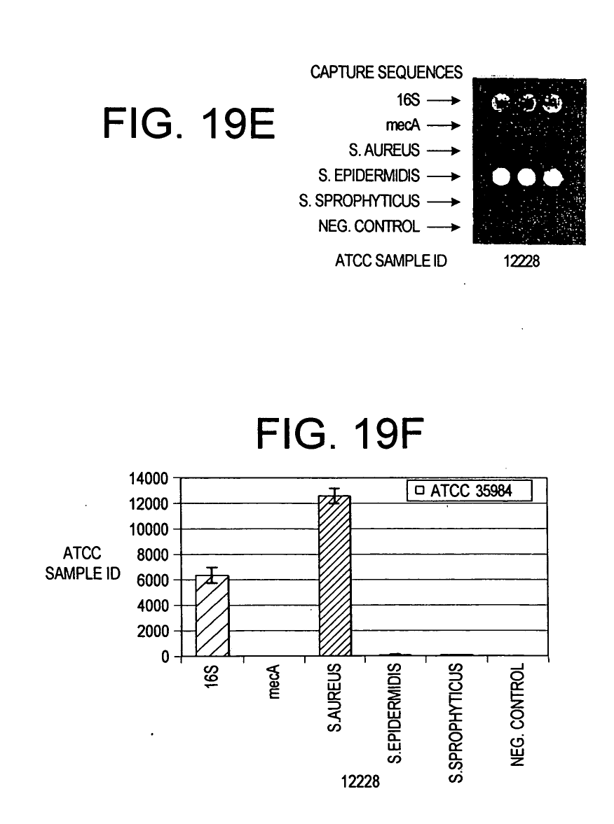

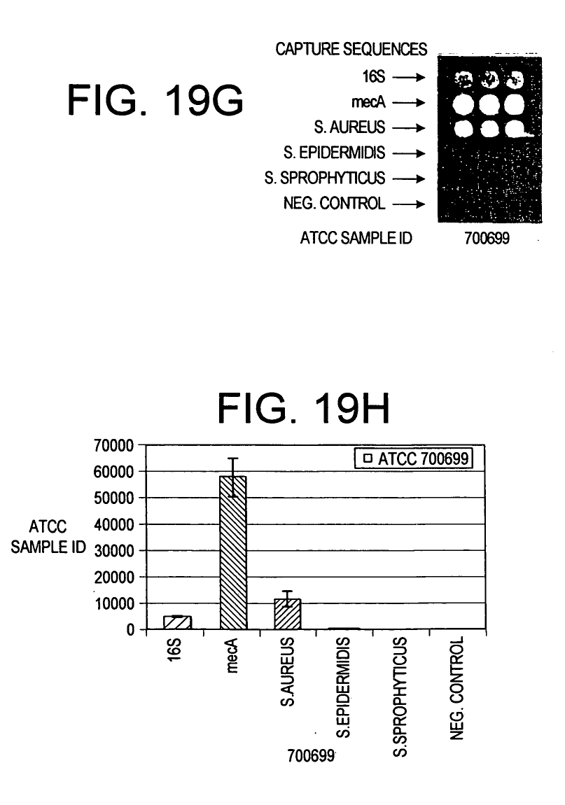

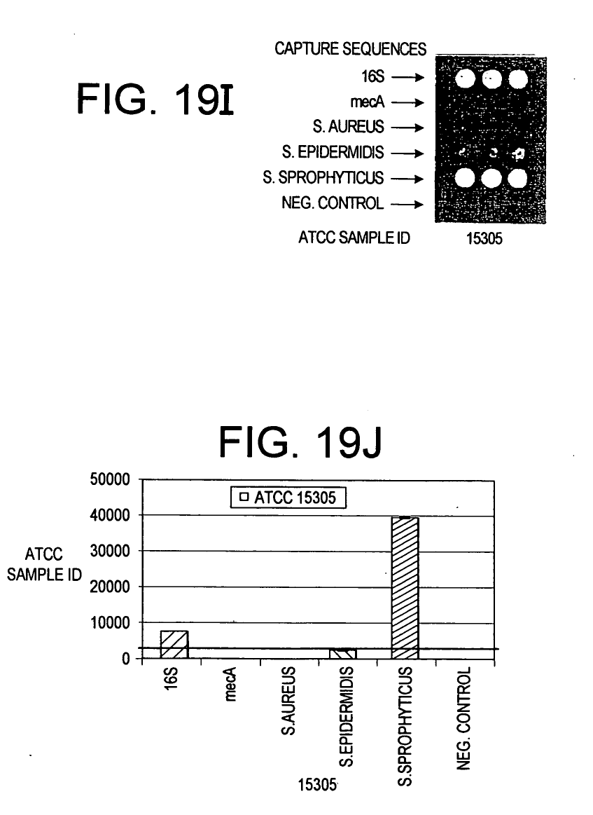

The results are shown in FIGS. 19(a) and (b). The species and methicillin resistance status of five selected Staphylococcus samples (see Table 3 below) were correctly identified using PCR amplicons demonstrating the specificity of the array sequences when standard PCR amplification procedures are employed.

TABLE 3

| ATCC Sample | |
|---|---|
| ID # | Description |
| 35556 | S. aureus |
| 700699 | S. aureus, Mu50-resistant to methicillin |
| 12228 | S. epidermidis |
| 35984 | S. epidermidis, RP62A-multiply antibiotic-resistant |
| 15305 | S. saprophyticus |

### Example 7

Staphylococcal Speciation and Methicillin Resistance Assay using Genomic DNA and Gold Nanoparticle-Labeled mecA, 16S and Tuf Probes

In this Example, the identification of Staphylococcus genus, species, and antibiotic resistance status was tested using total genomic DNA isolated from S. aureus and S. epidermidis bacterial cells. The genomic DNA samples tested were characterized by ATCC as described in table 3 above. The microarray plates and detection probes used for testing in example 6 also were used for this example. The microarray plates and capture and detection probes were prepared as described in Example 3. The genomic DNA samples were prepared as described in example 5. Each reaction consisted of 50 ul of 5×SSC, 0.05% Tween 20, 0.01% BSA, 200 pM each nanoparticle probe, and 15% formamide and 3.3 ng/ul of sonicated genomic DNA. The reagents were hybridized for 2 hrs at 40 C and 90% humidity. The microarray surface was washed for 30 sec in 0.5 M NaNO₃ at room temperature. The microarray was dried and exposed with silver development using commercial grade silver enhancer solutions (Silver Enhancer Kit, Catalog No. SE-100, Sigma, St. Louis) for 4 minutes. The silver stained microarray plate was then washed, dried and imaged using an Arrayworx® scanner (Model No. AWE, Applied Precision, Issaquah, Wash.).

The results are shown in FIG. 20. Significantly, Staphylococcus species and antibiotic resistance status was correctly identified for three genomic DNA samples tested based on net signal intensities that were above 3 standard deviations over background at only the correct capture probe site in each sample. This experiment demonstrates that even single nucleotide mutations can be detected within the tuf gene when Staphylococcus genomic DNA is hybridized to the array and labeled with silver amplified gold nanoparticle probes. Therefore, speciation of biological microorganisms that differ by as little as a single nucleotide within a given gene sequence is achievable by this novel detection methodology without any enzyme-based target amplification (e.g. PCR) or signal amplification (e.g. horseradish peroxidase) procedures.

The assay sensitivity was measured by titrating known amounts of total genomic DNA isolated from methicillin resistant S. aureus cells into the assay and measuring the net signal intensity from the mecA gene capture probes, FIG. 21. The lowest detectable quantity was 34 ng, which corresponds to roughly 10 million copies of the genome. Further optimization of the described detection procedures should enable much lower quantities of genomic DNA to be detectable.

### Example 8

General Protocol for DNA Isolation from Buccal Swabs.

A buccal swab sample is collected by rolling a sterile CytoSoft cytology brush (or equivalent) on the inside of each cheek 10-20 times. The collected sample was released into ˜500 μL cell lysis buffer by using a twirling motion. Typically, the lysis buffer comprised proteases (for example, Protease mix (Qiagen) at a final concentration of 2-10 mg/mL or Proteinase K at 1-5 mg/mL). The sample was incubated for 10 minutes at 65° C. at which point a fragmentation buffer was added to the solution and the sample was incubated for an additional 5 minutes at 65° C. The fragmentation buffer contained mild oxidants such as perborate or percarbonate and was formulated such that when added to the sample the concentration of the oxidants is ˜0.5% w/v. In order to stabilize the oxidant, the pH is maintained between 8-9 by using borate buffers in the presence of polymers such as polyvinylalcohol (0.01% w/v). The fragmentation buffer effected DNA fragmentation, an important requirement for the PCR-less SNP detection assay. Next, the DNA was selectively isolated by using CTAB in conjunction with magnetic microbeads, typically containing a silica surface. Magnetic beads (20-100 μg) were added to the solution and the CTAB and NaCl concentrations were raised to ˜1% and 0.3 M, respectively. The DNA was allowed to condense over the course of a short (˜10 min) incubation at 40° C. This was followed by an isolation of the magnetic beads with a magnetic separator. While the condensation does not require any alcohol, the DNA condensed on to the beads remained bound when washed with 80% ethanol. Repeated washing with 80% ethanol removed excess CTAB (or the condensing agents) and the DNA was released into either water or a hybridization buffer (50 μL). The isolated DNA was ready to be tested in PCR-less SNP assays by using published protocols (see Bao et al. in Nucleic Acids Res. (2005) 33(2):e15, which is incorporated by reference in its entirety).

### Example 9

Isolation of DNA from Buccal Samples.

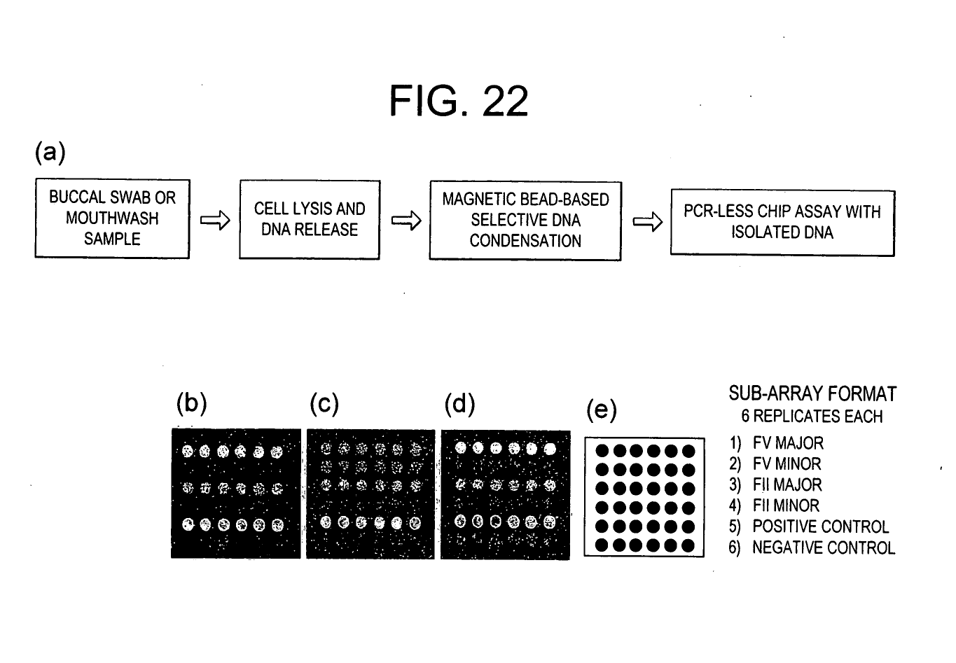

Sample collection was by using a Cytosoft brush (MPC) supplied to donors who were instructed to roll the brush on the inside of each cheek 20 times. The swab was then placed in a tube containing 600 μL cell lysis-binding buffer and heated for 10 minutes at 65° C. followed by a 2 minutes heating at 95° C. To the lysed sample were added NaCl and CTAB to a final concentration of 0.3 M and 1% CTAB and the solution was incubated at 40° C. to permit CTAB-mediated DNA condensation. Magnetic beads were added either along with the CTAB or after the incubation at 40° C., either way gives identical results. The beads which captured the DNA were isolated using a magnetic separator and washed with 80% ethanol twice to remove interferents including polysaccharides and lipids. The DNA was eluted into water or into a hybridization buffer and employed in PCR-less assays. FIG. 22 shows the results from a PCR-less assay showing good SNP discrimination in two genes with controls and buccal swab samples.

### Example 10

β Isolation of DNA from Mouthwash Samples.

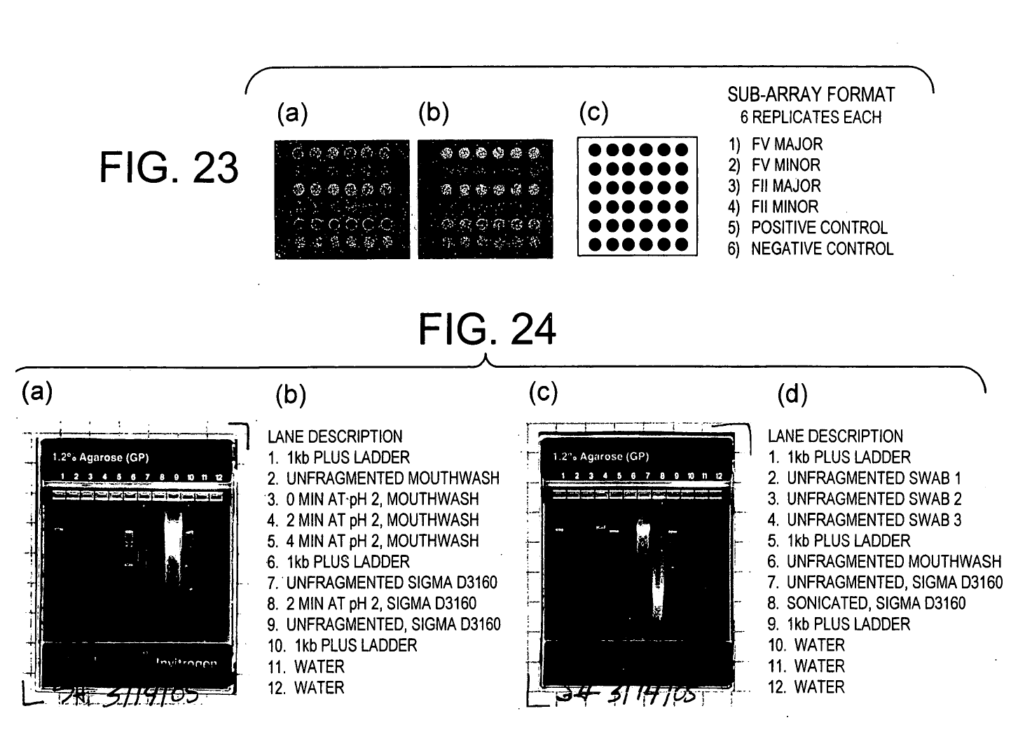

Mouthwash samples were obtained by swishing 10 mL water for 60 seconds. In the experiment, 1 mL of the sample was removed and subjected to the cell lysis as described in Example 8 and subsequently tested in PCR-less assays. FIG. 23 shows the results from the assay showing that there is sufficient amount of DNA for successful genotyping.

### Example 11

DNA Size Distribution Isolated using the Lysis-Binding Buffer.

The DNA samples obtained in Examples 8 and 9 were run on an agarose gel to determine the status of the DNA. Samples tested were both from a buccal swab and from mouthwash samples. While the distribution ranges were quite large, a significant amount of DNA had lengths below 1 kb, a length that is ideal for the PCR-less SNP detection. FIG. 24 shows the results of the DNA size distribution analysis.

### Example 12

β Isolation of DNA from Other Samples.

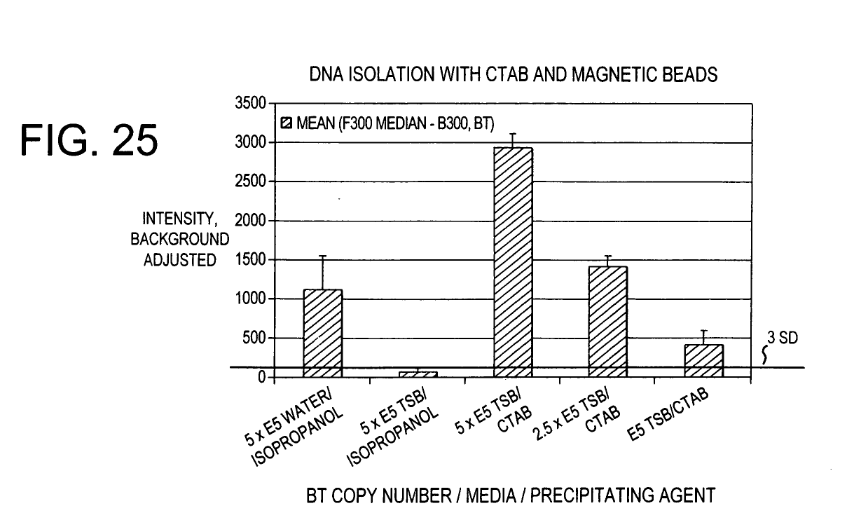

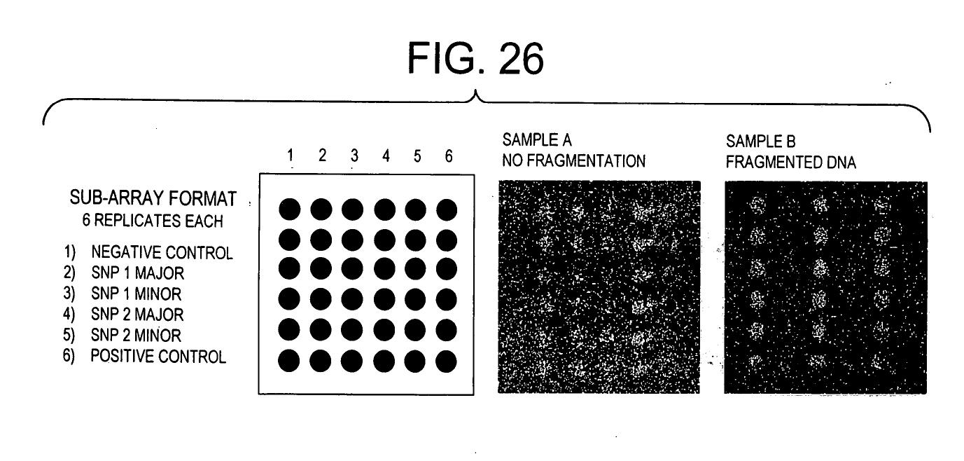

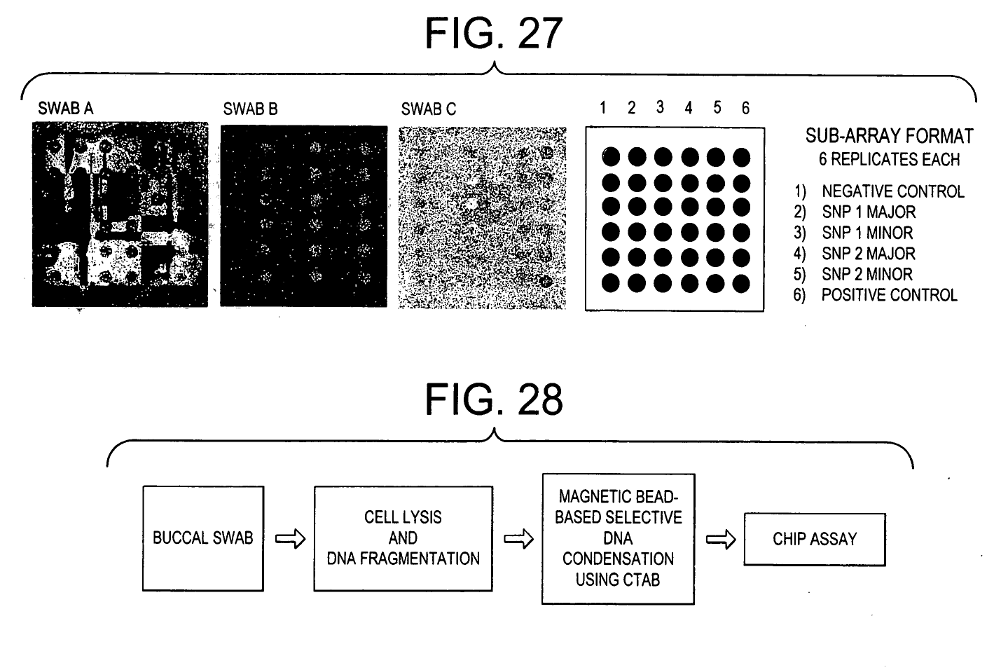

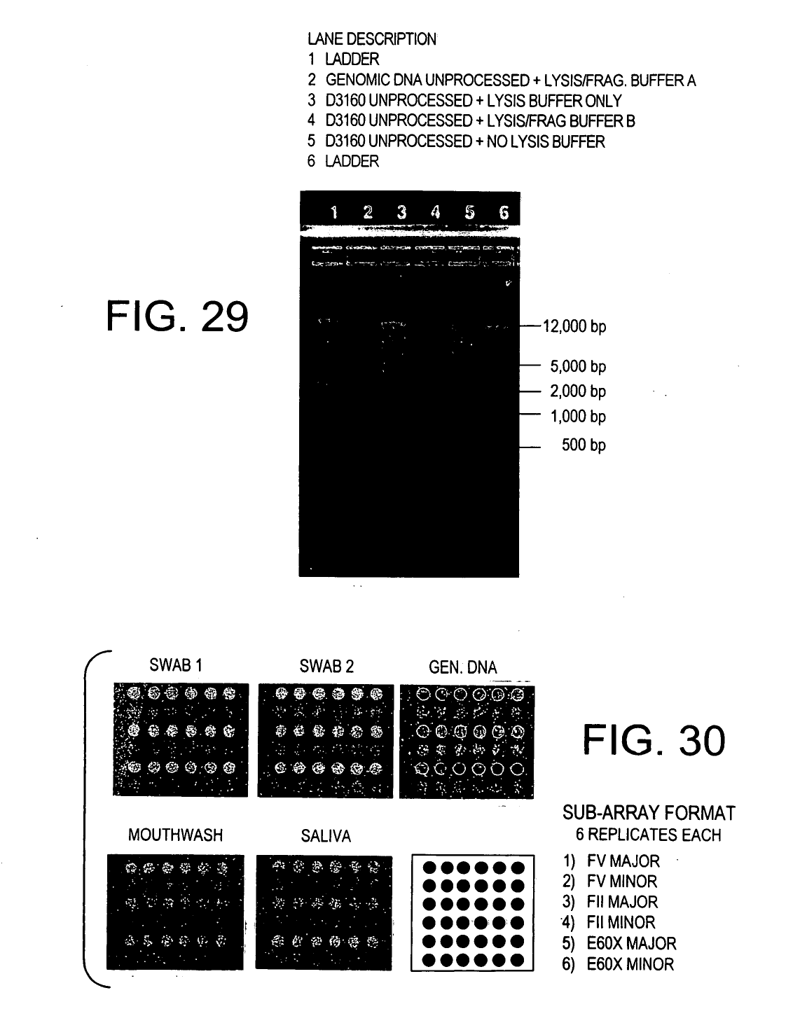

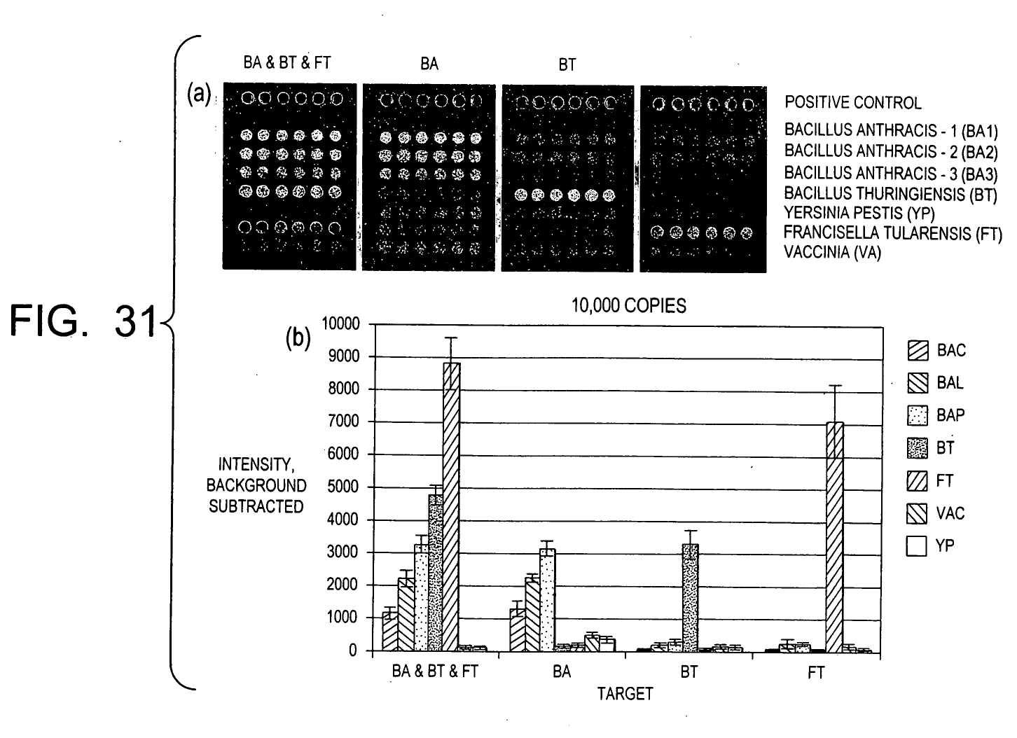

DNA was isolated from samples containing small amounts of DNA, typically less than 1 ng genomic DNA. To spent media samples derived from bacterial cultures with concentrations lower than 10⁶ cfu/mL an equal volume was added to the lysis buffer. The remaining steps were similar to those described for Example 8. Total genomic DNA isolated from bacterial spent media was employed in microarray detection assays designed to detect organism-specific genes. FIG. 25 shows the specific detection of three genes specific to B. anthracis, genomic DNA for which was isolated from spent media using the above isolation protocol. FIG. 31 shows the ability of this procedure to isolate and simultaneously detect 10,000 copies of various bacterial targets (B. anthracis, B. thuringiensis, and F. tularensis) from 800 μL of sample volume. The starting concentration of the target is an unprecedented 10 attomolar or 10×10⁻¹⁸ M.

It should be understood that the foregoing disclosure emphasizes certain specific embodiments of the invention and that all modifications or alternatives equivalent thereto are within the spirit and scope of the invention as set forth in the appended claims.

What we claim is:

1. A method for detecting one or more target nucleic acid sequences in a sample, the sample comprising nucleic acid molecules of higher biological complexity relative to amplified nucleic acid molecules and the one or more target nucleic acid sequences each differ from known nucleic acid sequences by at least one nucleotide, the method comprising the steps of:
a) admixing a sample to a lysis buffer, wherein the lysis buffer comprises at least one detergent;
b) fragmenting the nucleic acids molecules of step (a);
c) condensing the fragmented nucleic acid molecules onto a binding substrate in the presence of CTAB and NaCl so as to bind the nucleic acid molecules onto a surface of the substrate;
d) washing the binding substrate having the bound nucleic acid molecules;
e) eluting the bound nucleic acid molecules from the binding substrate;
f) providing an addressable substrate having a plurality of capture oligonucleotides bound thereto, wherein the capture oligonucleotides have sequences that are complementary to one or more portions of the one or more target nucleic acid sequences;
g) providing one or more detector probes comprising detector oligonucleotides, wherein the detector oligonucleotides have sequences that are complementary to one or more portions of the one or more target nucleic acid sequences of step (f) that are not recognized by a capture oligonucleotide on the substrate;
h) contacting the nucleic acid molecules of step (e) with the substrate and the detector probes under conditions that are effective for the hybridization of the capture oligonucleotides to one or more portions of the one or more target nucleic acid sequences and the hybridization of the detector probes to portions of the one or more target nucleic acid sequences that are not recognized by a capture oligonucleotide and to allow for discrimination between targets that differ by at least one nucleotide; and
i) detecting whether any of the capture oligonucleotide and detector probes hybridized with any of the target nucleic acid sequences.

2. The method of claim 1 wherein the lysis buffer further comprises at least one protease and at least one salt.

3. The method of claim 1, wherein the fragmentation is carried out in the presence of at least one oxidant, DNases, restriction enzymes, an acid or by ultrasonication.

4. The method of claim 1, wherein subsequent to step (b) but prior to step (c) further comprises adding an aqueous solution comprising CTAB and NaCl.

5. The method of claim 1, wherein step (a) admixing the sample to the lysis buffer and step (b) fragmenting the nucleic acid molecules are carried out in a single step, and wherein the lysis buffer further comprises at least one protease, at least one salt, and at least one oxidant.

6. The method of claim 1, wherein step (a) admixing the sample to a lysis buffer, step (b) fragmenting and step (c) condensing the nucleic acid molecules are carried out in a single step, and wherein the lysis buffer further comprises at least one protease, at least one salt, at least one oxidant and CTAB.

7. The method of claim 1, wherein the lysis buffer further comprises at least one salt and at least one polymeric compound.

8. The method of claim 7, wherein the polymeric compound is selected from the group consisting of polyvinyl alcohol and polyethylene glycol.

9. The method of claim 1, wherein the lysis buffer further comprises at least one salt, at least one polymeric compound, at least one protease, and at least one lipase.

10. The method of claim 1, wherein the lysis buffer further comprises at least one salt, at least one polymeric compound, at least one protease, and at least one mucolytic compound.

11. The method of 10, wherein the mucolytic compound is selected from the group consisting of N-Acetyl-L-cysteine and lysozyme.

12. The method of claim 1, wherein the step (a) admixing the sample to the lysis buffer and step (b) fragmenting the nucleic acid molecules are carried out in a single step, wherein the lysis buffer further comprises at least one salt, and at least one oxidant.

13. The method of claim 2, wherein the protease in the lysis buffer is selected from the group consisting of endoproteases and exoproteases.

14. The method of claim 13, wherein the exoproteases are selected from the group consisting of Proteinase K, Bromelain, papain and ficin.

15. The method of claim 3, wherein the oxidant is selected from the group consisting of perborate, percarbonate, hydrogen peroxide and peroxymonosulfate.

16. The method of claim 1, wherein the binding substrate is magnetic microbeads containing a silica surface.

17. The method of claim 1, wherein washing of the binding substrate having the bound nucleic acid molecules comprises washing with 80% ethanol to remove excess CTAB.

18. The method of claim 1, wherein the target nucleic acid sequence comprises a Single Nucleotide Polymorphism.

19. The method of claim 1, wherein the single nucleotide difference is recognized by the capture oligonucleotide bound to the substrate.

20. The method of claim 1, wherein the single nucleotide difference is recognized by the detector oligonucleotides.

21. The method of claim 1, wherein the target nucleic acid molecules comprise genomic DNA, genomic RNA, expressed RNA, plasmid DNA, mitochondrial or other cell organelle DNA, free cellular DNA, viral DNA or viral RNA, or a mixture of two or more of the above.

22. The method of claim 1, wherein the substrate comprises a plurality of capture oligonucleotides, each of which can recognize a different single nucleotide polymorphism.

23. The method of claim 1, wherein the sample comprises more than one nucleic acid target, each of which comprises a different single nucleotide polymorphism.

24. The method of claim 1, wherein one or more types of detector probes are provided, each of which has detector oligonucleotides bound thereto that are capable of hybridizing with a different nucleic acid target.

25. The method of claim 1, wherein sample is contacted with the detector probe so that a nucleic acid target present in the sample hybridizes with the detector oligonucleotides on the detector probe, and the nucleic acid target bound to the detector probe is then contacted with the substrate so that the nucleic acid target hybridizes with the capture oligonucleotide on the substrate.

26. The method of claim 1, wherein sample is contacted with the substrate so that a nucleic acid target present in the sample hybridizes with a capture oligonucleotide, and the nucleic acid target bound to the capture oligonucleotide is then contacted with the detector probe so that the nucleic acid target hybridizes with the detector oligonuclotides on the detector probe.

27. The method of claim 1, wherein the sample is contacted simultaneously with the detector probe and the substrate.

28. The method of claim 1, wherein the detector probe comprise a detectable label.

29. The method of claim 28, wherein the detection label allows detection by photonic, electronic, acoustic, opto-acoustic, gravity, electrochemical, electro-optic, mass-spectrometric, enzymatic, chemical, biochemical, or physical means.

30. The method of claim 28, wherein the label is fluorescent, luminescent, phosphorescent, radioactive, a nanoparticle, a dendrimer, a molecular aggregate, a quantum dot, or a bead.

31. The method of claim 1, wherein the detector probe is a nanoparticle probe having detector oligonucleotides bound thereto.

32. The method of claim 31, wherein the nanoparticles are made of a noble metal.

33. The method of claim 32, wherein the nanoparticles are made of gold or silver.

34. The method of claim 33, wherein the nanoparticles are made of gold.

35. The method of claim 31, wherein the detecting comprises contacting the substrate with silver stain.

36. The method of claim 31, wherein the detecting comprises observation of light scattered by the nanoparticle.

37. The method of claim 31, wherein the detecting comprises observation with an optical scanner.

38. The method of claim 30, wherein the detecting comprises observation with a flatbed scanner.

39. The method of claim 37 or 38, wherein the scanner is linked to a computer loaded with software capable of calculating grayscale measurements, and the grayscale measurements are calculated to provide a quantitative measure of the amount of nucleic acid detected.

40. The method of claim 31, wherein the oligonucleotides attached to the substrate are located between two electrodes, the nanoparticles are made of a material that is a conductor of electricity, and step (i) comprises detecting a change in conductivity.

41. The method of claim 40, wherein the electrodes are made of gold and the nanoparticles are made of gold.

42. The method of claim 40, wherein the substrate is contacted with silver stain to produce the change in conductivity.

43. The method of claims 31, wherein a plurality of oligonucleotides, each of which can recognize a different target nucleic acid sequence, are attached to the substrate in an array of spots and each spot of oligonucleotides is located between two electrodes, the nanoparticles are made of a material that is a conductor of electricity, and step (i) comprises detecting a change in conductivity.

44. The method of claim 43, wherein the electrodes are made of gold and the nanoparticles are made of gold.

45. The method of claim 43, wherein the substrate is contacted with silver stain to produce the change in conductivity.

46. A method for identifying one or more single nucleotide polymorphisms in a sample, the sample comprising nucleic acid molecules of higher biological complexity relative to amplified nucleic acid molecules, the method comprising the steps of:
a) admixing a sample to a lysis buffer, wherein the lysis buffer comprise at least one detergent;
b) fragmenting the nucleic acids molecules of step (a);
c) condensing the fragmented nucleic acid molecules onto a binding substrate in the presence of CTAB and NaCl so as to bind the nucleic acid molecules onto a surface of the substrate;
d) washing the binding substrate having the bound nucleic acid molecules;
e) eluting the bound nucleic acid molecules from the binding substrate;
f) providing an addressable substrate having a plurality of capture oligonucleotides bound thereto, wherein the capture oligonucleotides have sequences that are complementary to multiple portions of a nucleic acid target, each said portion comprising a specific polymorphism;
g) providing one or more detector probes comprising detector oligonucleotides, wherein the detector oligonucleotides have a sequence that is complementary to at least a portion of one of the nucleic acid targets of step (f) that is not recognized by a capture oligonucleotide on the substrate;
h) contacting the nucleic acid molecules of step (e) with the substrate and the detector probes under conditions that are effective for the hybridization of the capture oligonucleotides to multiple portions of the nucleic acid target and the hybridization of the detector probe to the nucleic acid target and to allow for discrimination between targets that differ by a single nucleotide; and
i) detecting whether any of the capture oligonucleotides and detector probes hybridized with any of the nucleic acid targets.

47. The method of claim 46, wherein the lysis buffer further comprises at least one protease and at least one salt.

48. The method of claim 46, wherein the fragmentation is carried out in the presence of at least one oxidant, DNases, restriction enzymes, an acid or by ultrasonication.

49. The method of claim 46, wherein subsequent to step (b) but prior to step (c) further comprises adding an aqueous solution comprising CTAB and NaCl.

50. The method of claim 46, wherein step (a) admixing the sample to the lysis buffer and step (b) fragmenting the nucleic acid molecules are carried out in a single step, and wherein the lysis buffer further comprises at least one protease, at least one salt, and at least one oxidant.

51. The method of claim 46, wherein step (a) admixing the sample to a lysis buffer, step (b) fragmenting and step (c) condensing the nucleic acid molecules are carried out in a single step, and wherein the lysis buffer further comprises at least one protease, at least one salt, at least one oxidant and CTAB.

52. The method of claim 46, wherein the lysis buffer further comprises at least one salt and at least one polymeric compound.

53. The method of claim 52, wherein the polymeric compound is selected from the group consisting of polyvinyl alcohol and polyethylene glycol.

54. The method of claim 46, wherein the lysis buffer further comprises at least one salt, at least one polymeric compound, at least one protease, and at least one lipase.

55. The method of claim 46, wherein the lysis buffer further comprises at least one salt, at least one polymeric compound, at least one protease, and at least one mucolytic compound.

56. The method of 55, wherein the mucolytic compound is selected from the group consisting of N-Acetyl-L-cysteine and lysozyme.

57. The method of claim 46, wherein the step (a) admixing the sample to the lysis buffer and step (b) fragmenting the nucleic acid molecules are carried out in a single step, wherein the lysis buffer further comprises at least one salt, and at least one oxidant.

58. The method of claim 47, wherein the protease in the lysis buffer is selected from the group consisting of endoproteases and exoproteases.

59. The method of claim 58, wherein the exoproteases are selected from the group consisting of Proteinase K, Bromelain, papain, and ficin.

60. The method of claim 58, wherein the oxidant is selected from the group consisting of perborate, percarbonate, hydrogen peroxide and peroxymonosulfate.

61. The method of claim 56, wherein the binding substrate is magnetic microbeads containing a silica surface.

62. The method of claim 46, wherein washing of the binding substrate having the bound nucleic acid molecules comprises washing with 80% ethanol to remove excess CTAB.

63. The method of claim 56, wherein the polymorphism is recognized by the capture oligonucleotide bound to the substrate.

64. The method of claim 46, wherein the polymorphism is recognized by the detector oligonucleotides.

65. The method of claim 46, wherein the nucleic acid molecules in the sample comprise genomic DNA, genomic RNA, expressed RNA, plasmid DNA, mitochondrial or other cell organelle DNA, free cellular DNA, viral DNA or viral RNA, or a mixture of two or more of the above.

66. The method of claim 46, wherein the substrate comprises a plurality of capture oligonucleotides, each of which can recognize one or more different single nucleotide polymorphisms.

67. The method of claim 46, wherein the sample comprises more than one nucleic acid targets, each of which comprises a different single nucleotide polymorphism.

68. The method of claim 46, wherein one or more types of detector probes are provided, each of which has detector oligonucleotides bound thereto that are capable of hybridizing with a different nucleic acid target.

69. The method of claim 46, wherein sample is contacted with the detector probe so that a nucleic acid target present in the sample hybridizes with the detector oligonucleotides on the detector probe, and the nucleic acid target bound to the detector probe is then contacted with the substrate so that the nucleic acid target hybridizes with the capture oligonucleotide on the substrate.

70. The method of claim 46, wherein sample is contacted with the substrate so that a nucleic acid target present in the sample hybridizes with a capture oligonucleotide, and the nucleic acid target bound to the capture oligonucleotide is then contacted with the detector probe so that the nucleic acid target hybridizes with the detector oligonuclotides on the detector probe.

71. The method of claim 46, wherein the sample is contacted simultaneously with the detector probe and the substrate.

72. The method of claim 46, wherein the detector oligonucleotides comprise a detectable label.

73. The method of claim 72, wherein the detection label allows detection by photonic, electronic, acoustic, opto-acoustic, gravity, electro-chemical, electro-optic, mass-spectrometric, enzymatic, chemical, biochemical, or physical means.

74. The method of claim 72, wherein the label is fluorescent, luminescent, phosphorescent, radioactive, a nanoparticle, a dendrimer, a molecular aggregate, a quantum dot, or a bead.

75. The method of claim 46, wherein the detector probe is a nanoparticle probe having detector oligonucleotides bound thereto.

76. The method of claim 75, wherein the nanoparticles are made of a noble metal.

77. The method of claim 76, wherein the nanoparticles are made of gold or silver.

78. The method of claim 77, wherein the nanoparticles are made of gold.

79. The method of claim 75, wherein the detecting comprises contacting the substrate with silver stain, detecting light scattered by the nanoparticle, observation with an optical scanner, or observation with a flatbed scanner.

80. The method of claim 79, wherein the scanner is linked to a computer loaded with software capable of calculating grayscale measurements, and the grayscale measurements are calculated to provide a quantitative measure of the amount of nucleic acid detected.

81. The method of claim 75, wherein the oligonucleotides attached to the substrate are located between two electrodes, the nanoparticles are made of a material that is a conductor of electricity, and step (i) comprises detecting a change in conductivity.

82. The method of claim 81, wherein the electrodes are made of gold and the nanoparticles are made of gold.

83. The method of claim 81, wherein the substrate is contacted with silver stain to produce the change in conductivity.

84. The method of claims 81, wherein a plurality of oligonucleotides, each of which can recognize a different single nucleotide polymorphism, are attached to the substrate in an array of spots and each spot of oligonucleotides is located between two electrodes, the nanoparticles are made of a material that is a conductor of electricity, and step (i) comprises detecting a change in conductivity.

85. The method of claim 84, wherein the electrodes are made of gold and the nanoparticles are made of gold.

86. The method of claim 84, wherein the substrate is contacted with silver stain to produce the change in conductivity.

87. The method of claim 1 or claim 46, wherein the higher biological complexity is greater than about 50,000.

88. The method of claim 1 or claim 46, wherein the higher biological complexity is between about 50,000 and about 50,000,000,000.

89. The method of claim 1 or claim 46, wherein the higher biological complexity is about 1,000,000,000.

90. The method of claim 1, wherein the target nucleic acid sequence is a portion of a gene of a biological organism.

91. The method of claim 1, wherein the target nucleic acid sequence is a portion of a gene of a Staphylococcus bacterium.

92. The method of claim 91, wherein the Staphylococcus bacterium is S. aureus, S. haemolyticus, S. epidermidis, S. lugdunensis, S. hominis, or S. saprophyticus.

93. The method of claim 91, wherein the target nucleic acid sequence is a portion of the Tuf gene, a portion of the femA gene, a portion of the 16S rRNA gene, a portion of the hsp60 gene, a portion of the sodA gene, or a portion of the mecA gene.

94. The method of claim 1, wherein the target nucleic acid sequence comprises the sequence set forth in SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 24, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, or SEQ ID NO: 78.

95. The method of claim 1, wherein at least one of the detection oligonucleotides comprise the sequence set forth in SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 24, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, or SEQ ID NO: 78.

96. The method of claim 1, wherein the capture oligonucleotide comprises the sequence set forth in SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 24, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, or SEQ ID NO: 78.

97. The method of claim 1, wherein at least one of the capture oligonucleotides comprise the sequence set forth in SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 24, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, or SEQ ID NO: 78.

99. The method of claim 1, wherein at least one of the target nucleic acid sequences is a portion of a gene of a Staphylococcus bacterium and at least one of the target nucleic acid sequences is a portion of the mecA gene.

100. The method of claim 1, wherein the method is used to distinguish between two or more species of a common genus.

101. The method of claim 100, wherein the species differ by two or more non-consecutive nucleotides, by two or more consecutive nucleotides, or by at least one nucleotide.
